(19) United States
(12) Patent Application Publication
(10) Pub. No.: US 2008/0249546 A1
(43) Pub. Date: Oct. 9, 2008

(54) ANASTOMOSIS SYSTEMS AND METHODS

(76) Inventors: Jeffrey D. Sandstrom, Forest Lake, MN (US); Eric A. Meyer, Andover, MN (US); Damian A. Jelich, Cottage Grove, MN (US); Robert H. Reetz, Rockford, MN (US); Roderick E. Briscoe, Rogers, MN (US)

Correspondence Address: Medtronic CardioVascular, 7000 Central Avenue, Minneapolis, MN, 54432, US

(21) Appl. No.: 11/970,319

(22) Filed: Jan. 7, 2008

Related U.S. Application Data
(60) Provisional application No. 60/883,615, filed on Jan. 5, 2007.

(51) Int. Cl. A61B 17/08

(52) U.S. Cl. 606/153

(57) ABSTRACT

A surgical connection apparatus including a plurality of delivery assemblies, a housing assembly, and a drive mechanism. The delivery assemblies each include an outer member, an inner member, and optionally a retention member. The inner member is disposed within the outer member, terminates at a distal tip and forms an internal passage that is open at a window. The delivery assemblies releasably retain a self-closing clip. The housing assembly maintains the delivery assemblies in a generally circular arrangement. The drive mechanism operates to transition the delivery assembly in releasing a self-closing clip from the corresponding delivery assembly. With this construction, a plurality of self-closing clips can be simultaneously deployed in a manner effectuating, for example, an end-to-side anastomosis.

### CROSS-REFERENCE TO RELATED APPLICATIONS

This application claims priority under 35 U.S.C. §119(e)(1) to U.S. Provisional Patent Application Ser. No. 60/883,615, filed Jan. 5, 2007, entitled “Anastomosis Systems and Methods”, and bearing Attorney Docket No. M190.200.101/P0023238.00; and the entire teachings of which are incorporated herein by reference.

### BACKGROUND

The present disclosure relates to systems and methods for surgically joining structures. More particularly, the present disclosure can involve anastomosing a tubular graft structure to a second structure, for example in a proximal anastomosis.

Arterial replacement or bypass grafting has been performed for many years using open surgical techniques and a variety of prosthetic grafts. These grafts can be manufactured as fabrics, or prepared as autografts (from the patient's own tissues) or heterografts (from the tissues of animals or a combination of tissues), semi-synthetic tissues and/or alloplastic materials. A graft can be joined to the involved artery in a number of different positions, including end-to-end, end-to-side, and side-to-side. This attachment between artery and graft is known as an anastomosis. Constructing an arterial anastomosis is technically challenging for a surgeon in open surgical procedures, and is almost a technical impossibility using minimally invasive techniques.

For example, coronary artery bypass graft (CABG) surgery is a surgical procedure performed in severe cases of coronary blockages. CABG procedures entail anastomosing an artery to a vascular graft that restores the flow of blood by establishing another pathway around the occluded vasculature. During CABG surgery, a vein or other conduit can be attached proximally to the patient's aorta. The other end is attached to the blocked artery, downstream from the obstruction, thus bypassing the coronary occlusion. CABG procedures can be done by placing the patient on a heart-lung machine and stopping the heart from beating, or can be done on a beating heart without a heart-lung machine. One problem encountered in either CABG procedure approach is the need to perform the procedure while simultaneously maintaining sufficient function of the patient's circulatory system.

In the case where a CABG procedure involves arresting the heart so that blood flow is diverted from the vessel to be anastomosed, the patient's blood circulation is maintained by a cardiopulmonary bypass (CPB). This bypass is accomplished by diverting the blood flow at selected arterial locations. The blood is diverted to the bypass system for release of carbon dioxide and subsequent oxygenation. Then, the blood is returned to the patient via a pump. Examples of these procedures are found in U.S. Pat. Nos. 5,799,661 and 5,452,733.

Although the beating heart CABG procedure eliminates the need for CPB, it has required diverting blood flow from a proximal anastomosis, such as one that attaches graft material (e.g., a graft vessel) to the ascending aorta. To attach the graft to the aorta in a beating heart situation, surgeons have typically used a “side-biting clamp” that isolates the aortic region where the anastomosis will be performed. This allows the surgeon to create the anastomosis without the site being exposed to the high-pressure blood flow of the normal aorta.

Among the drawbacks associated with aortic clamping is an increased chance of trauma to the arteries caused by ligatures at the clamped site, the possible dislodging of plaque within the clamped vessel wall. As mentioned above, the arterial bypass may be required due to the deposits of plaque that have occluded the vessel. However, the plaque is typically present throughout the artery, and is not limited to the occluded location. Clamping the artery creates a risk of plaque being released into the blood stream that in turn has the potential of causing a stroke, occlusion of a smaller peripheral vessel, or other vascular trauma. In a beating heart procedure, full clamping (i.e., cross-clamping) of the aorta for graft attachment at the proximal anastomosis is not feasible. Therefore, a side-biting clamp is used to clamp off only a portion of the cross-section of the aorta, where the proximal anastomosis is performed. This type of clamping procedure poses the same risks described above with respect to cross-clamping (e.g., the risk of release of plaque and resultant cause of a stroke, occlusion of a smaller peripheral vessel or other vascular trauma).

Other attempts to address problems relating to blood flow diversion include diverting the blood by placing a balloon catheter within the aorta, for example as described in U.S. Pat. No. 5,868,702. Potential drawbacks with this approach include the possibility of distributing plaque deposits and creating particles in the bloodstream, the chance that the balloon catheter may move within the aorta thereby disrupting the seal and resulting in blood loss, and/or trauma to aortic tissue caused by the pressure needed to create the seal.

In addition to the circulatory concerns above, many factors contribute to the difficulty of performing an arterial replacement or bypass grafting. One conventional anastomosis technique entails the surgeon hand suturing the graft to the involved artery. In general terms, a suture has a suture needle that is attached or “swaged on” to a long, trailing suture material. The needle must be precisely controlled and accurately placed through both the graft and artery in question. The trailing suture material must be held with proper tension to keep the graft and artery together, and must be carefully manipulated to prevent the suture material from tangling. In open surgery, these maneuvers can usually be accomplished within a necessary timeframe, thus avoiding subsequent tissue damage (or tissue death) that can result from prolonged occlusion of arterial blood flow. As a point of reference, the surgeon must complete the graft in as little time possible due to the absence of blood flowing through the artery. If blood flow is not promptly restored, sometimes in as little as thirty minutes, the tissues the artery supplies may experience significant damage or necrosis.

Additional factors contribute to the difficulty of performing an anastomosis. For example, the tissues to be joined must be precisely aligned with respect to each other to ensure the integrity and patency of the anastomosis. If one of the tissues is affixed too close to its edge, the suture can rip through the tissue and impair both the tissue and the anastomosis. Another factor is that, even after the tissues are properly aligned, it is difficult and time consuming to pass the needle through the tissues, form the knot in the suture material, and ensure that the suture material does not become tangled. These difficulties are exacerbated by the small size of the artery and graft. The arteries subject to peripheral, vascular, and cardiovascular surgery typically range in diameter from several millimeters to several centimeters. A graft is typically about the same size as the artery to which it is being attached.

In light of the above, efforts have been made to improve and simplify surgical connection procedures, such as anastomosis procedures, as part of CABG surgery. For example, U.S. Pat. No. 6,358,258 describes systems and methods for performing anastomosis or attachments of bodily ducts that are asserted to simplify suture delivery in both stopped heart and beating heart procedures, and to be suitable for use in a minimally invasive environment using percutaneous ports, or with retractor systems, or in a generally open surgery environment. U.S. Pat. No. 6,682,540 describes systems and methods for placing multiple sutures during anastomosis, including a crown having a plurality of strands connected together by one or more circular bands. U.S. Pat. No. 6,461,365 describes surgical clips and methods of tissue approximation and attachment that are asserted as being useful in open surgical procedures as well as endoscopic and other minimally-invasive procedures. In this regard, one known system for applying a clip around tissues to be joined in anastomosis is disclosed in a brochure entitled “VCS Clip Applier System,” published in 1995 by Auto Suture Company, a division of U.S. Surgical Corporation. A clip is applied by situating an instrument about the tissue in a non-penetrating manner (i.e., the clip does not penetrate through the tissues, but rather is clamped down around the tissues). As previously explained, it is imperative in forming an anastomosis that tissues to be joined are properly aligned with respect to each other. The disclosed VCS Clip Applier has no means for positioning tissues. Before the clip can be applied, the tissues must first be properly positioned with respect to each other, for example by skewering the tissues with a needle as discussed above in common suturing techniques, or with forceps to bring the tissues together. It is extremely difficult to perform such positioning techniques in minimally invasive procedures.

Not withstanding the above, use of self-closing clips as a replacement for sutures in performing an anastomosis procedure is highly desired by surgeons. In this regard, the design and construction of self-closing clips or fasteners has greatly improved as described, for example in U.S. Pat. No. 6,945,980 (the teachings of which are incorporated herein by reference). Along these same lines, efforts have been made to develop surgical tools or instruments that can simultaneously deliver or deploy two or more of the self-closing clips in a simple, consistent fashion. One such instrument is available from Medtronic, Inc., of Minneapolis, Minn. under the trade name Spyder®. The Spyder® device provides fast, automated proximal anastomotic connection by simultaneous delivery of multiple U-Clip™ self-closing surgical clips (or other self-closing clips) without the use of a side-biting clamp or second manipulation of the aorta. Other instruments for delivering self-closing clips are described, for example, in U.S. Publication Nos. 2004/0068276 and 2005/0070924, the teachings of both of which are incorporated herein by reference. In light of the enthusiasm with which these and similar systems have been received, any further improvement would be welcomed.

In light of the above, a need exists for improved systems and methods for performing surgical connections, such as anastomotic procedures.

### SUMMARY

Some aspects in accordance with principles of the present disclosure relate to a surgical connection apparatus including a plurality of delivery assemblies, a housing assembly, and a drive mechanism. The plurality of delivery assemblies each include an outer member and an inner member. The outer member defines a lumen within which the inner member is slidably disposed. In this regard, the inner member terminates at a distal tip and forms an internal passage, external access to which is provided via a longitudinal window. The window is formed in a distal segment of the inner member. An optional retention member is slidably disposed within the inner member in some embodiments. Regardless, the delivery assemblies are each adapted to releasably retain a self-closing clip at least partially within the internal passage in a retained state. The housing assembly maintains the plurality of delivery assemblies such that distal regions of the delivery assemblies collectively define a generally circular arrangement. Finally, the drive mechanism operates to transition the delivery assemblies from the retained state in releasing a self-closing clip from the corresponding delivery assembly. With this construction, a plurality of self-closing clips can be simultaneously deployed in a manner effectuating an end-to-side anastomosis. More particularly, each of the self-closing clips are fully captured within a respective one of the delivery assemblies prior to deployment, with subsequent release of the clip via the delivery assembly/window arrangement promoting clip deployment in a desired order. In some embodiments, the drive mechanism is adapted to effectuate simultaneous release of the clips in response to depression of a single actuator button in a direction perpendicular to a direction of movement of the inner members. In other embodiments, the surgical connection apparatus further includes an adjustment device adapted to adjust a radial spacing of the delivery assembly distal regions via rotation of a ring.

Other aspects in accordance with principles of the present disclosure relate to a method of performing an anastomosis. The method includes providing a surgical connection apparatus including a plurality of delivery assemblies each defining a distal region and releasably retaining a self-closing clip. The distal regions are passed through a wall of a tubular graft structure adjacent a distal end thereof. The distal regions of the delivery assemblies are introduced into a hole formed in a second structure that otherwise defines an exterior surface opposite an interior surface. The plurality of self-closing clips are simultaneously deployed from the delivery assemblies such that the clips pass through the tubular graft structure and the second structure to secure the tubular graft structure and the second structure together. In this regard, upon securement, an interface region is defined in the tubular graft structure from the location at which the clips pass through the tubular graft structure and extending to the distal end thereof. With this in mind, at least a majority of the interface region abuts the exterior surface of the second structure. With this approach, a compliant, end-to-side anastomosis can be achieved that is similar to the anastomotic relationship achieved with a conventional, hand-sutured approach, but done so on an expedited basis. Along these lines, virtually any take-off angle, in addition to a right angle take-off arrangement, of the tubular graft structure relative to the second structure can be achieved.

Yet other aspects in accordance with principles of the present disclosure relate to a kit for performing anastomosis. The kit includes a surgical connection apparatus and a loading tray. The surgical connection apparatus includes a plurality of delivery assemblies each releasably retaining a self-closing clip at a distal region thereof. The delivery assemblies are maintained by a housing, with the surgical connection apparatus further including a drive mechanism for effectuating simultaneous release of the clips from respective ones of the delivery assemblies. The loading tray is adapted to facilitate loading of a tubular graft structure on to the surgical connection apparatus, and defines a loading station and a tool pocket. The loading station is adapted to maintain a tubular graft structure, whereas the pocket is sized to slidably receive the surgical connection apparatus. In this regard, the pocket is arranged relative to the loading station such that upon positioning of a tubular graft structure in the loading station, the surgical connection apparatus can be slid along the pocket to effectuate loading of the tubular graft structure onto the distal regions of the delivery assemblies. In some embodiments, the kit further includes a guide piece configured to effectuate desired location of the loaded tubular graft structure relative to the delivery assemblies via sliding of the delivery assemblies within the guide piece.

### BRIEF DESCRIPTION OF THE DRAWINGS

FIG. 1A is an exploded view of a surgical connection apparatus in accordance with aspects of the present disclosure;

FIG. 1B is a side perspective view of the apparatus of FIG. 1A upon final assembly, with a portion of a housing removed;

FIG. 2 is an exploded side view of a delivery assembly portion of the apparatus of FIG. 1A in conjunction with a self-closing clip;

FIG. 3A is a perspective view of a distal portion of an outer member of the delivery assembly of FIG. 2;

FIG. 3B is a transverse cross-sectional view of the outer member of FIG. 3A along the lines 3B-3B;

FIG. 4A is a perspective view of a distal segment of an inner member of the delivery assembly of FIG. 2;

FIG. 4B is a transverse cross-sectional view of the inner member of FIG. 4A;

FIG. 4C is a longitudinal cross-sectional view of the inner member of FIG. 4A;

FIG. 5A is a side plan view of the delivery assembly of FIG. 2 upon final assembly;

FIGS. 5B and 5C are cross-sectional views illustrating transitioning of the delivery assembly of FIG. 2 from a clip retained state to a clip deployment state;

FIG. 6A is a simplified plan view of a distal segment of another inner member useful with the delivery assembly of FIG. 2;

FIG. 6B is a simplified plan view of a distal segment of another inner member useful with the delivery assembly of FIG. 2;

FIG. 7 is an enlarged plan view of a self-closing clip useful with the apparatus of FIG. 1A;

FIG. 8 is a perspective, cross-sectional view of the apparatus of FIG. 1A;

FIG. 9A is a front perspective view of a first holder body component of the apparatus of FIG. 8;

FIG. 9B is a side perspective view of the first holder body of FIG. 9A;

FIG. 10A is a first perspective view of a second holder body component of the apparatus of FIG. 8;

FIG. 10B is a rear perspective view of the second holder body of FIG. 10A;

FIG. 10C is a top view of the second holder body of FIG. 10A;

FIG. 11 is a side view of a latch component of the apparatus of FIG. 8;

FIG. 12A is side cross-sectional view of the apparatus of FIG. 1A, depicting a drive mechanism of the apparatus;

FIGS. 12B-12D are side cross-sectional views illustrating operation of the drive mechanism of FIG. 12A;

FIG. 13 is an exploded view of a locking assembly portion of the apparatus of FIG. 8;

FIG. 14A is an enlarged, front perspective view of the locking assembly of FIG. 13 upon final assembly;

FIG. 14B is an enlarged, rear perspective view of the locking assembly of FIG. 14A in a released position;

FIG. 15 is a cross-sectional view of a portion of the locking assembly of FIG. 13;

FIGS. 16A-16C are rear views illustrating operation of a selector cap component of the locking assembly of FIG. 13;

FIGS. 16D and 16E are rear perspective views of a portion of the apparatus of FIG. 1A, illustrating operation of the locking assembly of FIG. 13;

FIG. 17 is an enlarged, front perspective view of the apparatus of FIG. 8 upon final assembly;

FIG. 18 is an enlarged side view of an interior portion of a nose component of the apparatus of FIG. 8;

FIGS. 19A and 19B illustrate a first disc body useful with an adjustment device of the apparatus of FIG. 1A;

FIGS. 20A and 20B illustrate a second disc body useful with the adjustment device of FIG. 1A;

FIGS. 21A and 21B illustrate a control ring useful with the adjustment device of FIG. 1A;

FIG. 22 is a perspective view of a portion of a housing of the apparatus of FIG. 1A;

FIGS. 23A-23E illustrate operation of the adjustment device of FIG. 1A;

FIG. 24A is a perspective view of a retainment clasp of the apparatus of FIG. 1A;

FIG. 24B is a front perspective view of the clasp of FIG. 24A assembled to other components of the apparatus of FIG. 1A;

FIG. 25A is a side perspective view of a transfer cap useful with or as part of the apparatus of FIG. 1A;

FIG. 25B is a side plan view of the transfer cap of FIG. 25A;

FIGS. 26A-26D illustrate assembly of the transfer cap of FIG. 25A with a remainder of the surgical connection apparatus of FIG. 1A;

FIG. 27A is an exploded view of another surgical connection apparatus in accordance with aspects of the present disclosure;

FIG. 27B is a side perspective view of the apparatus of FIG. 27A upon final assembly, with a portion of the housing removed;

FIG. 28 is an exploded view of a delivery assembly portion of the apparatus of FIG. 27A in conjunction with a self-closing clip;

FIG. 29A is a perspective view of a distal portion of an inner member of the delivery assembly of FIG. 28;

FIG. 29B is a transverse, cross-sectional view of the inner member of FIG. 29A;

FIG. 29C is a longitudinal, cross-sectional view of the inner member of FIG. 29A;

FIG. 30A is a perspective view of a distal portion of a retention member of the delivery assembly of FIG. 28;

FIG. 30B is a top view of the retention member of FIG. 30A;

FIG. 30C is an end view of the retention member of FIG. 30A;

FIG. 31A is a perspective view of a self-closing clip useful with the apparatus of FIG. 27A in an undeflected state;

FIG. 31B is a perspective view of the clip of FIG. 31A in an deflected state;

FIG. 32 is a perspective view illustrating assembly of the clip of FIG. 31A to the retention member of FIG. 30A;

FIG. 33 is a cross-sectional view of the delivery assembly of FIG. 28, including a retained clip;

FIG. 34A is a simplified, cross-sectional view of a tubular graft structure;

FIG. 34B is a cross-sectional view of the tubular graft structure of FIG. 34A following a bevel cut operation;

FIGS. 34C-34F illustrate mounting of the tubular graft structure of FIGS. 34A and 34B to the surgical connection apparatus of FIG. 27A;

FIGS. 35A-35D illustrate initial stages of an anastomosing procedure using the surgical connection apparatus of FIG. 27A in anastomosing a tubular graft structure to a second structure;

FIGS. 36A-36E illustrate completion of the anastomosing procedure of FIGS. 35A-35D;

FIGS. 37A-37D illustrate completion of an anastomosing procedure using the surgical connection apparatus of FIG. 1A;

FIG. 38A illustrates a tubular graft structure secured to a second structure following an anastomosis procedure in accordance with aspects of the present disclosure;

FIG. 38B illustrates an anastomosis relationship achieved using a previous clip deployment-type surgical connection apparatus;

FIG. 39 is a top view of a kit in accordance with aspects of the present disclosure;

FIG. 40A is a perspective view of a tray component of the kit of FIG. 39;

FIG. 40B is a top view of the tray of FIG. 40A;

FIG. 41 is a perspective view of a loading pin component of the kit of FIG. 39;

FIGS. 42A and 42B are perspective views of a guide piece component of the kit of FIG. 39;

FIG. 43 is a perspective view of a sizer used in evaluating a tubular graft structure;

FIG. 44 is a perspective view of the kit of FIG. 39 in combination with a tubular graft structure; and

FIG. 45 is an enlarged, perspective view of the guide piece of FIGS. 42 and 42B in combination with a portion of a surgical connection apparatus partially loaded with a tubular graft structure.

### DETAILED DESCRIPTION

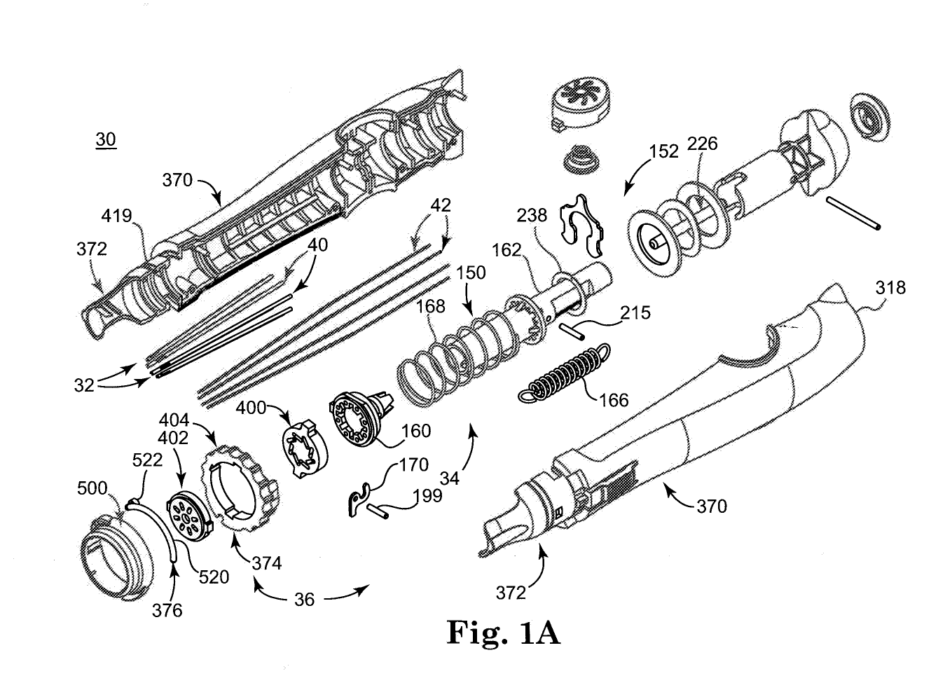

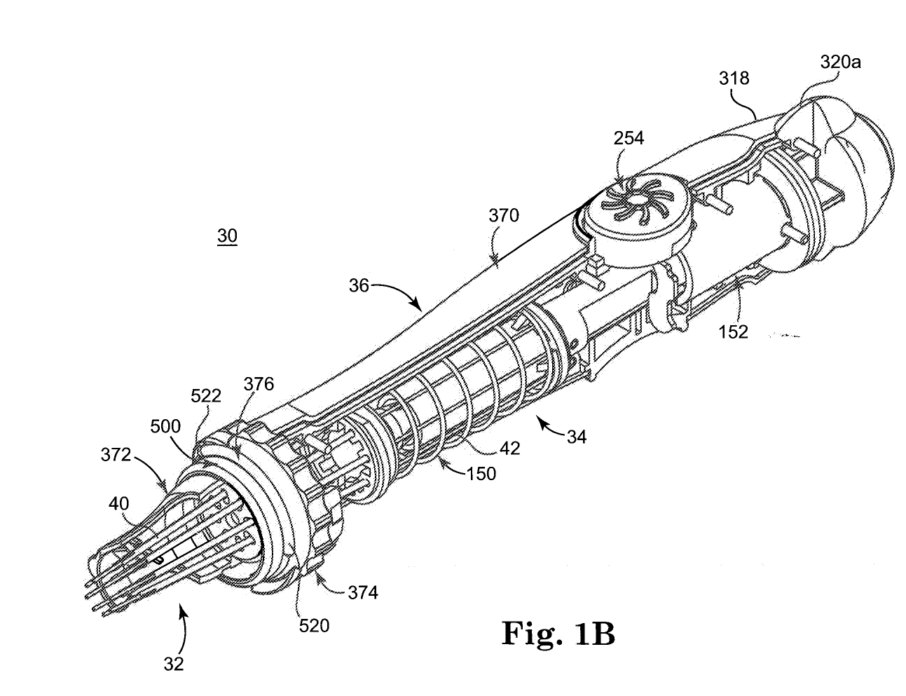

One configuration of a surgical connection apparatus 30 in accordance with aspects of the present disclosure is shown in FIGS. 1A and 1B. The apparatus 30 includes a plurality of delivery assemblies 32, a drive mechanism 34, and a housing assembly 36 (each referenced generally in FIGS. 1A and 1B). Details on the various components are provided below. In general terms, however, the housing assembly 36 maintains the delivery assemblies 32 and the drive mechanism 34. Each of the delivery assemblies 32 releasably maintains a self-closing clip (not shown). The drive mechanism 34, in turn, operates to effectuate substantially simultaneous release of each of the clips from corresponding ones of the delivery assemblies 32. With this construction, then, a tubular graft structure (not shown) can be mounted to the delivery assemblies 32 and subsequently secured to a second structure (not shown) via deployment of the clips in performing an anastomosis procedure.

The devices, systems, and methods described herein generally can be used to surgically connect structures in a patient. They can be used to connect or anastomose tubular structures or conduits together. The tubular structures can be vascular or non-vascular structures. The illustrative embodiments will be described in connection with coronary artery bypass grafting procedures during which a vascular conduit or tubular graft structure, such as a vein (e.g., a saphenous vein), artery (e.g., an internal mammary artery), or an artificial conduit or graft structure, is anastomosed to an aorta, the example second or target structure. It should be understood, however, that the principles disclosed herein can be used in other applications not specifically described. For example, the devices, systems, and methods can also be used to anastomose internal mammary arteries to coronary arteries, and saphenous veins to coronary, femoral, or popliteal arteries. As noted above, the devices described herein can also be used to connect other bodily lumens including non-vascular lumens, which can include, but are not limited to, the bile duct, the urethra, the urinary bladder, intestines, esophagus, stomach, and bowel.

### Delivery Assemblies 32

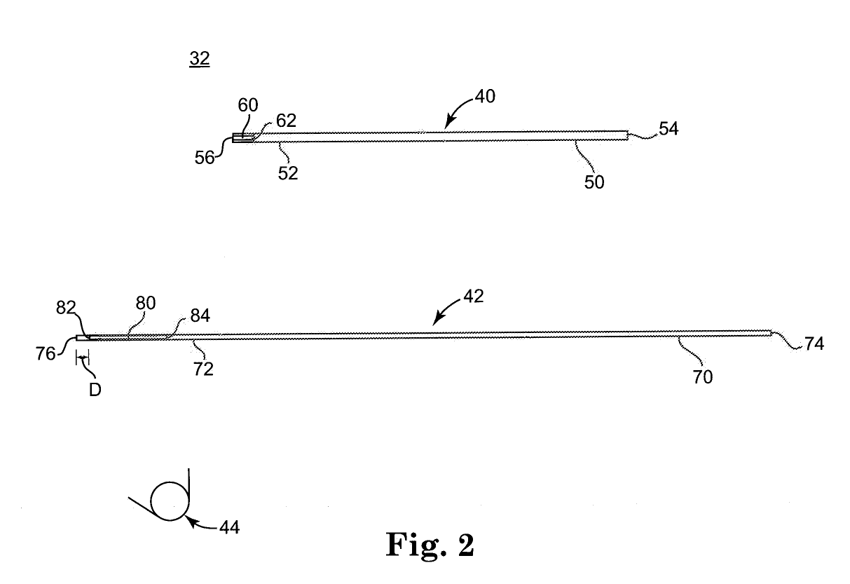

The apparatus 30 can include any number of the delivery assemblies 32 (e.g., 4, 6, 8, etc.), and the delivery assemblies 32 are, in some embodiments, identical. Regardless, FIG. 2 illustrates, in exploded form, one of the delivery assemblies 32 as including an outer member 40 and an inner member 42. In general terms, the outer member 40 is sized to slidably receive the inner member 42, with the members 40, 42 combining to capture and retain a self-closing clip 44 in a deflected state (it being understood that in the view of FIG. 2, the clip 44 is in an undeflected or “relaxed” state). Further, while the members 40, 42 are shown in FIG. 2 as being relatively straight, it will be understood from the following explanation that one or both of outer and inner members 40, 42 can have a pre-formed, intermediate curve or bend to facilitate mounting to the housing assembly 36 (or can be forced to assume a curved or bent shape upon final assembly).

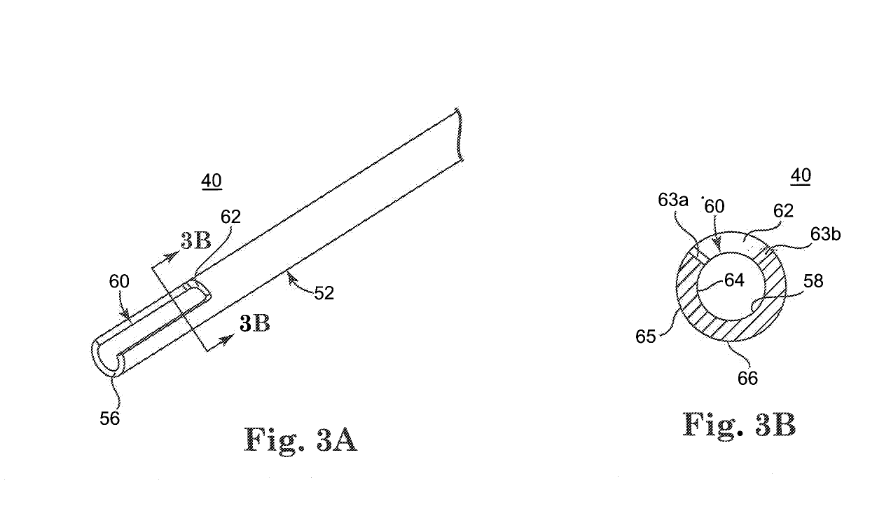

The outer member 40 is a tubular body (e.g., a sheath), and defines a proximal section 50 and a distal section 52. The proximal section 50 terminates at a proximal end 54, whereas the distal section 52 terminates at a distal end 56. With additional reference to FIGS. 3A and 3B, the outer member 40 is tubular, forming or defining a lumen 58. The lumen 58 is open relative to an exterior of the outer member 40 at both of the proximal and distal ends 54, 56. In addition, the outer member 40 forms an aperture 60 at the distal section 52, extending from the distal end 56. The aperture 60 provides a radial opening to the lumen 58 (as compared to a longitudinal opening provided at the distal end 56), and extends from the distal end 56 to a trailing edge 62. As described in greater detail below, the aperture 60 facilitates partial release of the clip 44 from the delivery assembly 32. For example, and as best shown in FIG. 3B, the aperture 60 is defined by opposing side walls 63a, 63b extending from an interior face or diameter 64 to an exterior face or diameter 65 of the outer member 40. Thus, a side 66 of the outer member 40 opposite the aperture 60 is exteriorly closed.

The outer member 40 can be formed of a relatively rigid material(s) capable of maintaining structural integrity when subjected to slight deflection forces, as well as sliding impingement of the clip 44 against the interior face 64. For example, in some embodiments, the outer member 40 is formed of stainless steel (e.g., 304 SST), although other materials are also acceptable. Various dimensions associated with the outer member 40 are selected to correspond with dimensions of the inner member 42 as described below. In general terms, however, the lumen 58 is sized to slidably receive the inner member 42, and thus can have a diameter on the order of 0.017-0.037 inch in some embodiments. With the one configuration of FIG. 2, a longitudinal length of the outer member 40 is less than that of the inner member 42, and can be on the order of 1.8-2.6 inches. A number of other dimensions, greater or lesser, are also acceptable. For example, where the surgical connection apparatus 30 is adapted for minimally invasive applications, the outer member 40 can have a length significantly greater than 2.6 inches. Finally, a length of the aperture 60 (i.e., dimension from the distal end 56 to the trailing edge 62) can be on the order of 0.07-0.17 inch and is otherwise selected to correspond with a length of the clip 44 in a deformed state, as well as expected location of the clip 44 within the inner member 42 as described below. In other embodiments, however, one or more other dimensions are also acceptable.

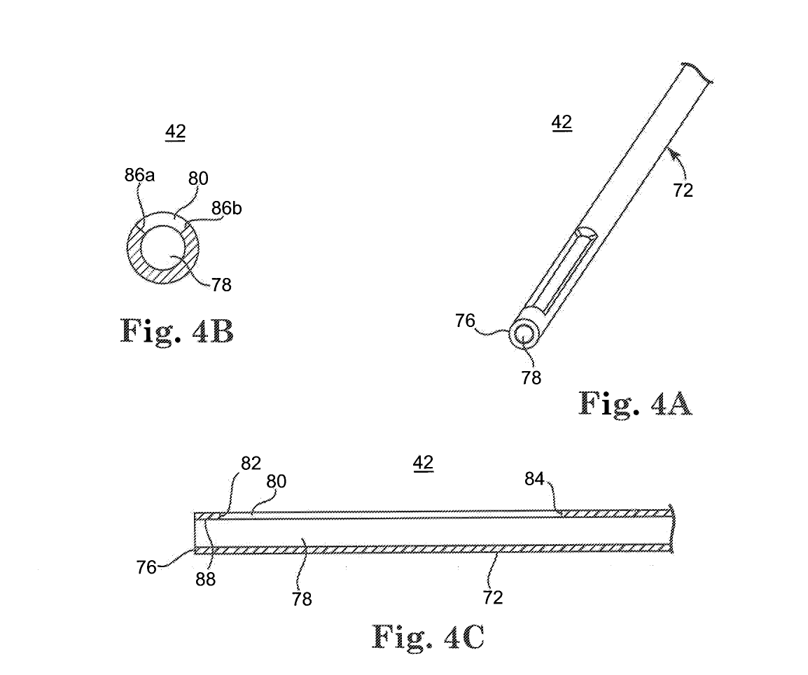

Returning to FIG. 2, the inner member 42 is an elongated body defining a proximal segment 70 and a distal segment 72. The proximal segment 70 terminates at a proximal end 74, whereas the distal segment 72 terminates at a distal tip 76. With additional reference to FIGS. 4A and 4B, the inner member 42 is at least partially tubular, and forms an internal passage 78. In some embodiments, the internal passage 78 extends from the distal segment 72 to the proximal segment 70. In other embodiments, the internal passage 78 is formed only along the distal segment 72, with the proximal segment 70 being akin to a solid shaft. Regardless, the internal passage 78 is sized to receive at least a portion of the clip 44 in a deflected state. Further, the internal passage 78 is exteriorly open relative to the inner member 42 via a longitudinal window 80. As described in greater detail below, the longitudinal window 80 facilitates release of the clip 44 from the internal passage 78. The window 80 is defined by opposed, first and second ends 82, 84, as well as opposed side walls 86a, 86b. The first end 82 is defined as being adjacent the distal tip 76, whereas the second end 84 is opposite (or proximal) the first end 82. With these conventions in mind, the first end 82 is proximally spaced from the distal tip 76. As best shown in FIG. 4C, then, the internal passage 78 extends distally beyond the first end 82 of the window 80, such that the inner member 42 provides an internal retention surface 88 distal the window 80. The retention surface 88 serves to selectively retain an end of the clip 44 (FIG. 2).

With the one embodiment of FIG. 4C, the internal passage 78 extends to and is longitudinally open at the distal tip 76 to facilitate loading of the clip 44 (FIG. 2) within the passage 48. In other embodiments, however, the internal passage 78 can be longitudinally closed at the distal tip 76 (so long as the retention surface 88 distal the window end 82 is still provided). Similarly, the distal tip 76 is shown in FIG. 4C as being relatively blunt; due to the highly thin nature of the inner member 42, the distal tip 76 will readily pierce through a tissue structure (e.g., a tubular graft structure). In other embodiments, however, the distal tip 76 can be sharpened or needle-like.

The inner member 42 can be an integral, homogenously-formed body, or can include two or more components assembled to one another. Regardless, the inner member 42 is formed of a relatively rigid, durable material such as stainless steel (e.g., 304 SST), plastic, etc.

Various dimensional characteristics of the inner member 42 are selected to correspond with those of the outer member 40 (FIG. 2) as well as to facilitate a desired clip deployment action. For example, the inner member 42 is sized to be slidably received within the outer member 40, and thus has an outer diameter less than an inner diameter of the outer member 40. For example, in some embodiments, the inner member 42 has an outer diameter in the range of 0.01-0.03 inch, and in other embodiments, 0.017-0.027 inch. Regardless, an outer diameter of the inner member 42 is preferably less than an inner diameter of the outer member 40 by a dimension on the order of 0.005 inch in some embodiments. An inner diameter of the inner member 42 (i.e., a diameter of the internal passage 78) is preferably sized to receive the clip 44 and thus can be, in some embodiments, on the order of 0.07-0.17 inch, although other dimensions are also acceptable.

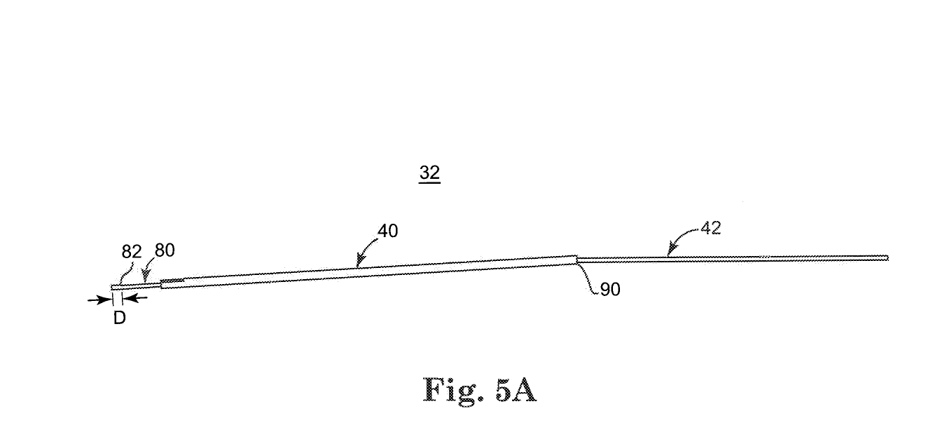

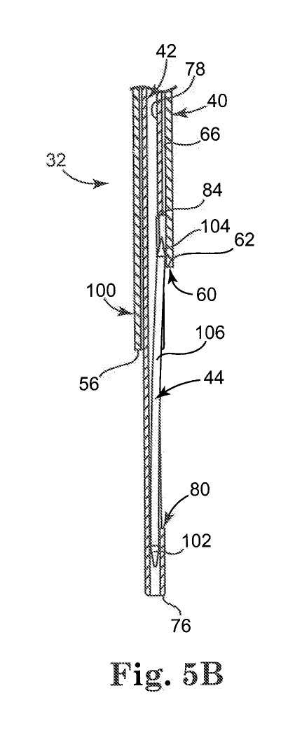

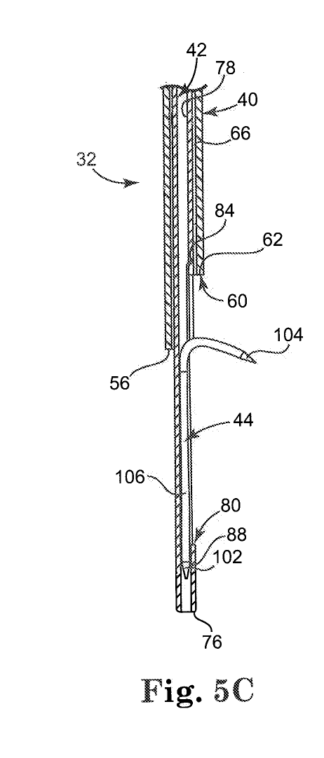

Assembly of the inner member 42 within the outer member 40 is shown in FIG. 5A and reflects that an overall length of the inner member 42 is, in some embodiments, greater than an overall length of the outer member 40. With this in mind, in some embodiments, the inner member 42 has a longitudinal length on the order of 3.5-4.5 inches and/or has a longitudinal length at least 1.5 inches greater than a longitudinal length of the outer member 40, although other dimensions are also acceptable (e.g., for minimally invasive applications, the inner member 42 can have a length significantly greater than 4.5 inches). Further, as shown in FIG. 5A, the inner member 42 can have a pre-formed curve or bend (generally indicated at 90 in FIG. 5A) along a length thereof corresponding with proximal extension from the outer member 40. In other embodiments, the inner member 42 will readily assume the shape (and thus curvature) of the outer member 40 upon placement therein or as dictated by subsequent assembly to the housing assembly 36 (FIG. 1A). Finally, the first end 82 of the window 80 is proximally spaced from the distal tip 76 by a distance D selected to facilitate mounting of a graft structure (not shown) onto the inner member 42 prior to deployment of the clip 44 (FIG. 2). With this in mind, in some embodiments, the distance D is on the order of 0.05-0.1 inch, and in other embodiments on the order of 0.07-0.08 inch, although other dimensions are also acceptable. Along these same lines, the window 80 can have a longitudinal length (i.e., distance between the first and second ends 82, 84 as best shown in FIG. 2) on the order of 0.3-0.6 inch, and in other embodiments on order of 0.4-0.5 inch, although other dimensions are also acceptable.

With the above features of the outer and inner members 40, 42 in mind, a distal region 100 of the delivery assembly 32 in a pre-deployment or “retained” state is shown in FIG. 5B, including the clip 44 retained thereby. The inner member 42 is slidably disposed within the outer member 40 such that the distal tip 76 of the inner member 42 extends distally beyond the distal end 56 of the outer member 40. A portion of the internal passage 78 is within the outer member 40, and is radially open relative to the outer member 40 via the window 80. In this regard, the outer and inner members 40, 42 are arranged relative to one another such that the window 80 of the inner member 42 “faces” the aperture 60 of the outer member 40. Finally, a longitudinal position of the inner member 42 relative to the outer member 40 is such that the second end 84 of the window 80 is located within the outer member 40, and is proximal the aperture 60 (and in particular the trailing edge 62 thereof).

Given the above conventions, the clip 44 is retained within the distal region 100 of the delivery assembly 32. As a point of reference, the delivery assembly 32 is transitionable from the retained state (as shown in FIG. 5B) in which the clip 44 is entirely captured by the delivery assembly 32, to a released state (FIG. 5C) in which the clip 44 can be deployed from the delivery assembly 32. Relative to the retained state of FIG. 5B, the clip 44 is arranged so as to define opposing, first and second ends 102, 104, and is forced to a deflected state within the delivery assembly 32. More particularly, the first end 102 is captured within the internal passage 78 of the inner member 42, for example via the first end 102 bearing against the retention surface 88. Thus, the first end 102 of the clip 44 is adjacent the distal tip 76 of the inner member 42. Conversely, the second end 104 of the clip 44 is proximal the first end 102, projects through the window 80 of the inner member 42, and is slidingly captured against or relative to the outer member 40 as shown (e.g., the second end 104 impinges against the interior face 64 of the outer member 42). As described below, the clip 44 is stressed to the deflected orientation of FIG. 5B, such that an intermediate portion 106 thereof bears against the inner member 42, forcing or biasing the ends 102, 104 to the positions shown in FIG. 5B.

The clip 44 can be deployed or released from the delivery assembly 32 upon transitioning of the delivery assembly 32 from the retained state of FIG. 5B to the released state of FIG. 5C. In particular, and is reflected by a comparison of FIGS. 5B and 5C, the inner member 42 is moved distally relative to the outer member 40 such that the second end 84 of the window 80, as well as the second end 104 of the clip 44, is aligned with or distally beyond the trailing edge 62 of the aperture 60. In this position, the second end 104 is no longer retained by the outer member 40. A shape memory characteristic of the clip 44 thus causes the clip 44 to naturally revert back toward an undeflected or relaxed state such that the second end 104 naturally deploys through the aperture 60, with an immediately adjacent portion of the clip 44 deploying from the internal passage 78 of the inner member 42 via the window 80 and the aperture 60. In some embodiments, the inner member 42 is configured such that even with partial release of the second end 104 through the aperture 60 (i.e., the arrangement of FIG. 5C), the first end 102 remains relatively rigidly engaged or captured within the internal passage 78 (for example via a wedged engagement of the first end 102 against the interior surface 88). With this but one acceptable configuration, the first end 102 can subsequently release from the inner member 42 via interface of the clip 44 with various structure(s) being anastomosed/secured as described below.

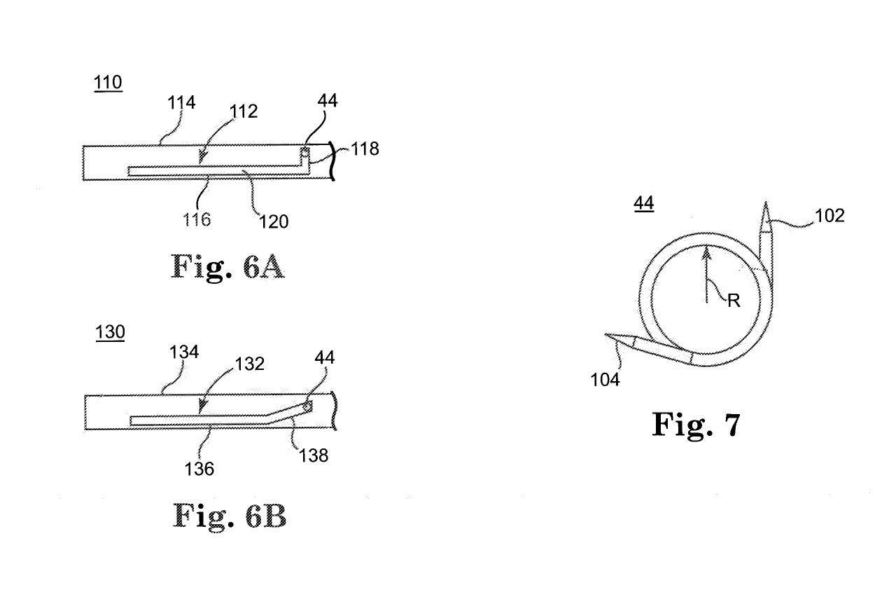

While operation of the delivery assembly 32 in capturing and subsequently releasing the clip 44 has been described with reference to the window 80 of the inner member 42 being substantially linear, in other embodiments, the window 80 can assume other configurations. For example, FIG. 6A illustrates, in simplified form, a portion of an alternative inner member 110 including a window 112 formed at a distal segment 114 thereof. The window 112 includes first and second sections 116, 118 each having a width sufficient to permit passage of the clip 44 therethrough. With this in mind, the second section 118 is substantially perpendicular to the first section 116. With this configuration, then, the clip 44 can partially extend from an internal passage 120 (referenced generally) of the inner member 110 via the window section 118 in a retained state of the corresponding delivery assembly (not shown) such that the second end 104 (FIG. 5B) of the clip 44 is external the inner member 110 (and captured by the outer member 40 (FIG. 2)). Once the second end 104, otherwise extending through the second section 118, is “exposed” relative to the corresponding outer member (i.e., with distal movement of the inner member 110 relative to the outer member as previously described), the inner member 110 is rotated to guide the clip 44 from the second section 118 to the first section 116, at which the clip 44 can entirely be released from the inner member 110.

Along these same lines, a portion of yet another alternative embodiment inner member 130 is shown in simplified form in FIG. 6B, and includes a window 132 formed at a distal segment 134 thereof. The window 132 includes a first section 136 and a second section 138, with the second section 138 extending proximally from the first section 136 at a non-perpendicular angle. With this configuration, in a retention state of the corresponding delivery assembly (not shown), the second end 104 (FIG. 5B) of the clip 44 extends through the second section 138. During a release operation, as the inner member 130 is moved to position the clip end 104 distally beyond the corresponding outer member 40 (FIG. 2), the user rotates the inner member 130 relative to the outer member 40, effectively guiding the clip 44 from the second section 138 to the first section 136, thus allowing full release or deployment of the clip 44 from the inner member 130.

As should be clear from the above, a wide variety of other inner member window configurations are also acceptable. Alternatively or additionally, the delivery assembly 32 (FIG. 2) can include other features and/or components that assist in effectuating deployment of the clip 44 from the internal passage 78 (FIG. 4B) of the inner member 42 (FIG. 2) such as, for example, biasing device(s) (e.g., spring(s), push rods, etc.).

Returning to FIG. 2, regardless of an exact configuration of the delivery assembly 32, operation of the delivery assembly 32 in retaining and subsequently deploying or releasing the clip 44 is, in some embodiments, premised upon a shape memory attribute of the clip 44. With additional reference to FIG. 7 (that otherwise illustrates one non-limiting example of the clip 44 in a natural or undeformed state), the clip 44 exhibits a loop-shaped memory set shape or configuration, which although shown as an overlapping loop in FIG. 7, can be non-overlapping or otherwise shaped differently than that shown. The clip 44 can be Nitinol wire and provided with the desired memory set configuration to exhibit pseudo elastic (super elastic) behavior. In other words, at least a portion of the shape memory alloy is converted from its austenitic phase to its martensitic phase, when the wire is in its deformed configuration. As the stress is removed, the material undergoes a martensitic to austenitic conversion and springs back (e.g., self-reverts) to its original undeformed or undeflected configuration.

The clip/wire 44 can have a variety of cross-sectional diameters and/or lengths appropriate for a particular surgical application. In some embodiments, the clip 44 has a diameter on the order of 0.001-0.015 inch and, in the undeflected or relaxed state of FIG. 7, a loop radius R on the order of 0.06-0.075 inch. Conversely, in the deflected state (e.g., FIG. 5B), the clip 44 has an overall length on the order of 0.4-0.45 inch, although other dimensions are equally acceptable. More particularly, dimension(s) of the clip 44 in either of the undeflected or deflected states and/or diameter can be selected to meet the requirements of any particular procedure with which the clip 44 is to be employed. Finally, the first and second ends 102, 104 of the clip 44 can be sharpened to promote piercing of target tissue (not shown). Alternatively, one or both of the ends 102 and/or 104 can be blunt.

### Drive Mechanism 34

Returning to FIGS. 1A and 1B and with the above understanding of the delivery assemblies 32 in mind, the drive mechanism 34 can assume a variety of forms configured to facilitate substantially simultaneous release of the clips (not shown) from respective ones of the delivery assemblies 32 by effectuating simultaneous, sliding movement of the respective inner members 42. In some embodiments, the drive mechanism 34 includes a drive assembly 150 and a locking assembly 152. In general terms, the drive assembly 150 is adapted to move the inner members 42 relative to the corresponding outer members 40 in a sliding fashion to effectuate release of the retained clips 44 (FIG. 2). The locking assembly 152, in turn, is adapted to retain the drive assembly 150 in a desired state prior to use, as well as to initiate operation of the drive assembly 150 in response to user actuation.

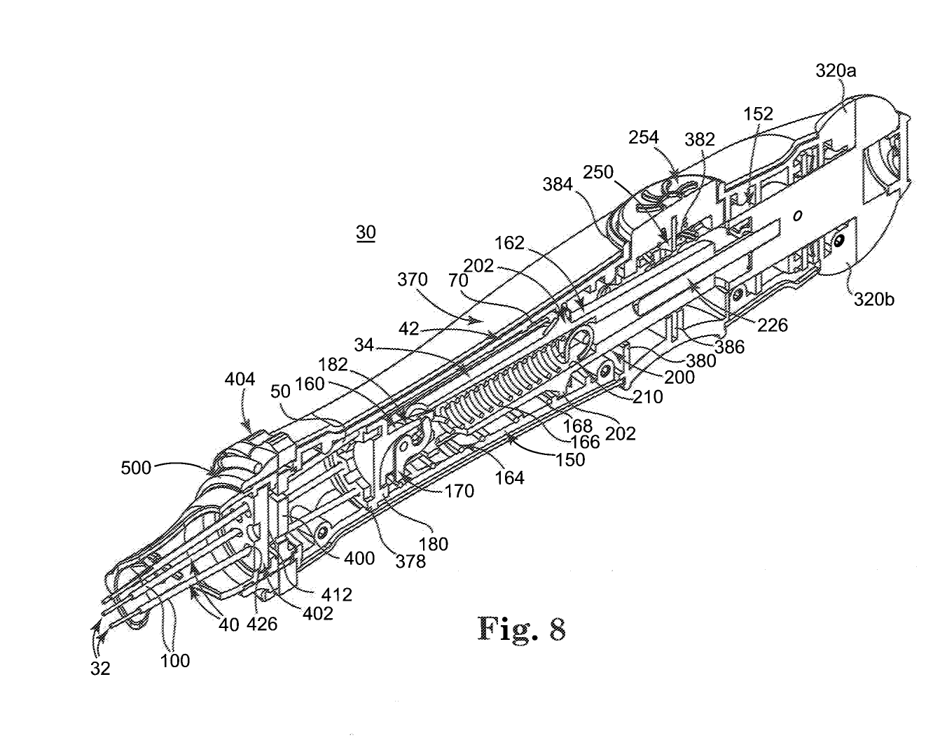

The drive assembly 150 is shown in greater detail in FIG. 8 and includes a first holder body 160, a second holder body 162, and a biasing assembly 164 (referenced generally) that biases the second holder body 162 relative to the first holder body 160 in a desired fashion. In this regard, and in some embodiments, the biasing assembly 164 can include first and second springs 166, 168, and a latch 170.

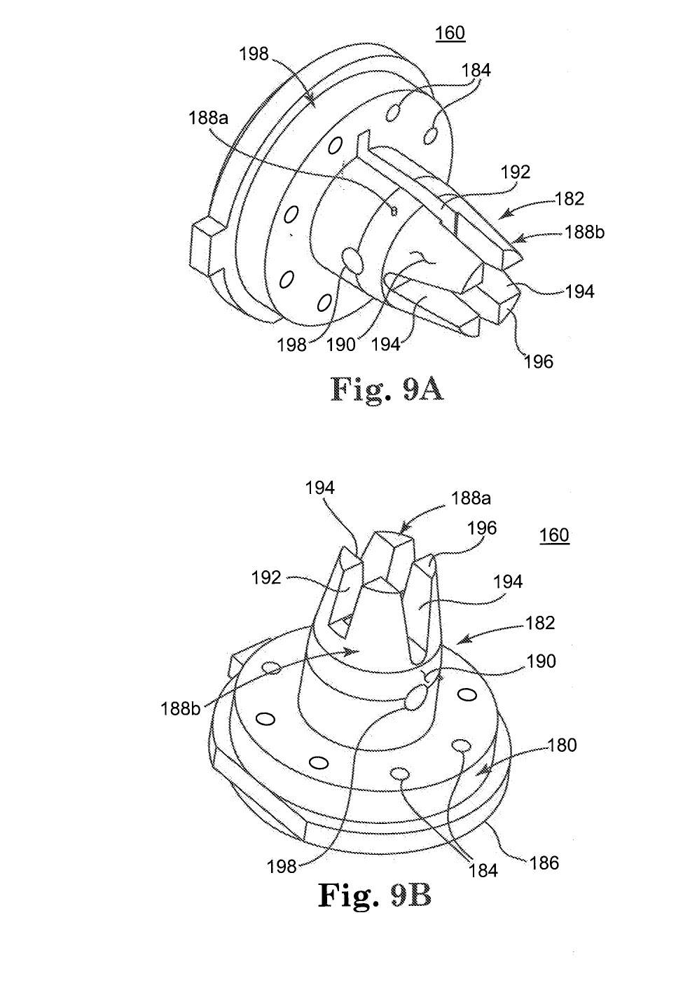

With reference to FIGS. 8, 9A, and 9B, the first holder body 160 includes, in some embodiments, a base 180 and a neck 182. The base 180 is adapted to rigidly retain respective ones of the outer members 40 (the proximal section 50 of several of which are shown in FIG. 8), as well as permit sliding movement of the inner members 42 (the proximal segment 70 of one of which is visible in FIG. 8) relative to the corresponding outer members 40. With this in mind, the base 180 can form a plurality of holes 184 each having a diameter slightly greater than an outer diameter of the inner members 42 such that each of the inner members 42 are slidable relative to a corresponding one of the holes 184. Similarly, respective ones of the outer members 40 can be coaxially disposed within a respective one of the holes 184, can be affixed (e.g., bonded, mechanically fixed, etc.) to a trailing face 186 of the base 180 in coaxial alignment with a respective one of the holes 184, etc. The holes 184 can be equidistantly spaced from one another, defining a circular pattern (that in turn is reflected in one or more other components of the apparatus 30 as described below), or can have differing relationships. Regardless, the base 180 is adapted to maintain a spatial position of the outer members 40 while permitting sliding movement of the corresponding inner members 42 relative to the outer members 40.

The neck 182 extends from the base 180 opposite the trailing face 186 and is, in some embodiments, adapted to interface with the second holder body 162 and the biasing assembly 164 as described below. With this in mind, the neck 182 can assume a variety of configurations, and includes two fingers 188a, 188b. As best shown in FIG. 9A, the fingers 188a, 188b combine to define an outer diameter of the neck 182 that tapers in extension from the base 180. That is to say, an outer surface 190 collectively formed by the fingers 188a, 188b tapers radially inwardly in extension of the neck 182 from the base 180. In addition, the fingers 188a, 188b are spaced from one another to define a gap 192. As made clear below, the gap 192 is sized to rotatably receive the latch 170 (FIG. 8), and can extend through all or part of the base 180 (e.g., the gap 192 can extend an entire length of the first finger 188a and into the base 180, and only along a partial length of the second finger 188b). Further, the fingers 188a, 188b each form a notch 194 that is open at a leading end 196 of the neck 182, and is sized to permit clearance of a portion of the first spring 166 (FIG. 8). Finally, a common bore 198 extends transversely through the fingers 188a, 188b and is sized to receive a pin 199 (FIG. 1A) employed to facilitate rotatable mounting of the latch 170 between the fingers 188a, 188b. Alternatively, however, the neck 182 can have a wide variety of other constructions apart from the configuration shown.

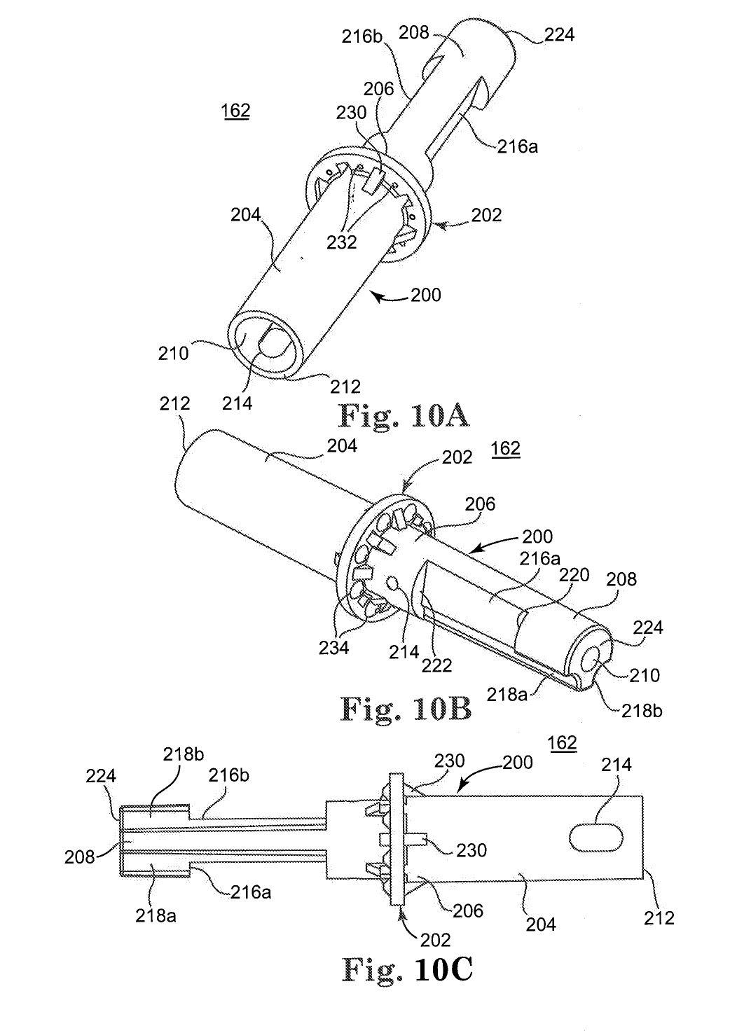

Returning to FIG. 8, and with additional reference to FIGS. 10A-10C, the second holder body 162 is adapted to maintain, and dictate movement of, the inner members 42, as well as to interface with the biasing assembly 164 as described below. With this in mind, in some embodiments, the second holder body 162 includes a central shaft 200 and a flange 202. In general terms, the shaft 200 defines a leading portion 204, an intermediate portion 206, and a trailing portion 208. The flange 202 extends radially from the shaft 200 along the intermediate portion 206.

As shown, the shaft 200 can be akin to a tube, defining a passage 210 (best shown in FIG. 8) between and through the leading and trailing portions 204, 208. Relative to the leading portion 204, the passage 210 is sized to receive the first spring 166 as described below, and is longitudinally open at a leading end 212 of the shaft 200. In this regard, a diameter of the passage 210 at the leading portion 204 is slightly greater than a diameter of the neck 182 of the first holder body 160, such that the leading portion 204 can coaxially receive the neck 182. In some embodiments, the leading portion 204 further forms an aperture 214 adjacent the leading end 212 that is sized to allow movement of the latch 170.

The intermediate portion 206 is adapted to facilitate mounting of the first spring 166 within the passage 210. Thus, in some embodiments, the intermediate portion 206 can include a radial bore 214 (FIG. 10B) sized to receive a pin 215 (FIG. 1A) otherwise facilitating assembly of the first spring 166 to the second holder body 162. Alternatively, however, a wide variety of other mounting techniques are also acceptable.

The trailing portion 208 extends from the intermediate portion 206, and in some embodiments is adapted to interface with the locking assembly 152 as described below. In general terms, the trailing portion 208 can form first and second grooves 216a, 216b and first and second channels 218a, 218b at opposite sides thereof (best shown in FIGS. 10B and 10C). The groove/channel pairs 216a/218a, 216b/218b can be identical such that the following description of the first groove 216a/first channel 218a of FIG. 10B is equally applicable to the second groove 216b/second channel 218b. The groove 216a extends between opposed shoulders 220, 222 that otherwise provide engagement surfaces for interfacing with a corresponding component of the locking assembly 152 (FIG. 8). The channel 218a is a continuation of, and extends distally from, the groove 216a, and is adapted to facilitate sliding movement of the second holder body 162 relative to a corresponding component of the locking assembly 152 as described below. Finally, in some embodiments, the passage 210 extends through the trailing portion 208 (as reflected in FIG. 10B) to a trailing end 224 thereof, defining a diameter sized to slidably receive a dowel 226 (FIGS. 1A and 8). The dowel 224 slidably interconnects the second holder body 162 with a corresponding component of the locking assembly 152, thus maintaining a centered relationship of the second holder body 162 during movement thereof, as described below. Alternatively, the second holder body 162 can be configured to interface with the locking assembly 152 in a number of different manners.

As indicated above, the flange 202 is formed along the intermediate portion 206, and extends radially outwardly from the shaft 200. An outer diameter defined by the flange 202 is generally sized in accordance with the second spring 168 such that when the leading portion 204 is co-axially disposed within the second spring 168, an end thereof abuts against the flange 202. In this regard, the second holder body 162 can include one or more support struts 230 proximal the flange 202 that promote centering of the second spring 168 about the shaft 200. In addition, the flange 202 forms bores 232 (FIG. 10A) sized to receive respective ones of the inner members 42. The bores 232 can each be distally closed by a grommet 234 (FIG. 10B) or similar component that promotes a secure affixment between the corresponding inner member 42 and the flange 202. Finally, the second holder body 162 can include tines 236 distal the flange 202. The tines 236 are configured to retain a split ring 238 (FIG. 1A) otherwise employed to further secure or reinforce the grommets 234 to respective ones of the inner members 42. Alternatively, the second holder body 162 can assume a variety of other forms.

With specific reference to FIG. 8, upon final assembly, the outer members 40 are affixed to the first holder body 160, whereas the inner members 42 are affixed to the second holder body 162. The second holder body 162 is positioned proximal the first holder body 160, with the neck 182 of the first holder body 160 being coaxially positioned within the passage 210 of the second holder body 162. Further, the dowel 226 maintains an axial relationship of the second holder body 162 relative to the first holder body 160. As a point of reference, FIG. 8 represents an arrangement of the drive assembly in a loaded state (i.e., prior to deployment of the clips 44 (FIG. 2) from the delivery assemblies 32). With this arrangement, then, as the second holder body 162 is moved in a distal fashion, toward the first holder body 160 (i.e., leftward relative to an orientation of FIG. 8), the first holder body 160 maintains a spatial position of the outer members 40, whereas the second holder body 162 distally slides the inner members 42 through or relative to the corresponding outer members 40. In other words, the drive assembly 150 effectuates deployment of the clips 44 (FIG. 2) in transitioning from the loaded state of FIG. 8 to a deployed state in which the second holder body 162 moves distally toward the first holder body 160. In this regard, the biasing assembly 164 causes the above movement to occur.

More particularly, in one embodiment the first spring 166 is a coiled extension spring mounted to the neck 182 of the first holder body 160 and the shaft 200 of the second holder body 162. In the loaded state of FIG. 8, the first spring 166 is under tension, exerting a biasing force on the second holder body 162 toward the first holder body 160. As a point of reference, the locking assembly 152 retains the positions of the first and second holder bodies 160, 162 in the orientation of FIG. 8; upon actuation or release of the locking assembly 152, the first spring 166 then serves to bias the first holder body 160 toward the second holder body 162.

In some embodiments, the biasing assembly 164 can consist solely of the first spring 166 that otherwise biases the second holder body 162 toward the first holder body 160 when the second holder body 162 is released by or from the locking assembly 152. It will be understood, however, that this motion results in the inner members 42 being maintained in an overtly extended position relative to the corresponding outer member 40 following “firing” of the drive assembly 150. In some instances, it may be desirable to automatically retract the inner members 42 relative to the corresponding outer members 40 in connection with an anastomosis procedure. The biasing assembly 164 can thus be adapted to facilitate this optional performance characteristic via the second spring 168 and the latch 170.

In particular, the second spring 168 is a compression spring disposed and bearing against the base 180 of the first holder body 160 and the flange 202 of the second holder body 162. A spring force constant of the second spring 168 is less than that of the first spring 166 such that relative to the loaded state of FIG. 8, the retraction force exerted by the first spring 166 in pulling the second holder body 162 toward the first holder body 160 is greater than an extension force exerted by the second spring 168 in biasing the second holder body 162 away from the first holder body 160. In other words, the springs 166, 168 are configured such that the second spring 168 biases the second holder body 162 away from the first holder body 160 only in the absence of the first spring 166.

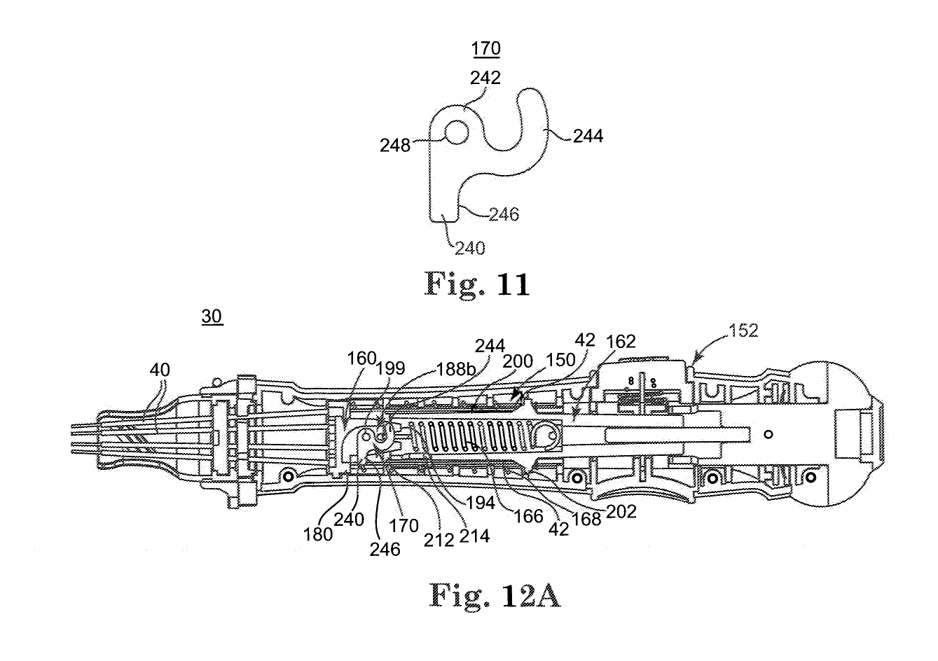

Given the above spring force characteristics associated with the springs 166, 168, the latch 170 releasably connects the first spring 166 to the first holder body 160 such that the force applied to the holder bodies 160, 162 by the first spring 166 can be removed via operation of the latch 170. For example, and with additional reference to FIG. 11, the latch 170 can include a foot 240, a central portion 242, and an arm 244. The foot 240 and the arm 244 extend in a generally opposed fashion from the central portion 242. Further, the foot 240 is sized to, upon final assembly, interface with the shaft 200, and defines a forward surface 246. The arm 244, in turn, is adapted to releasably engage an end of the first spring 166. Finally, the central portion 242 is adapted for rotatable mounting to the first holder body 160, for example by forming a hole 248. In particular, the latch 170 has a thickness less than a size of the gap 192 (FIG. 9A) defined between the fingers 188a, 188b (FIG. 9A). Thus, the latch 170 is positioned within the gap 192, with the central portion 242 being rotatably mounted to the fingers 188, for example, via the pin 199 (FIG. 1A) otherwise extending through the common bore 198 (FIG. 9A) and the hole 248.

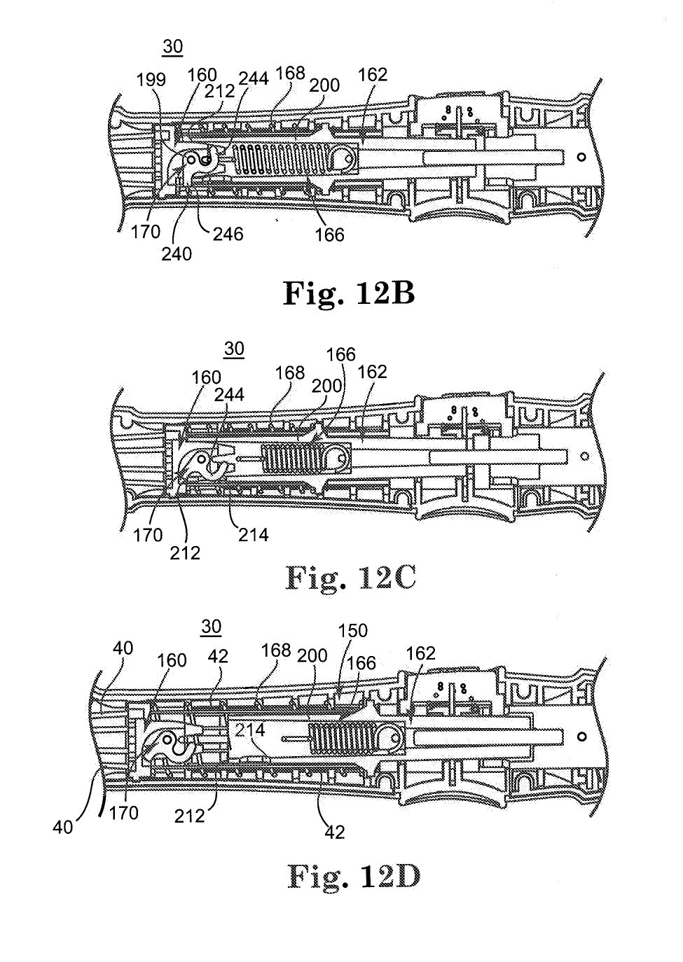

With the above construction, then, in the loaded state of FIG. 12A, the first spring 166 is connected to the first holder body 160 via the latch 170, as well as the second holder body 162 as previously described. The notch 194 of the second finger 188b (it being understood that the first finger 188a is not visible in the view of FIG. 12A) provides for clearance of the first spring 166 as assembled to the latch 170. In addition, the second spring 168 is mounted to the base 180 of the first holder body 160 and the flange 202 of the second holder body 162. During use, as the drive assembly 150 is released from the loaded state of FIG. 12A (e.g., via actuation of the locking assembly 152 as described below), the first spring 166 biases the second holder body 162 distally toward the first holder body 160 (it being recalled that the “contraction” spring force constant of the first spring 166 is greater than the “expansion” spring force constant of the second spring 168).

Distal movement of the second holder body 162 toward the first holder body 160 continues until the leading end 212 of the shaft 200 contacts the forward surface 246 (and thus the foot 240) of the latch 170 as shown in FIG. 12B. With continued distal movement of the second holder body 162 against the foot 240, the latch 170 is caused to rotate, pivoting about the pin 199 (clockwise relative to the orientation of FIG. 12B). Further distal movement of the second holder body 162, and thus of the shaft 200, further rotates the latch 170 to a released state as shown in FIG. 12C. In the released state, the arm 244 has rotated away or disengaged from the first spring 166, such that the first spring 166 is no longer coupled by the latch 170 between the first and second holder bodies 160, 162. To facilitate this desired rotation of the latch 170, the aperture 214 provides necessary clearance of the arm 244 relative to the shaft 200.

Upon release of the first spring 166 from the latch 170, the contraction force exerted by the first spring 166 is removed. As a result, the expansion force generated by the second spring 168 freely biases the second holder body 162 proximally away from the first holder body 160, causing the second holder body 162 to move away from the first holder body 160 and thus retracting the inner members 42 relative to the corresponding outer members 40. The retracted state of the drive assembly 150 is shown in FIG. 12D.

Although the drive assembly 150 has been described as automatically facilitating both extension and subsequent retraction of the inner members 42 relative to the corresponding outer members 40, in other embodiments, only the extension motion need be provided. Thus, the drive assembly 150 can assume a wide variety of other forms apart from those specifically shown.

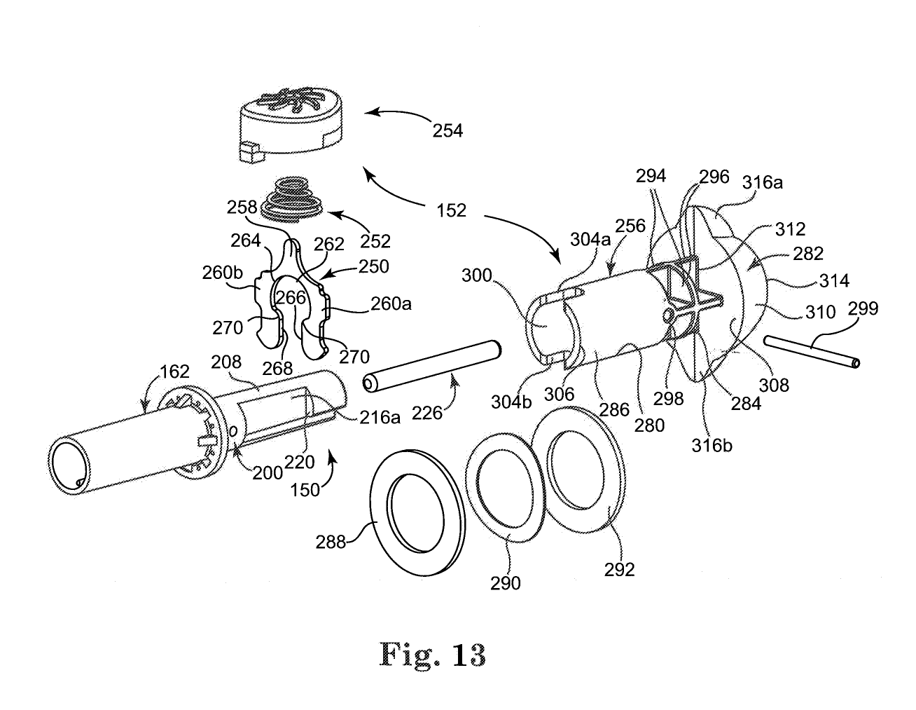

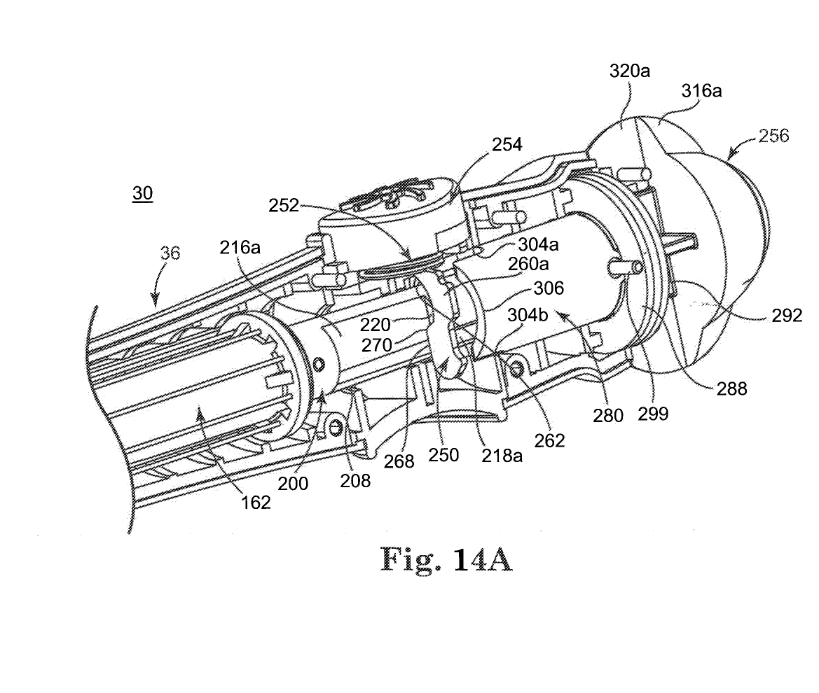

As mentioned above, the locking assembly 152 serves to retain the drive assembly 150 in the loaded state of FIG. 8. With this in mind, one embodiment of the locking assembly 152 is shown in greater detail in FIG. 13, along with related components of the drive assembly 150 (e.g., the second holder body 162 and the dowel 226). The locking assembly 152 includes a plunger body 250, a biasing device 252, an actuator 254, and an optional selector cap 256. In general terms, the plunger body 250 interfaces with the trailing portion 208 of the second holder body 162 (such that features of the trailing portion 208 can be considered part of the locking assembly 152), and is connected to the actuator 254. The biasing device 252 biases the plunger body 250/actuator 254 to a raised or locked position. The selector cap 256 selectively permits depression of the actuator 254. When the selector cap 256 is properly aligned with the actuator 254 and a user-applied force sufficient to overcome a bias of the biasing device 252 is placed upon the actuator 254, the plunger body 250 transitions to a lowered or release position as described below.

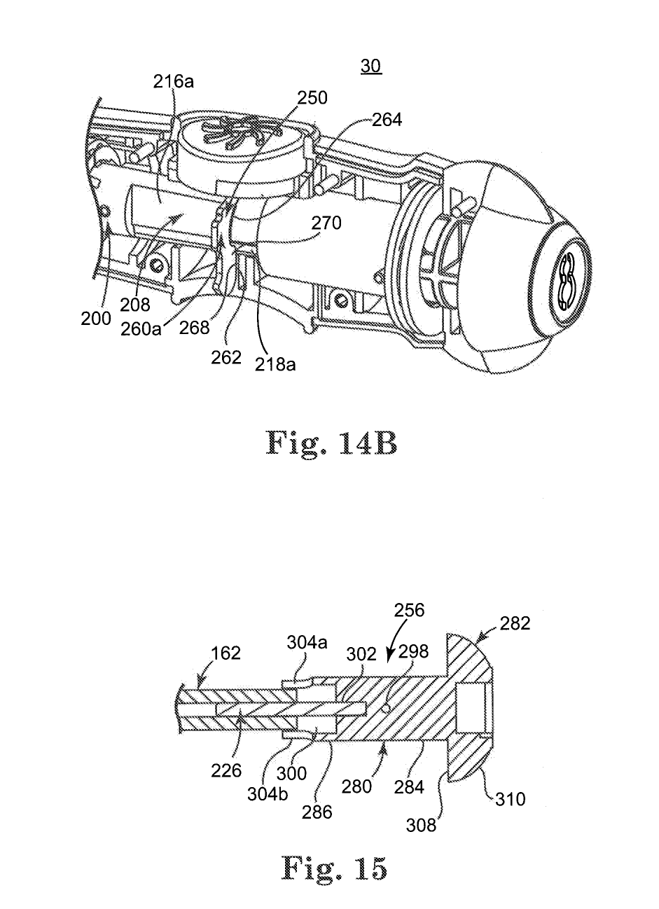

With additional reference to FIG. 14A, the plunger body 250 is slidably retained by the housing assembly 36 as described below, and includes a head 258 and opposing legs 260a, 260b. The head 258 is adapted for mounting to the actuator 254 as described below. The legs 260a, 260b extend from the head 258 in a symmetrical fashion, combining to define a recess 262 configured for selective clearance about the second holder body 162 upon final assembly. More particularly, the recess 262 includes a leading section 264 and a trailing section 266. The leading section 264 is adjacent the head 258, and is characterized by an increased width as compared to a width of the recess 262 along the trailing section 266. In this regard, each of the legs 260a, 260b forms or provides a stop surface or projection 268 along the trailing section 266, as well as a corner 270 in transitioning from the trailing section 266 to the leading section 264.

Dimensional features of the plunger body 250, and in particular the legs 260a, 260b and the recess 262, are selected in accordance with dimensional characteristics of the trailing portion 208 of the second holder body 162. More particularly, a width of the recess 262 at the trailing section 266 is commensurate with a width of the trailing portion 208 of the shaft 200 in a region of the grooves 216a, 216b (it being understood that the groove 216b being hidden in the view of FIG. 14A) such that in the raised position of FIG. 14A, the legs 260a, 260b (the leg 260b is hidden in FIG. 14A) nest within a corresponding one of the grooves 216a, 216b. Further, the stop surfaces 268 provided by the legs 260a, 260b bear or abut against a corresponding one of the shoulders 220 (it being understood that only one of the stop surfaces 268/shoulder 220 interfaces is visible in the view of FIG. 14A).

Conversely, the leading section 264 of the recess 262 is sized for clearance about the shaft 200. As shown in the lowered position of FIG. 14B, a size and shape of the leading section 264 is slightly greater than that of the trailing portion 208 of the shaft 200, such that the respective stop surfaces 268 are outside of the corresponding grooves 216a, 216b (it being understood that the second leg 260b and the second groove 216b are hidden in FIG. 14B). Further, the legs 260a, 260b are configured to interface with a corresponding one of the channels 218a, 218b (one of which is visible in FIG. 14B) of the shaft 200. More particularly, in the lowered position, the corner 270 of each of the legs 260a, 260b is slidably received in a corresponding one of the channels 218a, 218b, whereas a majority of the shaft 200 is slidably received within the leading section 264 of the recess 262.

The biasing device 252 and the actuator 254 combine to bias the plunger body 250 to the raised position of FIG. 14A. In particular, the head 258 of the plunger body 250 is mounted to the actuator 254, with the biasing device 252, such as a spring, being disposed between and bearing against the actuator 254 and a corresponding surface of the housing assembly 36. The actuator 254, in turn, is sized to be slidably captured by a corresponding feature of the housing assembly 36. With this arrangement, then, the biasing device 252 biases the actuator 254, and thus the plunger body 250, to the raised position of FIG. 14A. Conversely, when a sufficient force is placed upon the actuator 254, for example, by a user's thumb or finger, a force of the biasing device 252 is overcome and the plunger body 250 is moved to the lowered position described above, thus releasing the second holder body 162 from the locked state. In this regard, in one embodiment, the actuator 254 is a single button, and the locking assembly 152 is arranged such that the driving assembly 150 is “released” from the loaded state via movement of the actuator 254 in a direction generally perpendicular to a direction of subsequent movement of the second holder body 162 and thus of the inner members 42 (FIG. 1A). That is to say, unlike conventional surgical connection apparatus designs in which the user is required to effectuate an actuation motion that is generally parallel with subsequent delivery assembly movement, with the one embodiment of FIGS. 14A and 14B, a perpendicular actuator motion is established. As a result, a user can more easily effectuate clip deployment in an ergonomically beneficial manner. Alternatively, however, the locking assembly 152 can assume a number of other configurations differing from that shown and described.

In some embodiments, the selector cap 256 is included and serves to prevent inadvertent depression of the actuator 254 (and thus “firing” of the drive assembly 150) until such time as desired by a user. As shown in FIGS. 13 and 15, the selector cap 256 can include a stem 280 and a head body 282. The stem 280 is distally disposed relative to the head body 282, and includes first and second regions 284, 286. The first region 284 extends from the head body 282, and defines an outer circumference sized to co-axially receive one or more retention rings 288-292 (FIG. 13). The first region 284 can form or include recesses 294 separated by ribs 296 that facilitate co-axial placement of the retention rings 288-292 over the stem 280, as well as assembly to the head body 282. Further, the recess 294/rib 296 construction enhances manufacturability (molding) of the selector cap 256 by minimizing an overall mass. Alternatively, however, the first region 284 can be a more uniform cylinder. Regardless, in some embodiments, the first region 284 forms a bore 298 sized to receive an elongated pin 299 otherwise employed to capture the retention rings 288-292 relative to the stem 280 and within the housing assembly 36 as shown in FIG. 14A.

Returning to FIGS. 13 and 15, the second region 286 extends from the first region 284, and forms a central passageway 300. As shown in FIG. 15, the central passageway 300 is sized to receive a portion of the second holder body 162 and the dowel 226, and can include a stepped segment 302 adjacent the first region 284 at which the dowel 226 is rigidly affixed. In addition, the second region 286 forms or includes opposing slots 304a, 304b to permit movement therethrough of a portion of the actuator 254 as described below. With this configuration, the second region 286 provides a closed surface 306 (FIG. 13) circumferentially between the slots 304a, 304b.

The head body 282 extends from the stem 280 to define a face 308 and an exterior portion 310. The face 308 is configured for, upon final assembly, abutment against a corresponding surface(s) of the housing assembly 36 (FIG. 1A), and can include features for promoting connection to the stem 280 (e.g., the face 308 can form grooves 312 sized to frictionally receive the ribs 294 of the step 280); alternative forms of assembly of the step 280/head body 282 are also acceptable and the selector cap 256 can be integrally or homogenously formed.

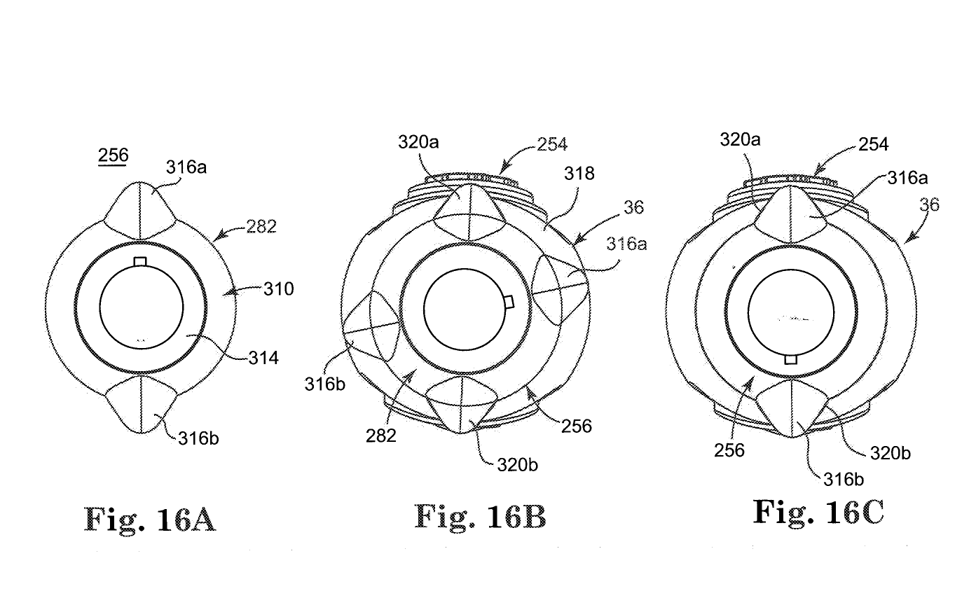

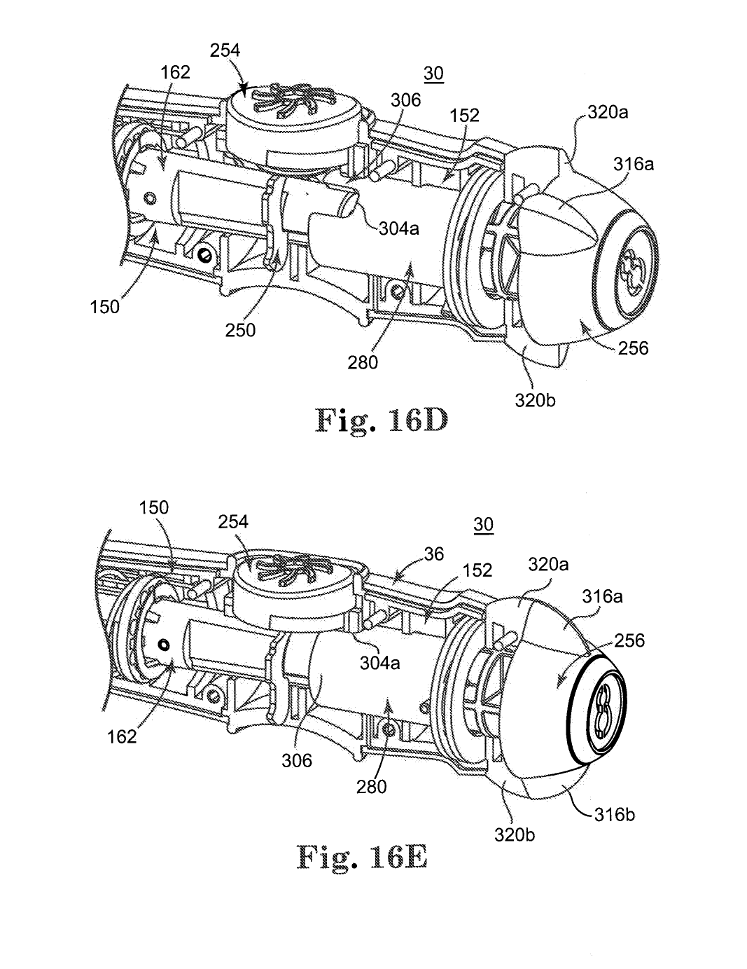

The exterior portion 310 includes or forms a central region 314 and indicator arms 316a, 316b. With additional reference to FIG. 16A, the indicator arms 316a, 316b extend in an opposing fashion from the central region 314, and are axially aligned with a respective one of the slots 304a, 304b. With this configuration, then, the indicator arms 316a, 316b provide an indication of a rotational/spatial position of the slots 304a, 304b. In some embodiments, and as shown in FIG. 16B, a proximal end 318 of the housing assembly 36 forms or defines one or more projection contours 320a, 320b, with one of the projection contours (e.g., the projection 320a) being axially aligned with the actuator 254.

With the above construction, during periods when actuation of the drive assembly 150 (FIG. 8) is not desired (e.g., when it is desired that inadvertent contact with the actuator 254 not cause “firing” of the drive assembly 150), the selector cap 256 is rotatably positioned such that the indicator arms 316a, 316b are not in alignment with the projection contours 320a, 320b (e.g., the arrangement of FIG. 16B). In this position and with additional reference to FIG. 16D, neither of the slots 304a, 304b (one of which is visible in FIG. 16D) are radially aligned with the actuator 254. Thus, if a depression or inward force is accidentally placed on the actuator 254, the stem 280 prevents the actuator 254 from moving inwardly due to contact between the actuator 254 and the closed surface 306. As a result, the actuator 254 cannot radially displace the plunger body 250 relative to the second holder body 162 in a manner otherwise “releasing” the second holder body 162 from the locked state.

Conversely, when operation of the drive assembly 150 (FIG. 8) is desired, the second holder body 162 can be released from the locked state by first rotating the selector cap 256 relative to the housing assembly 36 such that the indicator arms 316a, 316b are aligned with respective ones of the projection contours 320a, 320b as in FIG. 16C, for example. In this orientation, one of the slots 304a or 304b will be radially aligned with the actuator 254, as shown in FIG. 16E. A depression force subsequently placed on the actuator 254 will then cause the actuator 254 to move inwardly (and through the slot 304a or 304b), resulting in “release” or actuation of the drive assembly 150 as previously described. With this but one acceptable configuration, then, an overt, conscious action is required by the user (i.e., rotating the selector cap 256) before the drive assembly 150 can be actuated or “fired” (e.g., in some embodiments, the surgical connection apparatus 30 is initially provided to a user in the “locked” state, with the selector cap 256 rotated to the unaligned position of FIG. 16B). Alternatively, a wide variety of other configurations for the locking assembly 152 are also available that may or may not include the selector cap 256 and related features.

### Housing Assembly 36

Returning to FIGS. 1A and 1B, the housing assembly 36 maintains the delivery assemblies 32 and the drive mechanism 34, and includes a housing 370, a nose 372, an optional adjustment device 374, and an optional retainment clasp 376. Details on the various components are provided below. In general terms, however, the housing 370 maintains the drive mechanism 34, portions of the delivery assemblies 32, the adjustment device 374, and the clasp 376. The nose 372 extends from the housing 370, and provides a guide surface for positioning of the apparatus 30 during an anastomosis procedure or other procedure. The adjustment device 374 is connected to the delivery assemblies 32 and facilitates arrangement of the delivery assemblies 32 at a desired radial spacing. Finally, the clasp 376 is pivotably secured to the housing 370 and facilitates selective securement of a graft structure (not shown, e.g., a vein) to the housing 370.

The housing 370 can assume a variety of shapes and sizes conducive for convenient handling by a surgeon. Further, the housing 370 can include or form various internal features that retain various components of the drive mechanism 34 in a manner permitting desired operation thereof. For example, the proximal end 318 can include the projection contour(s) 320a, 320b (FIG. 16B) as previously described. Also, and as shown in FIG. 8, the housing 370 can form an annular groove 378 adapted to capture the first holder body 160 in a non-movable fashion. Similarly, the housing 370 can include or form an internal, annular spacer 380 sized and positioned to slidably interface with the second holder body 162. Finally, the housing 370 can include an annular shelf 382 positioned adjacent a well 384. The well 384 is configured to capture the actuator button 254, and permit sliding movement thereof as previously described. Conversely, the support surface 382 slidably supports the trailing portion 208 of the shaft 200, and maintains the plunger body 250. For example, the support surface 382 can form a slot 386 within which the plunger body 250 is slidably disposed.

It will be understood that the various features 320a, 320b, and 378-386 described above represent but one acceptable configuration of the housing 370. Thus, where, for example, the drive mechanism 34 has a varying configuration, one or more of the features 320a, 320b, 378-386 can be modified and/or eliminated, or other internal features provided. For example, various other features of the housing 370 are described below relative to the adjustment device 374 (FIG. 1A). Regardless, the housing 370 can be a homogeneous, integrally formed body, or can include two or more portions (as reflected in FIG. 1A) that are separately formed and subsequently assembled. Along these same lines, while the housing 370 and the nose 372 are generally described herein as being discrete components, in other embodiments, the housing 370 and the nose 372 can be integrally formed (or, where the housing 370 consists of two or more portions, the nose 372 can similarly include a corresponding number of portions each integrally formed with a corresponding portion of the housing 370).

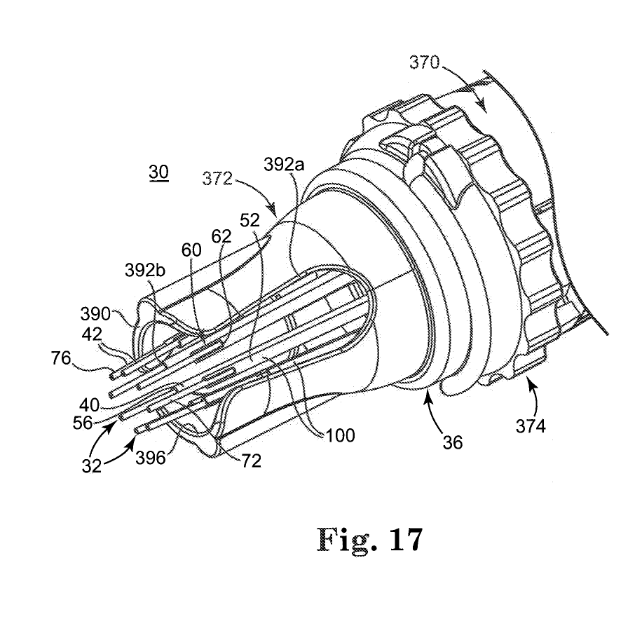

With reference to FIG. 17, the nose 372 extends distally from the housing 370, terminating at a contact surface 390. In general terms, the nose 372 is sized to axially encompass the delivery assemblies 32 otherwise projecting through the nose 372, with the contact surface 390 being spatially positioned relative to a distal extension of the delivery assemblies 32 to facilitate deployment of the clips 44 (FIG. 2) at a desired spatial position. More particularly, and as described in greater detail below, the contact surface 390 is spatially or longitudinally positioned relative to the delivery assemblies 32 such that when the contact surface 390 abuts a structure to which an anastomosis procedure is to be performed, the clips 44 will deploy from the delivery assemblies 32 into pierced engagement with the structure against which the contact surface 390 bears. For example, the housing assembly 36 can be configured such that upon final assembly of the apparatus 30, the contact surface 390 is approximately aligned with the distal end 56 of each of the outer members 40, whereas the distal tip 76 of each of the inner members 42 is distally beyond the contact surface 390. Regardless, the trailing side 62 of the aperture 60 formed by each of the outer members 40 is proximal the contact surface 390 so that initial clip deployment can occur proximal relative to a structure placed against the contact surface 390 via the aperture 60.

In some embodiments, the nose 372 forms one or more side openings 392a, 392b. The side openings 392a, 392b are sized to permit passage of a typical tubular graft structure (not shown) therethrough (and otherwise mounted to the delivery assemblies 32) as described below, as well as placement and/or removal of an optional transfer cap (not shown). In other embodiments, one or more of the side openings 392a, 392b can be eliminated.

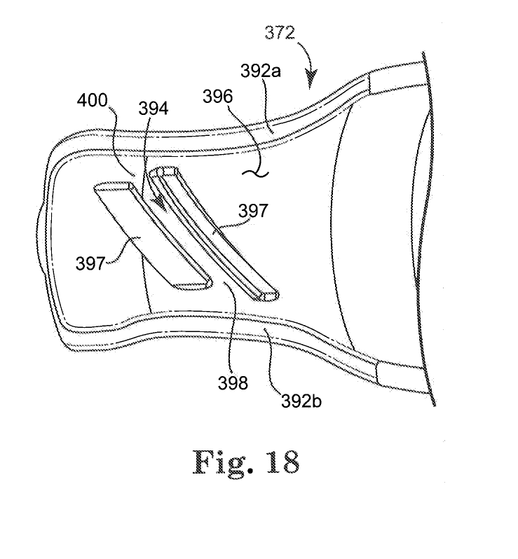

In some embodiments, the nose 372 includes or forms an additional feature or features that promote use of the optional transfer cap mentioned above. In general terms, and with reference to FIG. 18, a channel 394 can be defined along an interior surface 396 of the nose 372, for example by an opposing pair of thread bodies 397. The channel 394 is adjacent the side openings 392a, 392b (referenced generally), and is sized to frictionally engage a corresponding surface of the transfer cap. In this regard, the channel 394 extend in an angular fashion relative to a central axis of the nose 372 between a proximal end 398 and a distal end 400. As shown, the proximal end 398 is more closely adjacent the first side opening 392a, whereas the distal end 400 is more closely adjacent the second side opening 392b. As made clear below, this arrangement corresponds with a configuration of the transfer cap, and dictates desired assembly of the transfer cap relative to the nose 372 (e.g., the transfer cap can “enter” and “exit” the nose 372 via the first side opening 392a). Alternatively, the nose 372 can assume a variety of other configurations that may or may not include the channel 394.

Returning to FIG. 17, the housing assembly 36 and the drive mechanism 34 (FIG. 1A) are mounted to the delivery assemblies 32 such that the delivery assemblies 32 collectively define a relatively circular arrangement at distal regions thereof. As a point of reference, each of the delivery assemblies 32 can each be designated as defining the distal region 100 consisting of the distal section 52 of the outer member 40 (including the aperture 60) and the distal segment 72 of the inner member 42 (including the distal tip 76). The distal regions 100 extend from the housing 370 and collectively define the generally circular arrangement referenced above. An effective diameter of this circular arrangement can be selected or varied via the adjustment device 374 (referenced generally), for example in configuring the apparatus 30 to accommodate a particular tubular graft structure (not shown) diameter. More particularly, and returning to FIG. 1A, in some embodiments, the adjustment device 374 includes a first disc body 400, a second disc body 402, and a control ring 404. In general terms, each of the disc bodies 400, 402 dictates a circular arrangement and radial spacing of the distal regions 100 relative to one another, with the control ring 404 adapted to effectuate a change in radial spacing when operated by a user.

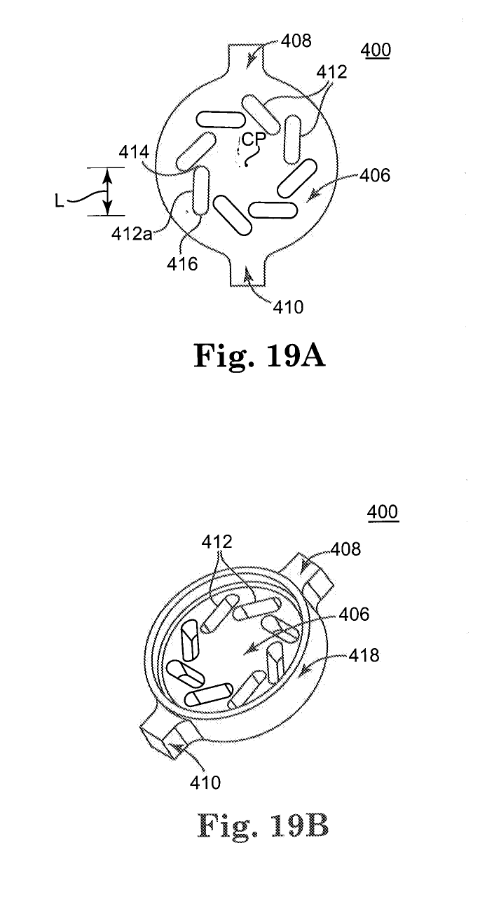

With reference to FIGS. 19A and 19B, the first disc body 400 can include a disc 406 and opposing tabs 408, 410. The disc 406 is sized to be rotatably mounted within the housing 370 (FIG. 1B), and forms a plurality of drive slots 412. Each of the drive slots 412 extends through a thickness of the disc 406, and is sized to slidably receive a respective one of the drive assemblies 32 (FIG. 1A). In particular, each of the drive slots 412 is defined by a width that approximates, or is slightly greater than, a diameter of the outer member 40 (FIG. 1) of a corresponding one of the drive assemblies 32, and a length L that is greater than a diameter of the outer members 40. While eight of the drive slots 412 are illustrated in FIGS. 19A and 19B, any other number, either greater or lesser, is also acceptable, so long as the number of drive slots 412 provided is not less than the number of delivery assemblies 32. Regardless, the drive slots 412 are equidistantly spaced about a center point CP of the disc 406, extending in an angular fashion relative to one another. More particularly, each of the drive slots 412 extends in a non-radial fashion relative to the center point CP. For example and with specific reference to FIG. 19A, the drive slot 412a includes a first end 414 and a second end 416, with the length L of the drive slot 412a being defined as a distance between the ends 414, 416. Relative to the center point CP of the disc 406, a radial distance between the first end 414 and the center point CP is less than a radial distance between the second end 416 and the center point CP. However, a difference between the second end 416 radial distance and the first end 414 radial distance is less than the slot length L (it being understood that were the drive slot 412a oriented in a purely radial manner relative to the center point CP, the difference in radial center point distance between the ends 414, 416 would be identical to the length L). As described below, this angular orientation of the drive slots 412 facilitates radial movement of the delivery assembly distal regions 100 with rotation of the first disc body 400.

To facilitate rotation of the disc 406 via the control ring 404, one or more of the tabs 408, 410 can be included. As shown in FIGS. 19A and 19B, the tabs 408, 410 extend from the disc 406, preferably at opposite sides thereof. Although the first disc body 400 is shown as including two of the tabs 408, 410, in other embodiments, a greater or lesser number can be provided. Regardless, the tabs 408, 410 are sized for assembly to the control ring 404 as described below.

Finally, the first disc body 400 can include an annular hub 418 extending distal the disc 406, as reflected in the rear perspective view of FIG. 19B. The annular hub 418 provides a bearing surface for interfacing with a corresponding surface 419 (FIG. 1A) of the housing 370 (FIG. 1A) in supporting the first disc body 400 upon final assembly. In other embodiments, the annular hub 418 can be eliminated.

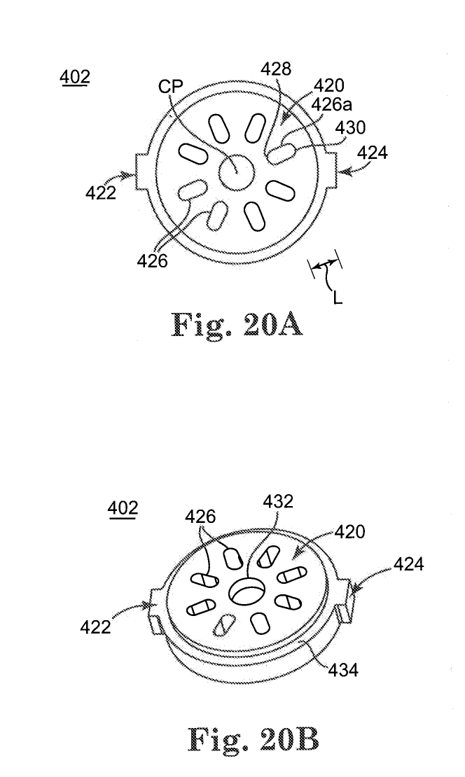

Returning to FIG. 1A, and with additional reference to FIGS. 20A and 20B, the second disc body 402 includes a disc 420 and tabs 422, 424. The disc 420 forms a plurality of guide slots 426. The guide slots 426 are equidistantly spaced relative to a center point CP of the disc 420, and are each sized to slidably receive a respective one of the delivery assemblies 32. For example, the guide slots 426 each have a width slightly greater than a diameter of a respective one of the outer members 40, and a length that is greater than the outer member diameter. As with the first disc body 400, while eight of the guide slots 426 are illustrated, any other number, either greater or lesser, is also acceptable, with the number of guide slots 426 corresponding with the number of drive slots 410 (FIG. 19A). Regardless, the guide slots 426 extend in a radial fashion relative to the center point CP of the disc 420. For example, the first guide slot 426a is defined by a first end 428 and a second end 430 that combine to define the length L. With these designations in mind, the radial orientation of the guide slot 426a relative to the center point CP dictates that the difference in radial distance between the first and second ends 428, 430 relative to the center point CP is the same as the slot length L. Alternatively, a wide variety of other configurations are also acceptable. In some embodiments, however, the drive slots 426 each taper in width from the first end 428 to the second end 430 to promote guiding of a corresponding delivery assembly 32 in a radially outward fashion.

The tabs 422, 424 radially extend from the disc 420, from opposite sides thereof in some embodiments. The tabs 422, 424 can assume a variety of forms, and are otherwise adapted to facilitate mounting of the second disc body 402 within the housing assembly 36 as described below. Along these same lines, the second disc body 402 can include other mounting features, such as a central aperture 432 and a shoulder 434 (as best shown in FIG. 20B). Alternatively, other mounting features can be provided, and the tabs 422, 424, the aperture 432 and/or the shoulder 434 can be eliminated.

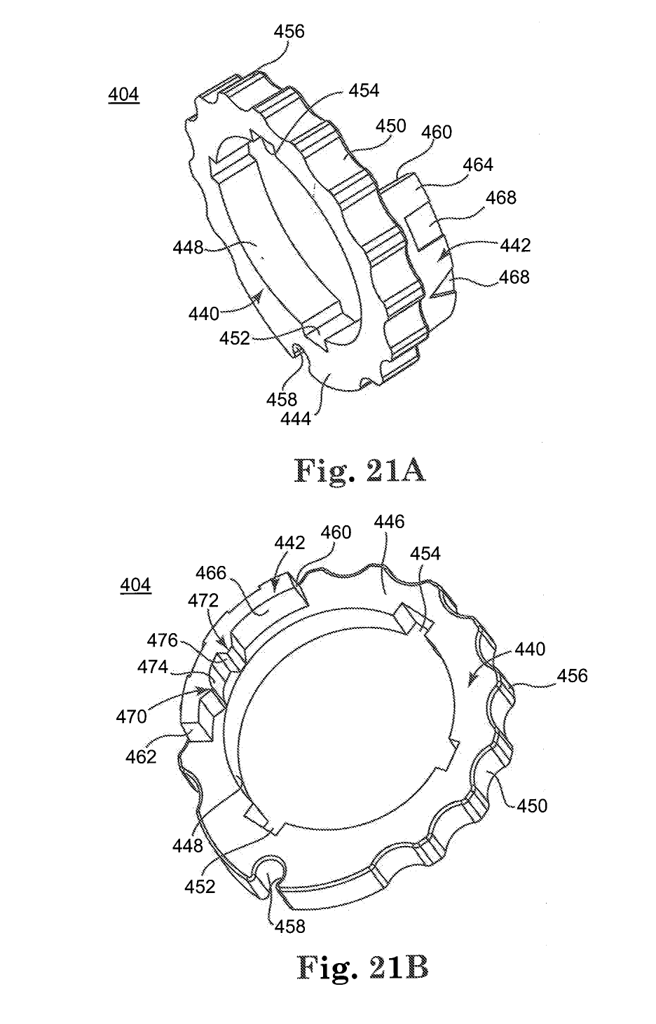

With reference to FIGS. 1A, 21A and 21B, the control ring 404 includes a ring body 440 and a shoulder 442. The ring body 440 is generally annular in shape and is defined by a leading face 444, a trailing face 446, an interior face 448, and an exterior face 450. The interior face 448 defines an inner diameter of the ring body 440 that is commensurate with a diameter of the disc 406 (FIG. 19A) of the first disc body 400 (FIG. 19A). Further, the ring body 440 forms first and second notches 452, 454 each sized to receive and retain a respective one of the tabs 408, 410 (FIG. 19A) of the first disc body 400. Thus, the first disc body 400 can be assembled within the ring body 440 via fixed engagement of the tabs 408, 410 within the notches 452, 454. Other forms of assembly are also acceptable, and in some embodiments, the control ring 404 and the first disc 406 can be integrally formed.

The exterior face 450 can include a contoured or scalloped surface portion 456 (referenced generally) otherwise configured to facilitate convenient interface by a user's fingers. Further, the ring body 440 can form a trough 458 along the exterior face 450 for selectively receiving a component of an optional transfer cap (not shown) as described below. Alternatively, the exterior face 450 can assume other forms that may or may not include the contoured portion 456 and/or the trough 458.

The shoulder 442 extends from the trailing face 446, and has a generally arcuate shape (in longitudinal extension) generally matching a curvature of the ring body 404. As shown, the shoulder 442 has a defined length (or arc length), extending between first and second ends 460, 462. In addition, the shoulder 442 has a top surface 464 (FIG. 21A) and a bottom surface 466 (FIG. 21B), and is positioned such that the top surface 464 is slightly recessed relative to the exterior face 450 of the ring body 440. In some embodiments, the top surface 464 can form or include nomenclature 468 that is otherwise indicative of a corresponding radial position or spacing of the drive assemblies 32 as dictated by a rotational position of the control ring 404 (described below). The bottom surface 466 forms or includes radially projecting teeth 470, 472. The teeth 470, 472 are adapted to interface with a corresponding component of the housing assembly 36 that otherwise serves to selectively lock the control ring 404 at a particular rotational position. For reasons made clear below, in some embodiments, the first tooth 470 defines a ramp face 474 in extension from the bottom surface 466, whereas the second tooth 472 provides a stop face 476. The ramp face 474 has a more tapered angle relative to the bottom surface 466 (e.g., greater than 100°), whereas the stop face 476 approximates a 90° angle in extension from the bottom surface 466. Other configurations are also acceptable.

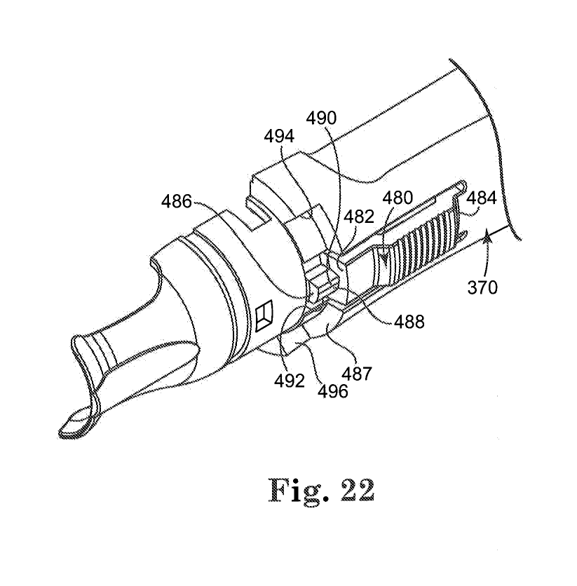

The housing 370 is configured to interface with the control ring 404, and in particular the shoulder 442. For example, as shown in FIG. 22, the housing 370 can include a locking finger 480 having a free end 482 and a hinged end 484. The hinged end 484 effectively serves as a living hinge, permitting inward deflection of free end 482 relative to a remainder of the housing 370 (with the locking finger 480 pivoting at the hinged end 484), while biasing the locking finger 480 to the position shown. The free end 482, in turn, includes a pawl 486. With combined reference to FIGS. 21B and 22, the pawl 486 is sized to interface with the teeth 470, 472 of the shoulder 442 (it being understood that though not illustrated, upon assembly of the control ring 404 to the housing 370, the control ring shoulder 442 is positioned within a recess 487 formed by the housing 370, with the ring body 440 rotatably positioned distal the recess 487). In particular, the pawl 486 can be selectively captured between the teeth 470, 472, thus “locking” the control ring 404 relative to the housing 370. Upon depression of the locking finger 480 (pivoting at the hinged end 484), the pawl 486 is displaced from between the teeth 470, 472, thus permitting rotation of the control ring 404. In some embodiments, a contact face 488 of the pawl 486 tapers from a first side 490 to a second side 492. This taper corresponds generally with the ramp face 474 of the first teeth 470 and the stop face 476 of the second rib 472, respectively. More particularly, the teeth 470, 472 and the pawl 486 are configured such that the pawl 486 more readily disengages from, and thus permits rotation of the shoulder 442 (and thus the control ring 404) “away” from, the first teeth 470, as compared to a more robust interface between the second teeth 472 and the pawl 486. Finally, the recess 487 is bordered by opposing end walls 494, 496 that serve to prevent overt rotation of the control ring 404 relative to the housing 370. In particular, rotation of the control ring 404 is prevented when one of the shoulder ends 460 or 462 contacts or abuts a corresponding one of the end walls 494, 496.

As best shown in FIG. 8, the first and second disc bodies 400, 402 are mounted to the housing 370 such that the second disc body 402 is distal the first disc body 400. In this regard, the second disc body 402 is affixed to the housing 370, and cannot rotate relative to the housing 370. Conversely, the first disc body 400 is rotatably mounted relative to the housing 370 via the control ring 404, with respective ones of the delivery assemblies 32 extending through corresponding ones of the drive slots 412 and the guide slots 426. The ring body 404 is rotatably assembled to the housing 370, and can be longitudinally captured relative to the housing 370 by a collar 500 (more completely illustrated in FIGS. 1A and 1B). Regardless, the exterior face 450 of the control ring 404 is exteriorly exposed relative to the housing 370, and thus is available for user interaction. The shoulder 442 is positioned within the recess 487 (FIG. 22) of the housing 370 as previously described.

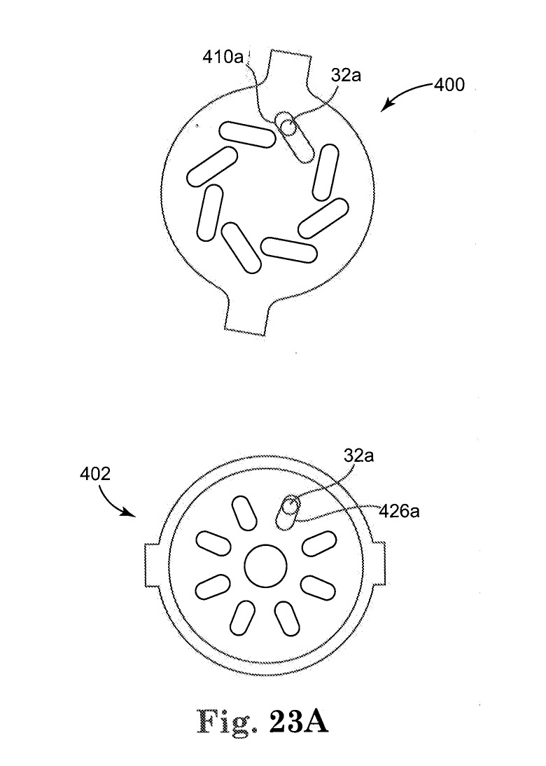

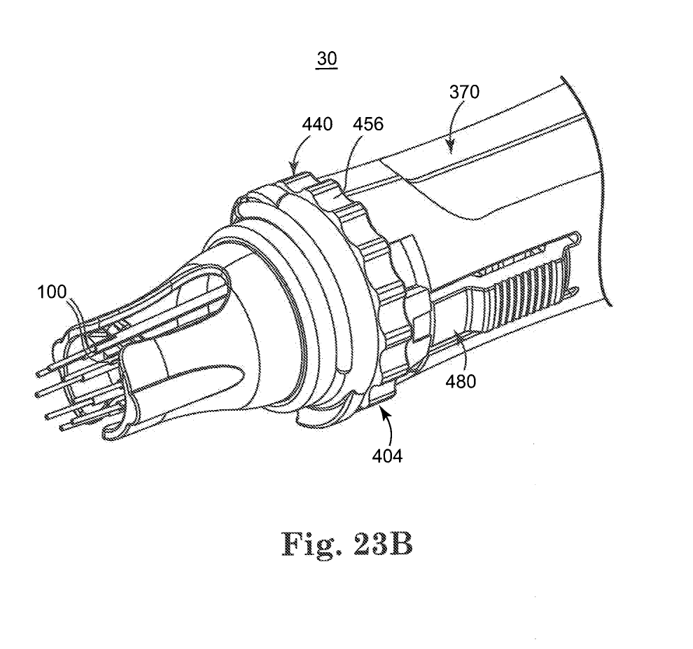

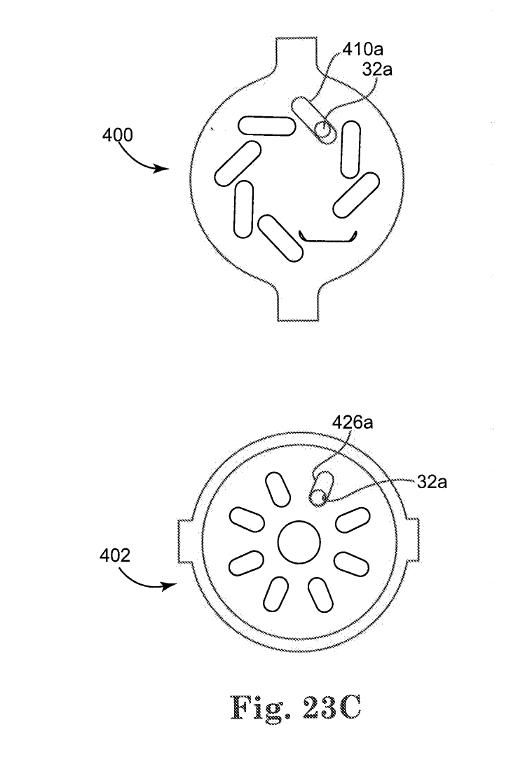

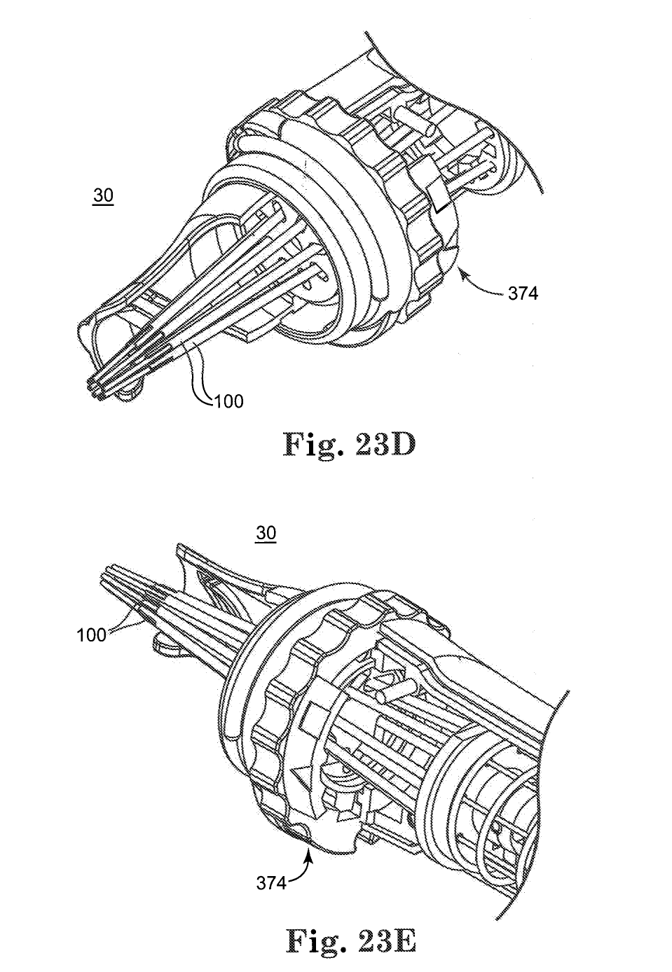

During use and returning to FIG. 17, the adjustment device 374 maintains the distal regions 100 of the delivery assemblies 32 in the generally circular arrangement shown, and can adjust a radial spacing of the distal regions 100 relative to one another (and thus of a diameter defined by the circular arrangement of the distal tips 76) as desired by a user. For example, in the arrangement of FIG. 17, a maximum radial spacing of the distal regions 100 is established by the adjustment device 374. As a point of reference, FIG. 23A schematically illustrates a position of one of the delivery assemblies 32a within or relative to a corresponding one of the drive slots 410a (of the first disc body 400) and a corresponding one of the guide slots 426a (of the second disc body 402).

To alter the radial spacing of the delivery assemblies 32 at the distal regions 100 thereof (and thus the diameter collectively defined by the circular arrangement of the distal tips 76), a user (not shown) actuates the first disc body 400. More particularly, and with specific reference to FIG. 23B, the user releases the control ring 404 relative to the housing 370 by depressing the locking finger 480 as previously described, and then applying a moment or rotational force onto the ring body 440 (e.g., at the scalloped surface 456), and thus the first disc body 400 (hidden in FIG. 23B). Notably, in some embodiments, a continual force on the locking finger 480 is required in order to rotate the control ring 404, such that changing a radial spacing of the delivery assembly distal regions 100 requires both of the user's hands (and is thus a concerted action by the user). Regardless and with additional reference to FIG. 8, the first disc body 400 rotates relative to the housing 370. With rotation of the first disc body 400, the delivery assemblies 32 are individually directed along the angularly oriented drive slots 412, thus applying a rotational moment force and radially inward force onto the drive assemblies 32. The second disc body 402, and in particular the guide slots 426 thereof, remains stationary such that the delivery assemblies 32 will not rotate distal the second disc body 402, but can and will move radially inwardly along a respective one of the guide slots 426. As a result, with rotational movement of first disc body 400, the distal region 100 of each of the delivery assemblies 32 deflects radially inwardly. This transitioned relationship of the first delivery assembly 32a relative to the slots 412a, 426a following rotation of the first disc body 400 is schematically illustrated in FIG. 23C. Following rotation of the first disc body 400, then, the adjustment device 374 transitions the distal regions 100 from the radial spacing/collective diameter of FIG. 17 to the reduced radial spacing/collective diameter reflected by the cutaway illustration of FIG. 23D (for purposes of comparison, a similar cutaway illustration is provided in FIG. 23E showing the distal regions 100 in the “open” or radially spaced position). Rotation of the first disc body 400 (FIG. 8) (e.g., via user force applied to the control ring 404) in an opposite direction increases the radial spacing/collective diameter of distal regions 100. Further, in some embodiments, the first disc body 400 can be selectively locked relative to the housing 370 so as to maintain the distal regions 100 at any desired radial spacing/collective diameter.

The adjustment device 374 as described above provides a marked improvement over available clip delivery-type anastomosis devices in which delivery assembly spacing is adjusted via a plunger that is moved axially relative to a distal end of the device's handle (as shown, for example, in U.S. Publication No. 2005/0070924). The rotational actuation facilitated by the adjustment device 374 presents an ergonomical improvement over previous designs. Alternatively, however, the adjustment device 374 can assume a wide variety of other configurations, and can instead, for example, incorporate axial actuation approach previously employed.

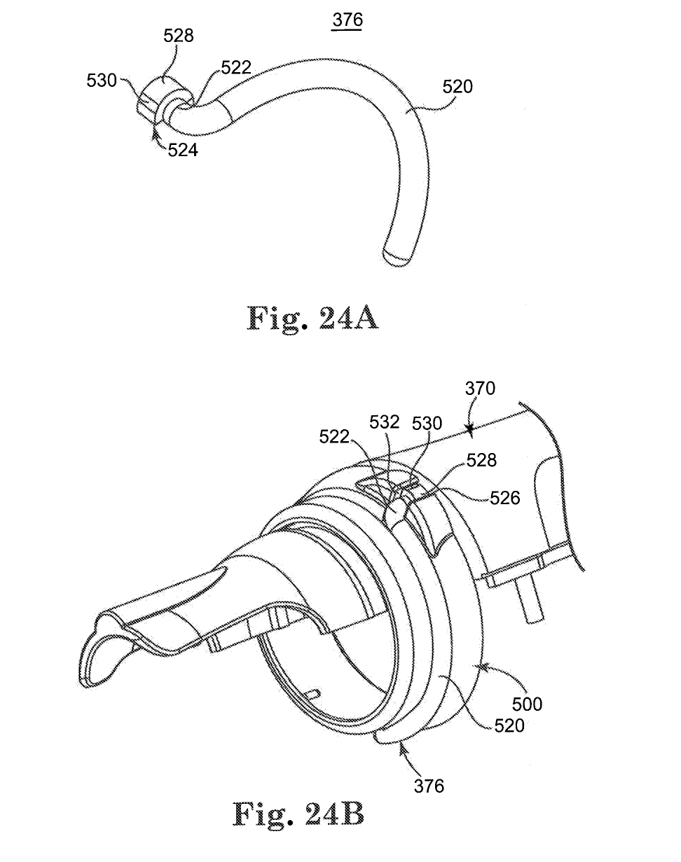

Returning to FIGS. 1A and 1B, the retainment clasp 376, where provided, can include a leading segment 520 and a trailing segment 522. With additional reference to FIG. 24A, the leading segment 520 has an arcuate shape, approximating a half circle. As a point of reference, FIG. 24B further illustrates the clasp 376 assembled to the collar 500 that in turn is assembled to the housing 370 (for ease of explanation, only a portion of the housing 370 is illustrated in FIG. 24B). With this in mind, a curvature of the leading segment 520 corresponds with a curvature of the collar 500. The trailing segment 522 is adapted for pivotable attachment to the collar 500 (or other component associated with or formed by the housing 370). For example, the trailing segment 522 can include or form a base 524 sized to be retained (e.g., frictional fit) within a slot 526 of the collar 500. The base 524 includes a curved surface 528 and a flat surface 530. With this arrangement, the base 524 can rotate relative to the slot 526 along the curved surface 528. However, contact between the flat surface 530 and the slot wall 532 impedes further rotation. Regardless, the clasp 376 is assembled such that the leading segment 520 can move or rotate in a radial fashion relative to the housing 370, and the clasp 376 will be held at any radial position/spacing relative to the housing 370 via connection of the trailing segment 522 to the collar 500 (or other component). With this configuration, the clasp 376 can readily be moved relative to the housing 370 to selectively retain (and subsequently release) a tubular graft structure (not shown) between the leading segment 520 and the housing 370. In other embodiments, however, the clasp 376 can be eliminated.

### Transfer Cap

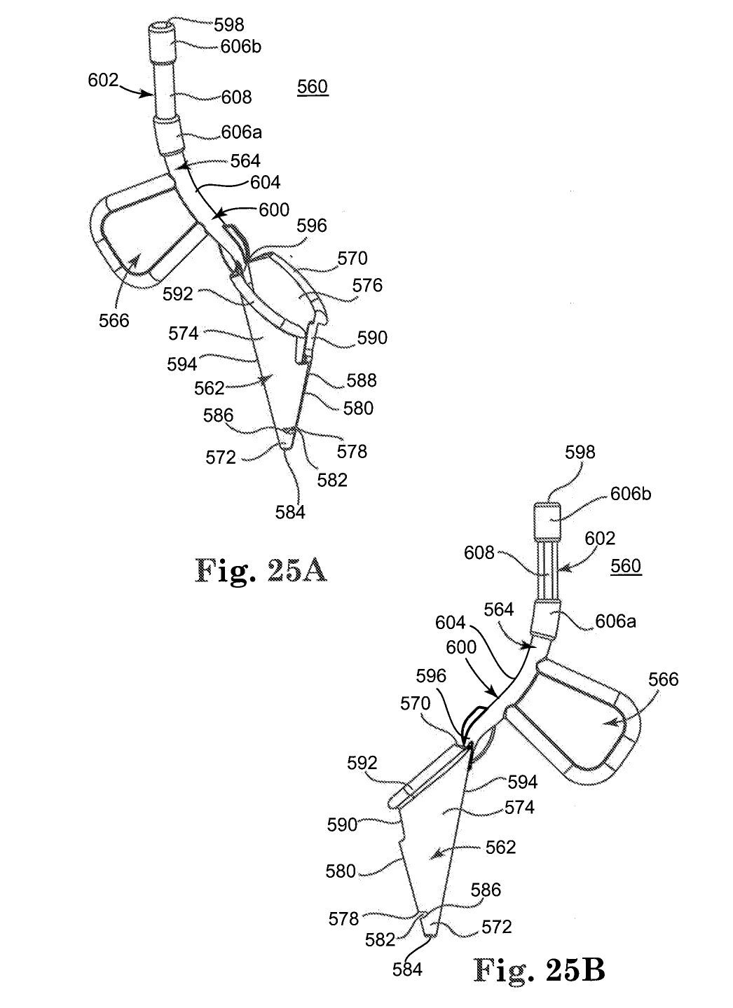

In some embodiments, the surgical connection apparatus 30 further includes or is used in conjunction with an optional transfer cap 560 as shown in FIGS. 25A and 25B. The transfer cap 560 includes a cone 562, a connector strip 564, and a handle 566. In general terms, the cone 562 is configured for releasable assembly to the nose 372 (FIG. 17) for encompassing the drive assembly distal regions 100 (FIG. 17). The strip 564 extends from the cone 562 and is adapted for releasable connection to a corresponding feature of the housing 370 (FIG. 17). Finally, the handle 566 extends from the strip 564 along a length thereof, and provides a surface for readily manipulating the protective cone device 560 by a user.

The cone 562 has a generally conical shape, tapering in diameter from a first end 570 to a closed tip 572. In this regard, a wall 574 of the cone 562 defines an enclosed region 576 (referenced generally in FIG. 25A). The wall 574 is only partially connected to the tip 572; the wall 574 terminates at a second end 578 that is otherwise spaced from the tip 572 relative to a first side 580 of the cone 562 (it being understood that while the cone 562 is generally circular in lateral cross-section, in the side plan view of FIG. 25B, the cone 562 can be defined as having “sides”). As such, a gap 582 is established between the second end 578 and the tip 572. With this configuration, the tip 572 provides a closed, smooth surface 584 for non-traumatically interfacing with bodily tissue. To enhance an overall flexibility of the tip 572 relative to the wall 574, in some embodiments one or more side cuts 586 are formed through the wall 574 that otherwise permit the tip 572 to deflect relative to the wall 574. This deflection characteristic better ensures that the tip 572 will not overtly “snag” against tissue as the tip 572 is dragged across the tissue (that might otherwise cause tissue damage). Along these same lines, the transfer cap 560 (or at least the cone 562) is preferably formed of a soft yet structurally tough material such as polyurethane that prevents the side cuts 586 from tearing or elongating as the tip 572 is deflected relative to the wall 574. That is to say, the transfer cap 560 material ensures that the tip 572 will not separate from the wall 574 during use.

In addition, the wall 574 includes a slit 588 along the first side 580 as shown in FIG. 25A. The slit 588 allows the wall 574 to be splayed apart from itself for reasons described below. Finally, an aperture 590 is defined along the first side 580, extending from the first end 570. The aperture 590 permits extension of a separate body (e.g., a tubular graft structure) “through” the cone 562.

The first end 570 forms a rim 592 that is otherwise characterized as having a radial projection relative to the wall 574. In this regard, the rim 592 is configured to nest within the channel 394 formed by the nose 372 as previously described with respect to FIG. 18. For example, the first end 570 can have an increasing height (relative to the tip 572) in extension from the first side 580 to a second side 594. This construction corresponds with the angled orientation of the channel 394, thus promoting assembly of the cone 562 to the nose 372 in only a single direction or orientation as described below.

The strip 564 extends from the first end 570 of the cone 562 at the second side 594. More particularly, the strip 564 includes a fixed end 596 and a free end 598. The fixed end 596 is connected to the cone 562, whereas the free end 598 is opposite the cone 562. With these conventions in mind, the strip 564 can pivot or deflect relative to the cone 562 at the fixed end 596/cone 562 interface. Further, the strip 564 is relatively rigid, and naturally assumes the curvilinear shape reflected in FIGS. 25A and 25B. For example, the strip 564 can be defined as including a trailing segment 600 and a leading segment 602. The trailing segment 600 extends from the fixed end 596 in a direction generally opposite or away from the second side 594 of the cone 562, and defines a bend or curve 604 projecting opposite the first end 570 of the cone 562. Relative to the orientation of FIG. 25B, then, the trailing segment 600 extends generally upwardly relative to the first end 570 of the cone 562. The leading segment 602 extends from the trailing segment 600 and terminates at the free end 598. The leading segment 602 includes first and second skirts 606a, 606b, and an engagement surface 608 between the skirts 606a, 606b. In this regard, the leading segment 602 can be generally circular in transverse cross-section, with the engagement surface 608 having a diameter less than a diameter of the skirts 606a, 606b. More particularly, a diameter of the engagement surface 608 is commensurate with a corresponding feature associated with the housing 370 (FIG. 1A) as described below. Finally, the leading segment 602 continues the generally upward (relative to the orientation of FIG. 25B) extension of the strip 564 relative to the cone 562 such that in a natural or undeflected state of the transfer cap 560, the free end 598 is positioned away from the first end 570 of the cone 562.

The handle 566 can assume a wide variety of forms, and in some embodiments extends from the trailing segment 600 of the strip 564. The handle 566 can extend from a side of the strip 564 generally opposite the cone 562 in providing a convenient surface for handling of the transfer cap 560 by a user. Other forms of the handle 566 are also acceptable, and in other embodiments, the handle 566 can be eliminated.

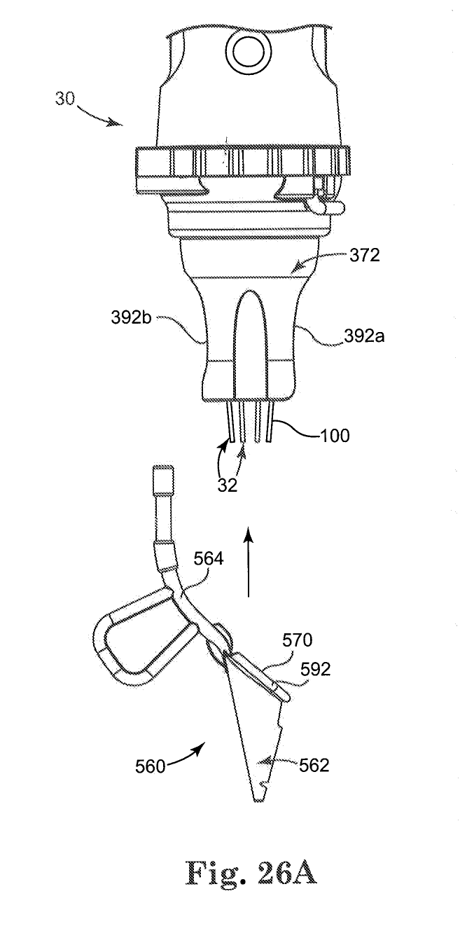

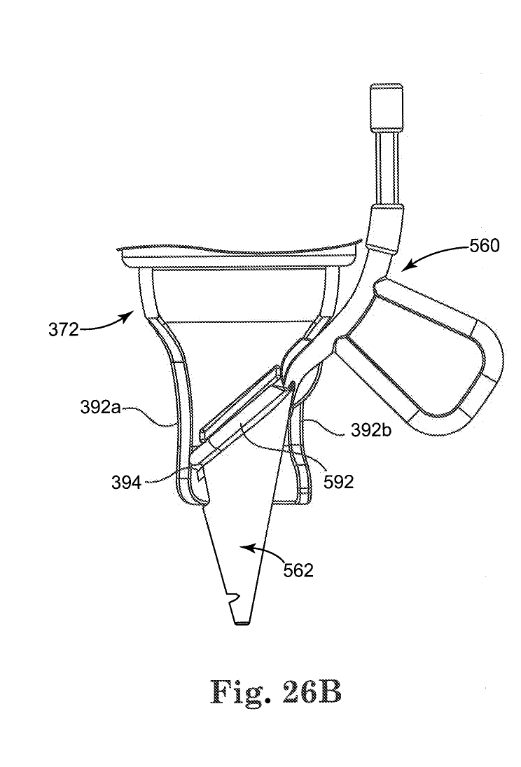

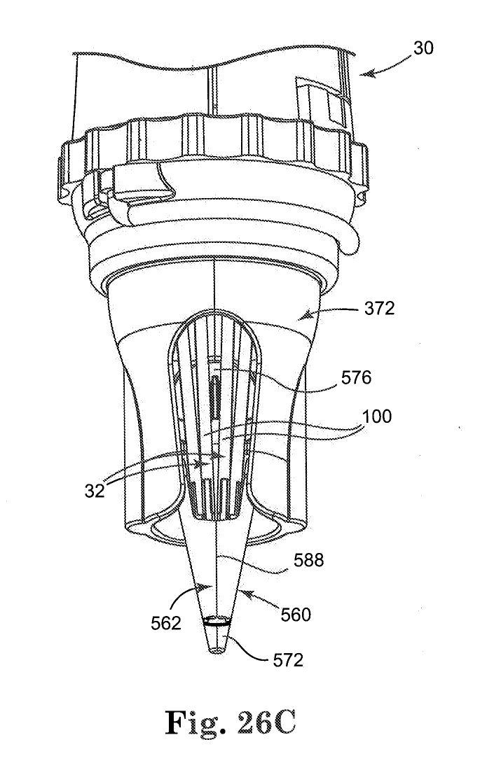

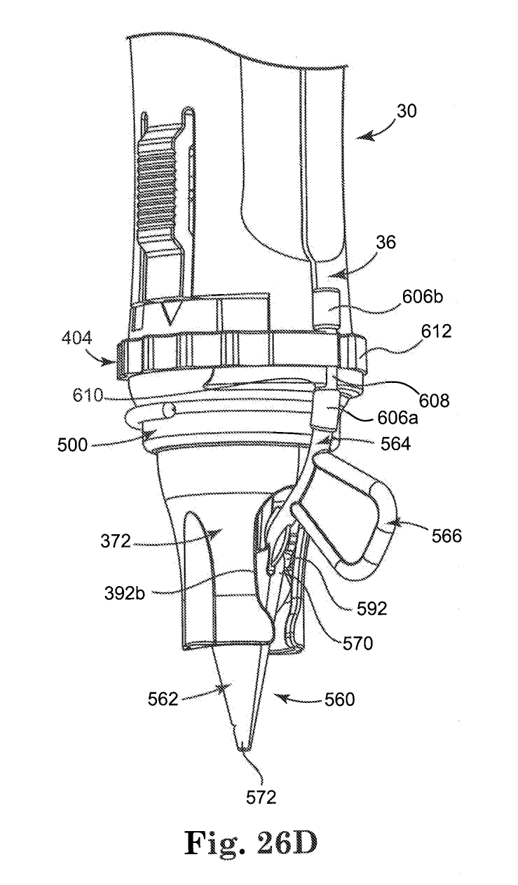

The transfer cap 560 can be assembled to, and removed from, a remainder of the surgical connection apparatus 30 as described below, beginning with FIG. 26A. The cone 562 is assembled to the nose 372 and about or around the distal regions 100 of the delivery assemblies 32 by axially aligning the cone 562 with the nose 372 distal the distal regions 100. The first end 570 is adjacent the nose 372, and the strip 564 is aligned with the second side opening 392b (referenced generally in FIG. 26A). The cone 562 is subsequently inserted into the nose 372 (in the direction of the arrow in FIG. 26A), with the cone wall 574 positioned about the distal regions 100. With insertion, the rim 592 moves into nested engagement with the channel 394 of the nose 372 as shown in FIG. 26B (it being noted that for ease of illustration, only the nose 372 and the transfer cap 560 are shown in FIG. 26B). Notably, the angled orientation of the channel 394 and the rim 592 dictates that proper insertion of the cone 562 occurs only with the strip 564 passing through the second side opening 392b (referenced generally). That is to say, were an attempt made to orient the cone 562 such that the strip 564 were aligned with the first side opening 392a of the nose 372, the rim 592 would not mate with the channel 394 as the cone 562 is slid into the nose 372. Once assembled within the nose 372, the distal regions 100 (FIG. 26A) are collectively encompassed or maintained within the enclosed region 576 of the cone 562, with the cone tip 572 positioned distal the distal regions 100 as shown in FIG. 26C.

Engagement of the transfer cap 560 to the housing assembly 36 is further enhanced by securing the strip 564 to the housing assembly 36 as shown in FIG. 26D that otherwise illustrates the strip 564 extending through the second side opening 392b. For example, and in some embodiments, the collar 500 of the housing assembly 36 includes or forms a slot 610 sized to frictionally receive and maintain the engagement surface 608 of the strip 564. In this regard, the strip 564 can readily be deflected relative to cone 562 in assembling the engagement surface 608 within the slot 610. Further, a longitudinal position of the collar 500, and thus of the slot 610, relative to the nose 372 corresponds with a length of the strip 564, and in particular a longitudinal distance between the engagement surface 608 at the first skirt 606a and the first end 570 of the cone 562. With these correlated dimensions in mind, then, when the rim 592 is nested within the channel 394 (FIG. 26B), the engagement surface 608 is readily directed into captured interface with the slot 610. While the slot 610 has been described as being provided by the collar 500, one or more other components of the housing assembly 36 can provide the desired point of interface with the strip 564. For example, the control ring 404 can also form an appropriately sized slot 612 for frictionally retaining the strip 564. Thus, when the control ring 404 is rotated such that the control ring slot 612 is aligned with the collar slot 610, the strip 564 can be retained in both slots 610, 612, with the second skirt 606b abutting against the control ring 404.

Upon final assembly and with reference to FIGS. 26C and 26D, the cone 562 surrounds and thus protects the distal regions 100 of the deliver assemblies 32, as well as any auxiliary body (not shown), for example a tubular graft structure, mounted thereto. Further, the cone tip 572 is distal the delivery assemblies 32, and thus provides a smooth contact surface during use. In addition, the transfer cap 560 facilitates controlled blood flow/hemostasis during an anastomosis procedure, as described below. When desired, the transfer cap 560 can be removed from the surgical connection apparatus 30 by a user simply grasping the handle 566 and applying a pulling force generally laterally away from the surgical connection apparatus 30. In response to this pulling force, the strip 564 is removed from the slot 610, and the cone wall 574 splays about the distal regions 100 (via the slit 588) such that the cone 562 can be removed from the nose 372 via the second side opening 392b.

Commensurate with the above description, the transfer cap 560 can assume a variety of other configurations. Further, the surgical connection apparatus 30 is highly useful apart from the transfer cap 560, such that in other embodiments, the transfer cap 560 is omitted.

Surgical Connection Apparatus 620

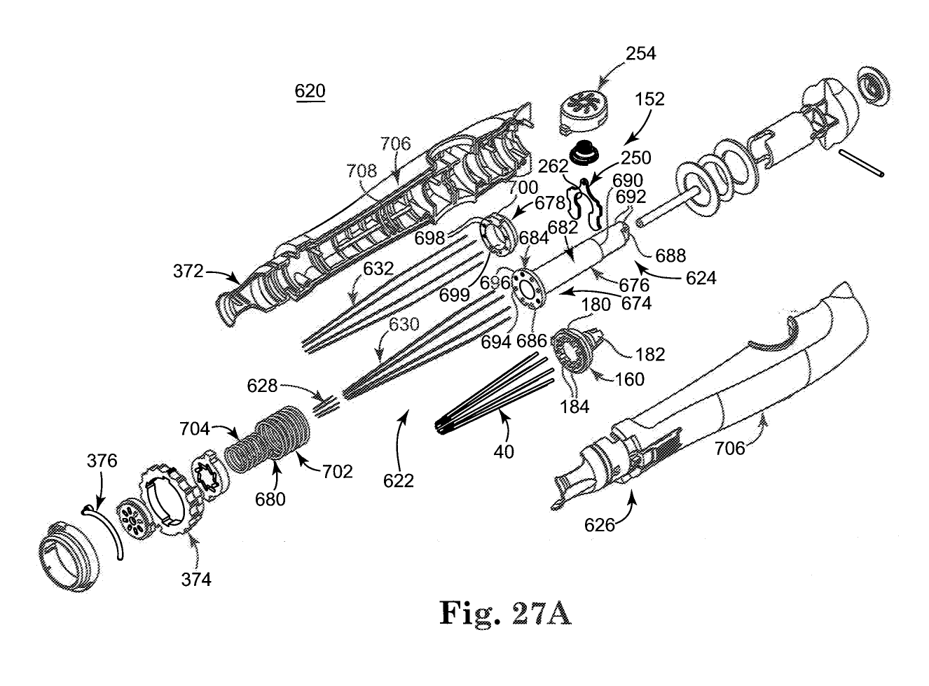

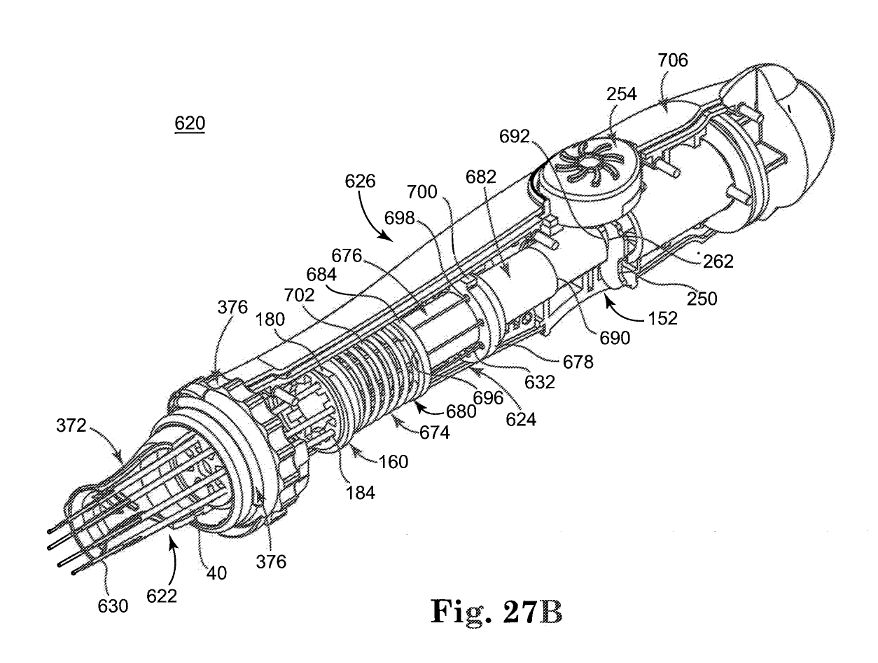

Another configuration of a surgical connection apparatus 620 in accordance with aspects of the present disclosure is shown in FIGS. 27A and 27B. The apparatus 620 is akin to the apparatus 30 (FIGS. 1A and 1B) previously described, with like components identified by like numbers. Thus, to the extent not otherwise specifically indicated, all of the above discussions relating to the apparatus 30, including components and operations thereof, apply equally to the embodiments associated with the surgical connection apparatus 620 described below.

The surgical connection apparatus 620 includes a plurality of drive assemblies 622, a drive mechanism 624, and a housing assembly 626 (each referenced generally in FIGS. 27A and 27B). In general terms, the housing assembly 626 maintains the delivery assemblies 622 and the drive mechanism 624. Each of the delivery assemblies 622 releasably maintains a self-closing clip 628 (as a point of reference, FIG. 27A illustrates a plurality of the clips 628, each in a deflected state). The drive mechanism 624, in turn, operates to effectuate substantially simultaneous release of the clips 628 from corresponding ones of the delivery assemblies 622. With this construction, then, a tubular graft structure (not shown) can be mounted to the delivery assemblies 622 and subsequently secured to a second structure (not shown) via deployment of the clips 628 in performing, for example, an anastomosis procedure.

Delivery Assembly 622

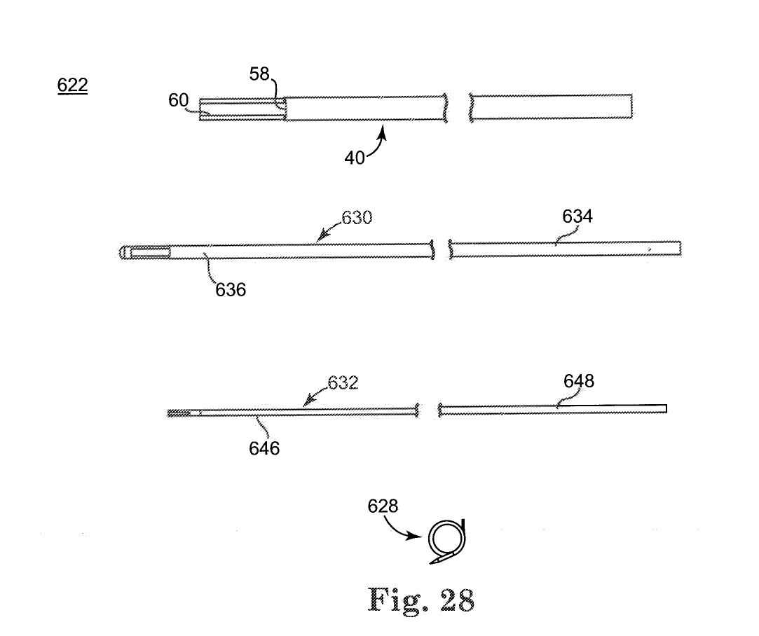

The surgical connection apparatus 620 can include any number of the delivery assemblies 622 (e.g., 4, 6, 8, etc.), and the delivery assemblies 622 are, in some embodiments, identical. Regardless, and with additional reference to the delivery assembly 622 shown in FIG. 28, the delivery assemblies 622 are akin to the delivery assemblies 32 (FIG. 2) previously described and include the outer member 40, an inner member 630, and a retention member 632. In general terms, the outer member 40 is sized to slidably receive the inner member 630. The inner member 630, in turn, is sized to slidably receive the retention member 632. Finally, the retention member 632 is configured to selectively maintain one of the clips 628. As described below, the members 40, 630, 632 combine to capture and retain one of the clips 628 in a deflected state (it being understood that in the view of FIG. 28, the clip 628 is in an undeflected or “relaxed” state). One or more of the members 40, 630, 632 can have a pre-formed, intermediate curve or bend to facilitate mounting to the housing assembly 626 (or can be forced to assume a curved or bent shape upon final assembly) as previously described.

The outer member 40 is, in some embodiments, identical to previous explanations provided with respect to FIGS. 3A and 3B. In general terms, the outer member 40 is a tubular body forming the lumen 58 and the aperture 60.

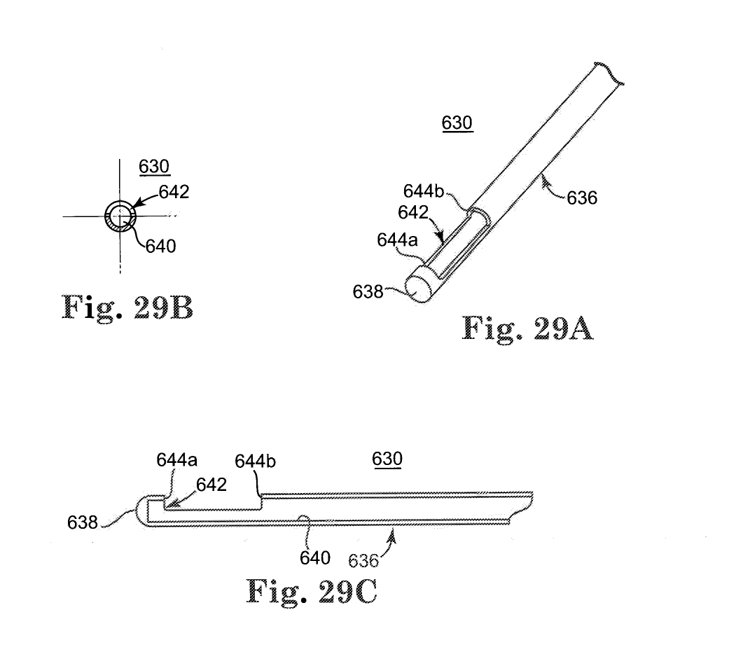

The inner member 630 is akin to the inner member 42 previously described with respect to FIGS. 4A-4C. As shown in FIGS. 29A-29C, however, certain variations can be provided. The inner member 630 is an elongated body defining a proximal segment 634 (FIG. 28) and a distal segment 636. The distal segment 636 terminates at a distal tip 638. The inner member 630 is at least partially tubular, and forms an internal passage 640. The internal passage 640 is sized to slidably receive at least a portion of the clip 628 (FIG. 28) in a deflected state, as well as one of the retention members 632 (FIG. 28). Further, the internal passage 640 is exteriorly open relative to the inner member 630 via a longitudinal window 642 that otherwise facilitates release of the clip 628 from the internal passage 640. The window 642 is defined by opposed, first and second ends 644a, 644b.

With the but one acceptable configuration of FIG. 29C, the internal passage 640 is longitudinally closed at the distal tip 638. The distal tip 638 can have the relatively curved exterior surface as shown, or can be configured to more readily pierce through a tissue structure (e.g., sharpened or needle-like). Regardless, the material(s), construction, and dimensions of the inner member 630 are, in some configurations, commensurate with those described above with respect to the inner member 42 (FIG. 4A-4C).

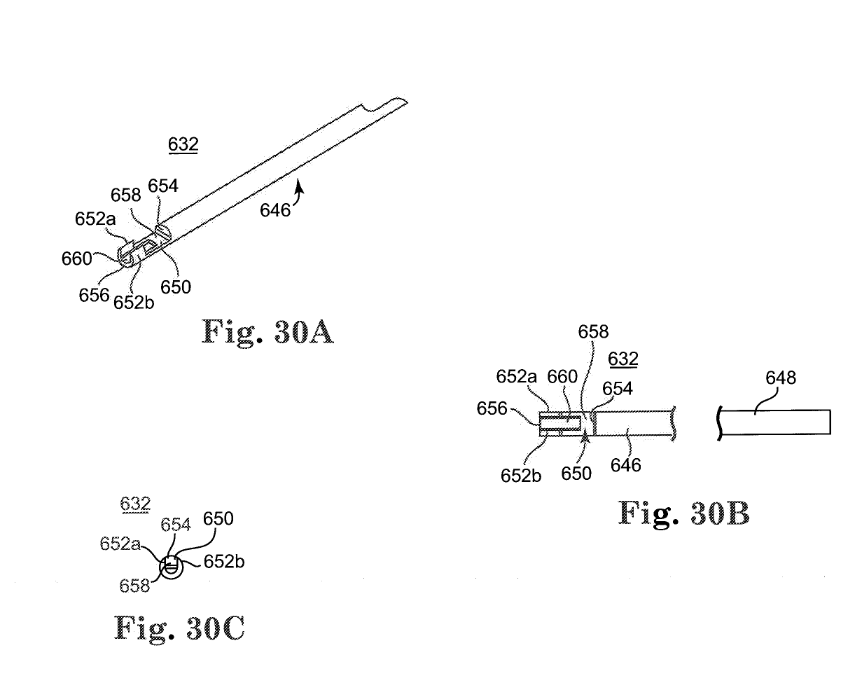

Returning to FIG. 28, the retention member 632 is an elongated body providing a distal section 646 and a proximal section 648. With additional reference to FIGS. 30A-30C, the retention member 632 can be a solid shaft, or, in other embodiments, is tubular in nature. Regardless, the distal section 646 is configured to releasably or partially engage with corresponding feature(s) of one of the clips 628. For example, the distal section 646 forms a slot 650 and opposing shoulders 652a, 652b. A wall 654 defines or forms a proximal end of the slot 650, with the shoulders 652a, 652b being formed opposite the wall 654 and extending to a distal end 656 of the retention member 632. The slot 650 is further defined or bounded by a platform surface 658, and the distal section 646 further forms a longitudinal channel 660 partially within, and extending distally from, the platform surface 658. The channel 660 continues to the distal end 656. With this construction, then, the shoulders 652a, 652b are laterally spaced from one another as shown.

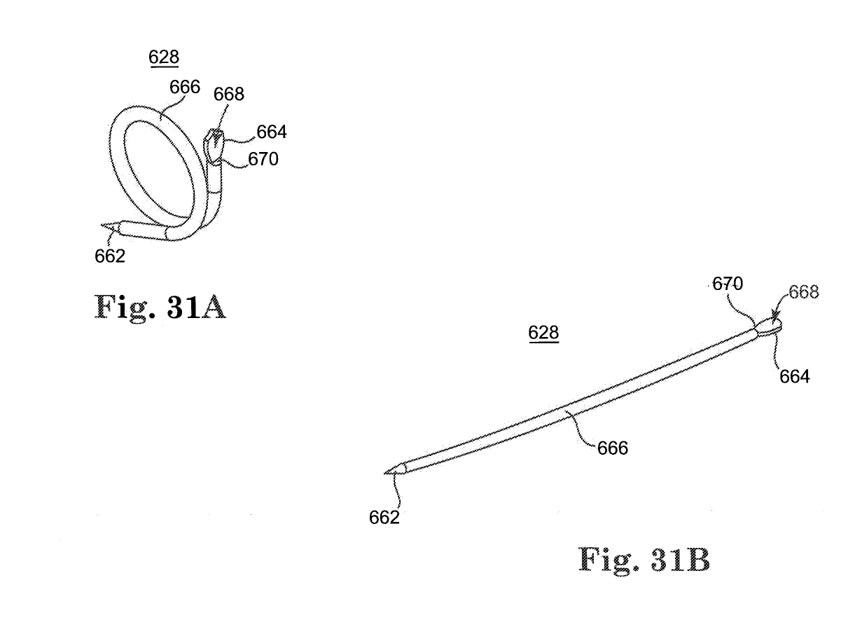

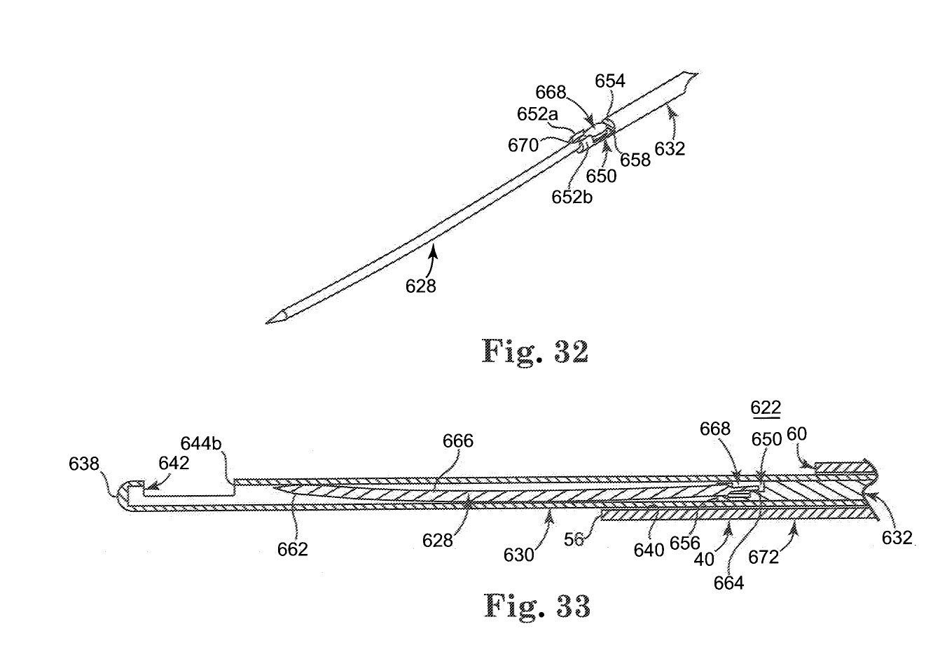

The above-described construction of the distal section 646 promotes selective engagement with the clip 628. As a point of reference, FIGS. 31A and 31B illustrate one embodiment of the clip 628 with which the retention member 632 is useful. As with the clip 44 (FIG. 2) previously described, the clip 628 defines opposing, first and second ends 662, 664. With the one construction of FIG. 31A, in an undeflected or “relaxed” state, a body 666 of the clip 628 naturally assumes a coil-like shape. In a deflected state (FIG. 31B), however, the clip 628 can be forced to a more linear shape. Upon removal of the force(s) otherwise causing deflection of the clip 628 to the deflected state, the clip 628 will self-revert to the undeflected state of FIG. 31A. With this in mind, the first end 662 can form a sharpened point. The second end 664 is configured to interface with the retention member 632. In particular, the second end 664 is flattened (as compared to a circular shape (in cross-section) of the body 666) to define a head 668. The head 668 has an enlarged outer dimension or diameter as compared to a remainder of the body 666. Accordingly, a neck 670 is defined immediately distal the head 668.

With the above construction, the clip 628 can be assembled to the retention member 632 as shown in FIG. 32. For purposes of ease of clarity, the clip 628 is shown in FIG. 32 in a deflected state, it being understood that in the absence of an external force, the clip 628 will not assume the relatively straight shape of FIG. 32. With additional reference to FIG. 30A, the head 668 is located within the slot 650 such that at least a portion of the head 668 rests against, or is supported by, the platform surface 658. The neck 670 extends between the shoulders 652a, 652b, nesting within the channel 660. Notably, a lateral spacing between the shoulder 652a, 652b approximates, or is slightly greater than, a diameter of the neck 670, such that the clip 628 is freely releasably from the retention member 632 via radial displacement through the spacing between the shoulders 652a, 652b. However, in the arrangement of FIG. 32, the head 668 is longitudinal captured relative to the retention member 632. More particularly, proximal movement of the clip 628 relative to the retention member 632 (or distal movement of the retention member 632 relative to the clip 628) is impeded via contact between the wall 654 and the head 668. Conversely, distal movement of the clip 628 relative to the retention member 632 (or proximal movement of the retention member 632 relative to the clip 628) is impeded via contact between the head 668 and the shoulders 652a, 652b.

With the above features of the retention member 632 in mind, a distal region 672 of the delivery assembly 622 in a pre-deployment or “retained” state is shown in FIG. 33, including the clip 628 retained thereby. The inner member 630 is slidably disposed within the outer member 40 such that the distal tip 638 of the inner member 630 extends distally beyond the distal end 56 of the outer member 40. In this regard, the outer and inner members 40, 630 are arranged relative to one another such that the window 642 of the inner member 630 “faces” the aperture 60 of the outer member 40. The retention member 632 is slidably disposed within the passage 640 of the inner member 630, with the distal end 656 of the retention member 632 being proximal the proximal end 644b of the window 642. Further, the clip 628 is coupled to the retention member 632 as previously described, with the head 668 being supported within the slot 650. The inner and retention members 630, 632 are arranged relative to one another such that the slot 650 of the retention member 632 “faces” the window 642 of the inner member 630. A longitudinal position of the retention member 632 relative to the inner member 630 in the pre-deployed state of FIG. 33 locates the first end 662 of the clip 628 proximal the proximal end 644b of the window 642. With this arrangement, then, the first end 662 of the clip 628 bears against an interior surface of the inner member 630, thereby capturing the clip 628 relative to the distal region 672 (i.e., an entirety of the clip 628 is within the inner member 630, proximal the window 642). The clip 628 is stressed to the deflected orientation of FIG. 33 such that the body 666 of the clip 628 bears against the inner member 630, forcing or biasing the ends 662, 664 to the positions shown in FIG. 33.

The clip 628 can be deployed or released from the delivery assembly 622 upon transitioning of the delivery assembly 622 from the retained state of FIG. 33. More particularly, with proximal movement of the inner member 630 relative to the retention member 632 and the outer member 40 (and/or distal movement of the retention member 632 relative to the inner member 630 and the outer member 40), the first end 662 of the clip 628 is located within the window 642. For example, as the inner member 630 is moved proximally over the retention member 632, the clip 628 remains stationary, with the inner member 630 sliding over the clip 628. Once the first end 662 is distal the proximal end 644b of the window 642, the first end 662 is no longer retained by the inner member 630. The shape memory characteristic of the clip 628 thus causes the “exposed” portion of the clip 628 to naturally revert back toward an undeflected or relaxed state such that the first end 662 naturally beings to deploy through the window 642. The second end 664 of the clip 628 remains captured by the distal region 672 with this initial deployment of the first end 662 due to continued engagement of the head 668 with the retention member 632 (and the inner member 630) as previously described. With continued proximal movement of the inner member 630 relative to the retention member 632, additional portions of the clip 628 continuously deploy through the window 642. Full release of the clip 628 occurs as the window 642 is radially aligned with the slot 650 of the retention member 632. At this orientation, the head 668 releases from the retention member 632 (again, due to the shape memory of the clip 628), passing through the window 642. With the one approach in which the inner member 630 is moved over the retention member 632 in a proximal fashion, the outer member 40 remains stationary (i.e., the inner member 630 is moved proximally relative to the outer member 40). At the location of the complete release of the clip 628 (i.e., the window 642 aligned with the head 668), the window 642 is “within” the aperture 60 of the outer member 40. Thus, the clip 628 freely releases through the aperture 60.

As with previous embodiments, the selective capture and release of the clip 628 relative to the delivery assembly 622 can be facilitated with a wide variety of other configurations of the distal region 672 and/or the clip 628. Thus, the construction of FIG. 33 is but one non-limiting example.

Drive Mechanism 624

Returning to FIGS. 27A and 27B, and with the above understanding of the drive assemblies 622 in mind, the drive mechanisms 624 can assume a variety of forms configured to facilitate substantially simultaneous release of the clips 628 from respective ones of the delivery assemblies 622 by effectuating simultaneous, sliding movement of the inner members 630. In some embodiments, the drive mechanism 624 is akin to the drive mechanism 34 (FIG. 8) previously described, and includes a drive assembly 674 and the locking assembly 152. As with the drive mechanism 34, the drive assembly 674 is adapted to move the inner members 630 relative to the corresponding outer members 40 and the corresponding retention members 632 in a sliding fashion to effectuate release of the retained clips 628. The locking assembly 152, in turn, is adapted to retain the drive assembly 674 in a desired state prior to use, as well as to initiate operation of the drive assembly 674 in response to user actuation. While the following description relates to a deployment approach in which the inner members 630 are moved (proximally) by operation of the drive mechanism 624, in other embodiments, the drive mechanism 624 is configured to cause movement of the retention members 632 in deploying the clips 628.

The drive assembly 674 includes the first holder body 160, a second holder body 676, a third holder body 678, and a biasing assembly 680. In general terms, the first holder body 160 retains the outer members 40; the second holder body 676 retains the inner member 630; and the third holder body 678 retains the retention member 632. The biasing assembly 680 biases the second holder body 676 relative to the first and third holder bodies 160, 678. With this construction, then, movement of the second holder body 676 via operation of the biasing assembly 680 effectuates movement of the inner member 630 relative to the outer members 40 and the retention member 632.

As reflected in FIGS. 27A and 27B, the first holder body 160 can be identical in form to the descriptions provided above with respect to FIGS. 8, 9A, and 9B. In general terms, the first holder body 160 includes the base 180 forming the plurality of holes 184 sized for affixment to respective ones of the outer members 40, as well as slidable movement of the inner members 630 (and thus of the retention members 632 otherwise coaxially received within respective ones of the inner members 630). While FIG. 27A reflects the first holder body 160 as also including the neck 182, the neck 182 (and corresponding features) can be eliminated. More particularly, in contrast to the drive mechanism 34 (FIG. 8) described above, the biasing assembly 680 does not directly interface with the neck 182 (and related features) such that the neck 182 is not necessary.

The second holder body 676 is akin to the second holder body 162 (FIGS. 8 and 10A-10C) previously described, and is adapted to maintain, and dictate movement of, the inner members 630 as well as to interface with the biasing assembly 680 as described below. With this in mind, in some embodiments, the second holder body 676 includes a central shaft 682 and a flange 684. The central shaft 682 defines a leading end 686 and a trailing end 688. The flange 684 is disposed at the leading end 686. An intermediate step 690 is formed along the central shaft 682 at which a diameter of the shaft 682 is reduced (i.e., an outer diameter of the shaft 682 at the leading end 686 is greater than an outer diameter at the trailing end 688). Finally, the shaft 682 forms opposing notches 692 at the trailing end 688.

The flange 684 extends radially outwardly from the shaft 682. An outer diameter defined by the flange 684 is generally sized in accordance with the biasing assembly 680 as described below. Further, the flange 684 forms bores 694 sized to receive respective ones of the inner members 630, as well as to permit sliding movement of respective ones of the retention member 632. Regardless, the flange 684 forms a trailing face 696 of the second holder body 676.

The third holder body 678 is ring-shaped, forming a plurality of holes 698 each sized to receive a respective one of the retention members 632. Further, the third holder body 678 defines a central aperture 699 sized to slidably receive the shaft 682 of the second holder body 676. As described below, upon final assembly, the third holder body 678 is spatially affixed relative to the housing assembly 628, and thus can include a radial tab 700 that facilitates this construction. Alternatively, the third holder body 678 can assume a variety of other forms.

The biasing assembly 680 can also assume a variety of forms, and in some embodiments includes first and second springs 702, 704. As shown in FIG. 27A, the springs 702, 704 are compression springs, with the first spring 702 having a diameter greater than that of the second spring 704. More particularly, diameters of the springs 702, 704 are selected such that the first spring 702 is disposed about an outer diameter collectively defined by the inner members 630, whereas the second spring 704 is disposed within a diameter collectively defined by the inner members 630. With this construction, then, the spring 702, 704 combine to provide a relatively large spring force. Other configurations can also be employed for the biasing assembly 680. For example, the biasing assembly 680 can consist of a single spring.

FIG. 27B illustrates the drive assembly 674 upon final assembly and in a “pre-firing” state. The shaft 682 of the second holder body 676 is coaxially received within the central aperture 699 of the third holder body 678, with the third holder body 678 being spatially affixed relative to the housing assembly 626. The first holder body 160 is assembled to the housing assembly 626 at a location distal the flange 684 of the second holder body 676. In this regard, the holder bodies 160, 676, 678 are arranged such that respective ones of the retention member 632 retained by the third holder body 678 slidably extend within respective ones of the inner member 630 otherwise retained by the second holder body 676. Further, respective ones of the inner members 630 slidably extend within respective ones of the outer members 40 otherwise retained by the first holder body 160. The biasing assembly 680 is disposed between the first and second holder bodies 160, 676. In particular, each of the springs 702, 704 are compressed between the base 180 of the first holder body 160 and the trailing face 696 of the flange 684 of the second holder body 676. As reflected in FIG. 27B, the first spring 702 exteriorly surrounds the inner member 630, whereas the second spring 704 is disposed within the diameter effectively defined by the inner member 630.

The locking assembly 152 can assume the configurations previously described. Thus, in the pre-firing state of FIG. 27B, the plunger body 250 engages the central shaft 682 within the notches 692. Upon depression of the actuator 254, the plunger body 250 moves downwardly (relative to the orientation of FIG. 27B), aligning the recess 262 with the shaft 682. The spring force exerted by the biasing assembly 680 then causes the second holder body 676 to slide proximally, with the shaft 682 sliding within the third holder body 678 as well as within the recess 262 of the plunger body 250. Proximal movement of the second holder body 676 continues until the plunger body 250 contacts the intermediate step 690. With proximal movement of the second holder body 676, the retained inner members 630 proximally move relative to the outer members 40 and the retention members 632, respectively. Alternatively, a wide variety of other configurations for the drive mechanism 624 can be employed that provide simultaneous proximal movement of the inner members 630 relative to the retention members 632 and/or distal movement of the retention members 632 relative to the inner members 630 upon actuation by a user.

### Housing Assembly 626

Returning to FIGS. 27A and 27B, the housing assembly 626 is, in some embodiments, akin to the housing assembly 36 (FIGS. 1A and 1B) previously described. In this regard, the housing assembly 626 maintains the delivery assemblies 622 and the drive mechanism 624, and includes the housing 706, the nose 372, the optional adjustment device 374, and the optional retainment clasp 376. As with previous embodiments, the housing 376 has features adapted to maintain the drive mechanism 624, portions of the delivery assemblies 622, the adjustment device 374, and the clasp 376. The nose 372 extends from the housing 706, and provides a guide surface for positioning of the apparatus 620 during an anastomosis or other procedure. The adjustment device 374 is connected to the delivery assemblies 622 and facilitates arrangement of the delivery assemblies 622 at a desired radial spacing as previously described. Finally, commensurate with previous descriptions, the clasp 376 is pivotably secured to the housing 706 and facilitates selective securement of the graft structure (not shown) (e.g., a vein) to the housing 706.

The housing 706 is akin to the housing 370 (FIG. 8), and can assume a variety of shapes and sizes conducive for convenient handling by a user. Any of the features previously described with respect to the housing 370 can be incorporated with the housing 706. In addition, and as best shown in FIG. 27A, the housing 706 forms an annular slot 708 adapted to capture the third holder body 678 in a non-movable fashion. Thus, and as shown in FIG. 27B, the third holder body 678 is held within the slot 708, and is thus spatially affixed relative to the housing 706. Conversely, the second holder body 676 is slidable within the housing 706 (e.g., within the third holder body 678) as previously described.

Other features of the housing assembly 626, including the nose 372, the optional adjustment device 374, and the optional retainment clasp 376 can assume any of the forms described above. That is to say, the housing 706 is configured in manners similar to those described with respect to the housing 370 (FIG. 8) to facilitate assembly and, where appropriate, operation of the nose 372, the adjustment device 374, and the retainment clasp 376 as set forth elsewhere in this specification.

### Transfer Cap

Though not shown in FIGS. 27A and 27B, in some configurations, the surgical connection apparatus 620 further includes or is used in conjunction with the optional transfer cap 560 (FIGS. 25A and 25B) previously described. Thus, all previous descriptions of the transfer cap 560 relative to the apparatus 30 (FIG. 1A) are equally applicable to the surgical connection apparatus 620 of FIGS. 27A and 27B.

### Methods of Use

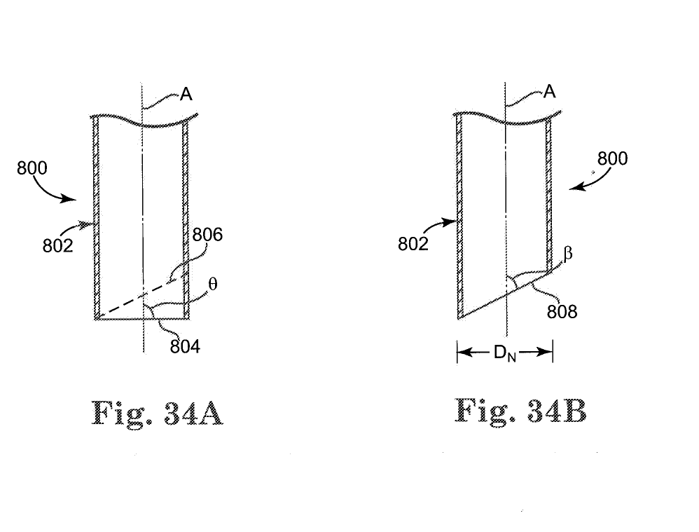

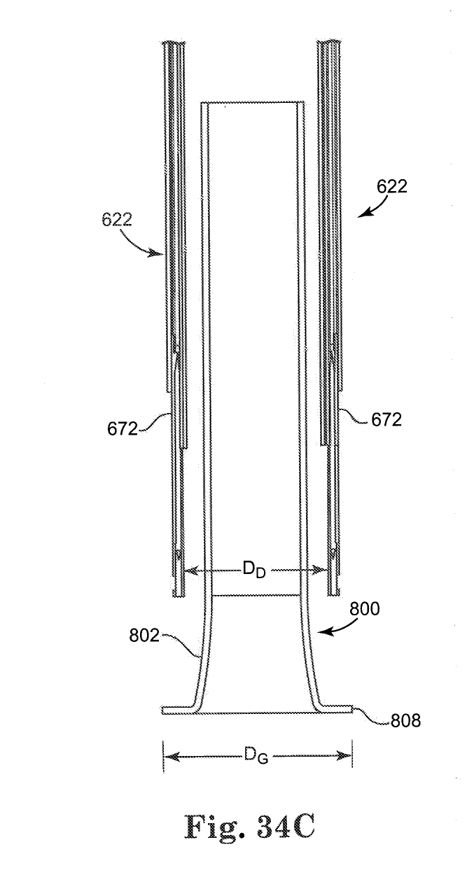

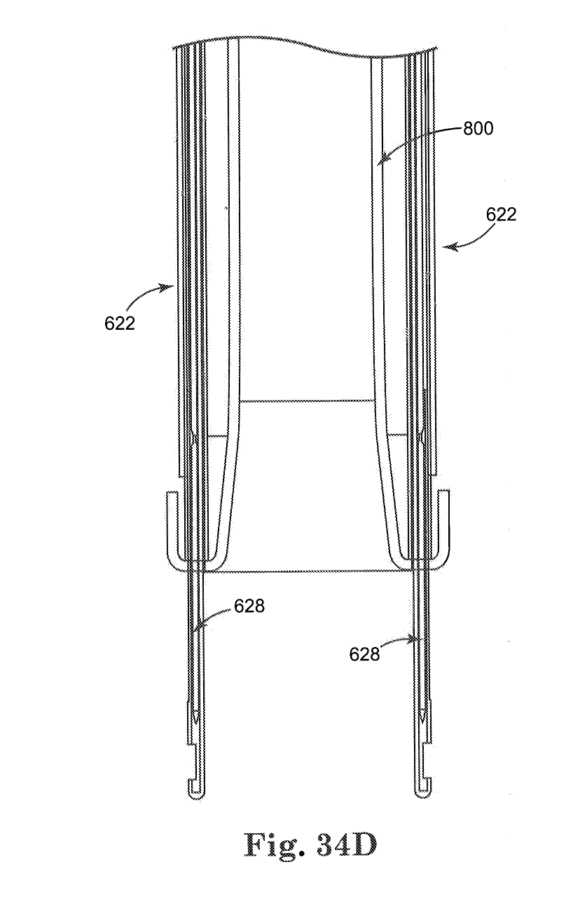

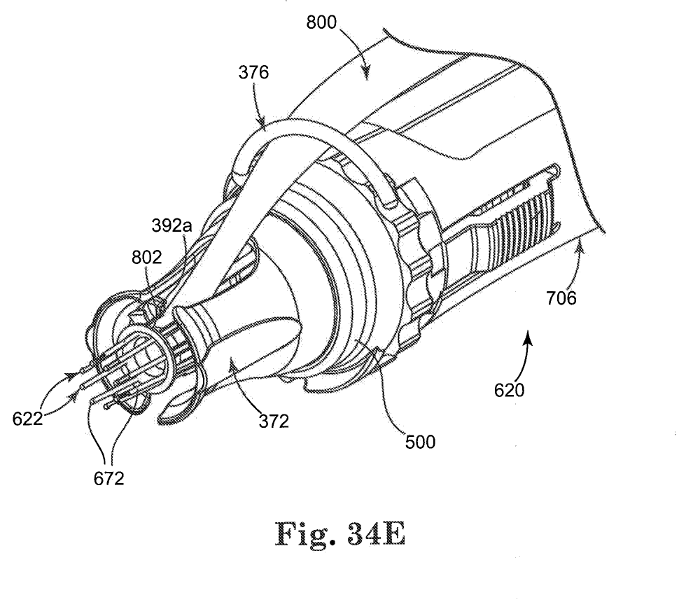

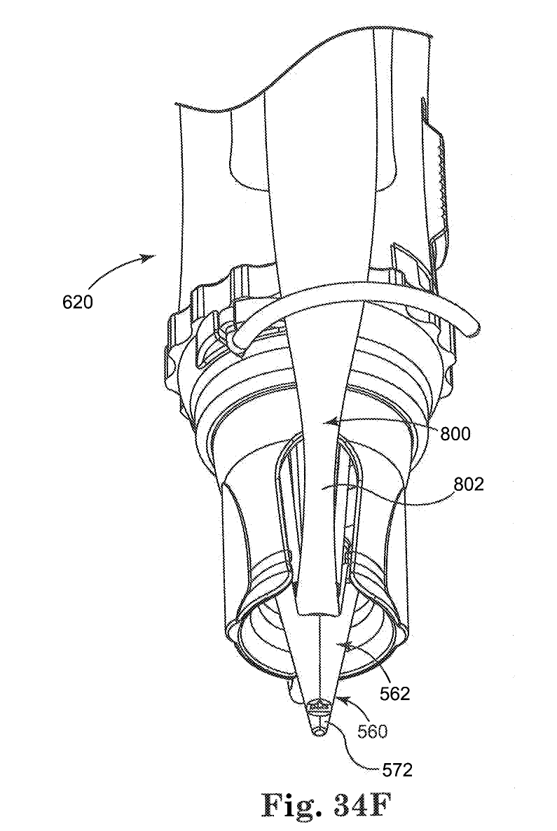

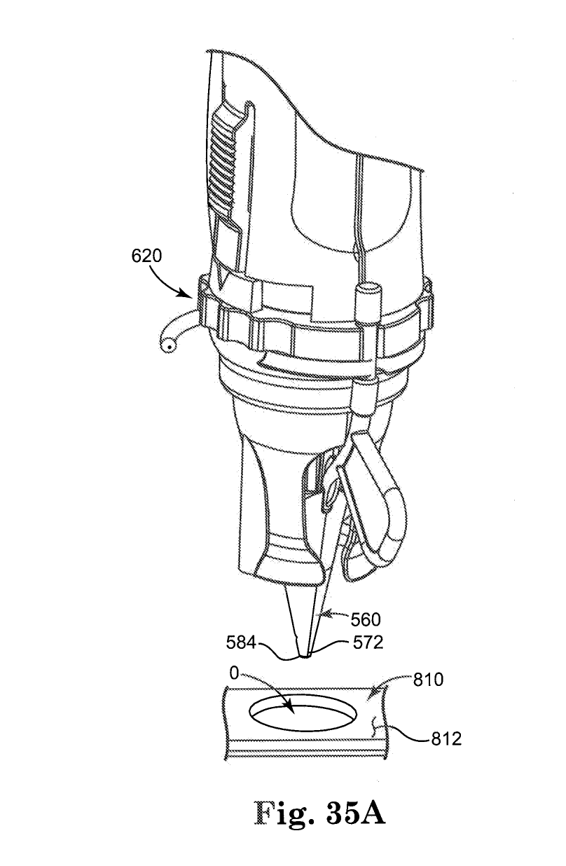

The surgical connection apparatuses 30, 620 described above can be used to, for example, perform an anastomosis procedure. While the following description makes reference to use of the surgical connection apparatus 620, the surgical connection apparatus 30 is also useful in a similar manner. During use, a tubular graft structure is initially mounted to the surgical connection apparatus 620, and in particular the delivery assemblies 622. As a point of reference, FIG. 34A schematically illustrates a tubular graft structure 800, and in particular a distal portion 802 thereof. In some embodiments, prior to mounting to the surgical connection apparatus 620 (FIG. 27A), the tubular graft structure 800 can be cut at a desired beveled angle otherwise appropriate for a particular surgical application. To clarify, as initially provided to the surgeon, the distal portion 802 terminates at a distal end 804 that is substantially perpendicular relative to a longitudinal axis A of the distal portion 802 (represented by the angle Θ in FIG. 34A). In fact, operation of many available automated anastomosis connection tools essentially requires this perpendicular or “right angle” relationship of the distal end 804 relative to the longitudinal axis A in order to properly mount or “load” the graft structure onto the tool. While viable, this conventional right angle approach dictates that following the anastomosis procedure, the tubular graft structure 800 will extend at a generally right angle (i.e., perpendicular) from the tissue surface to which the tubular graft structure 800 is secured. This, in turn, may be less than optimal. The surgical connection apparatuses and related methods of use of the present disclosure overcomes this potential draw back by allowing mounting of a tubular graft structure the distal end of which is cut to a desired angle that is not otherwise perpendicular to the graft structure longitudinal axis.

Given the above, in some embodiments, the tubular graft structure 800 is cut at a desired angle or bevel prior to mounting to the surgical connection apparatus 620 (FIG. 27A). For example, a cut (identified by a dashed line 806) is imparted through the distal portion 802, resulting in a new or cut distal end 808 as shown in FIG. 27B. The cut distal end 808 forms or extends at an angle β relative to the longitudinal axis A that is less than 90° (e.g., in the range of 30°-70°); in other words, the cut distal end 808 represents a bevel cut, and is distinguishable from the right angle Θ (FIG. 34A) employed or required by previous tools. Alternatively, however, the surgical connection apparatus 620 of the present disclosure is equally useful with a tubular graft structure having a right angle distal end (e.g., the configuration of FIG. 34A) such that the following description is in no way limited to the bevel cut distal end 808.

Regardless of the exact form of the distal end 804 or 808, the distal portion 802 defines a normal graft diameter D_{N}. With these designations in mind, FIG. 34C illustrates the tubular graft structure 800 relative to the distal region 672 of several of the delivery assemblies 622 just prior to mounting. As shown, the distal portion 802 has been distended so as to define an increased or distended graft diameter D_{G} at the distal end 808 (with the distended diameter D_{G} being greater than the natural diameter D_{N}). Conversely, a diameter D_{D} defined by the delivery assembly distal regions 672 is less than the distended graft diameter D_{G} such that the distal regions 672 pierce through a thickness of the tubular graft structure 800 with movement of the delivery assemblies 622 and the tubular graft structure 800 relative to one another from the orientation of FIG. 34C.

FIGS. 34D and 34E illustrate the tubular graft structure 800 mounted to the delivery assemblies 622. As a point of reference, the clips 628 remain fully captured within the corresponding delivery assemblies 622 during and following initial mounting of the tubular graft structure 800. Further, FIG. 34E reflects that in some embodiments, the tubular graft structure 800 extends through the first side opening 392a of the nose 372, and can subsequently be secured to the housing 706 via the clasp 376 (e.g., the clasp 376 can be rotated as previously described, with the tubular graft structure being lightly captured between the clasp 370 and the collar 500). Where provided, the transfer cap 560 can then be assembled as part of the surgical connection device 620 as previously described and generally shown in FIG. 34F. The distal regions 672 (FIG. 34E) of the delivery assemblies 622 (FIG. 34E), as well as the distal portion 802 of the tubular graft structure 800, are encompassed within, and protected by, the cone 562, with the cone tip 572 providing a distal-most contact surface, and facilitating hemostasis as described below.

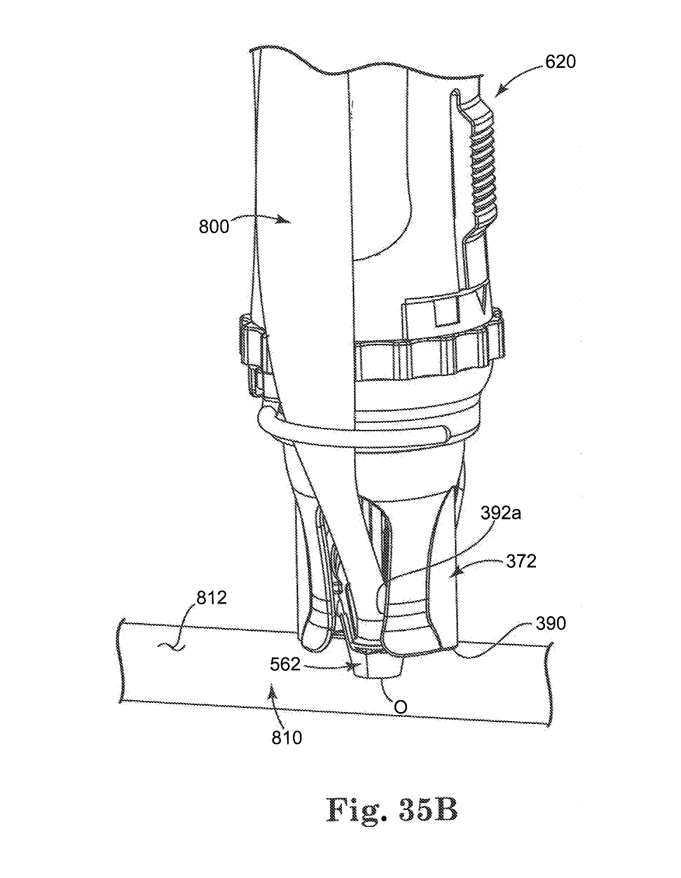

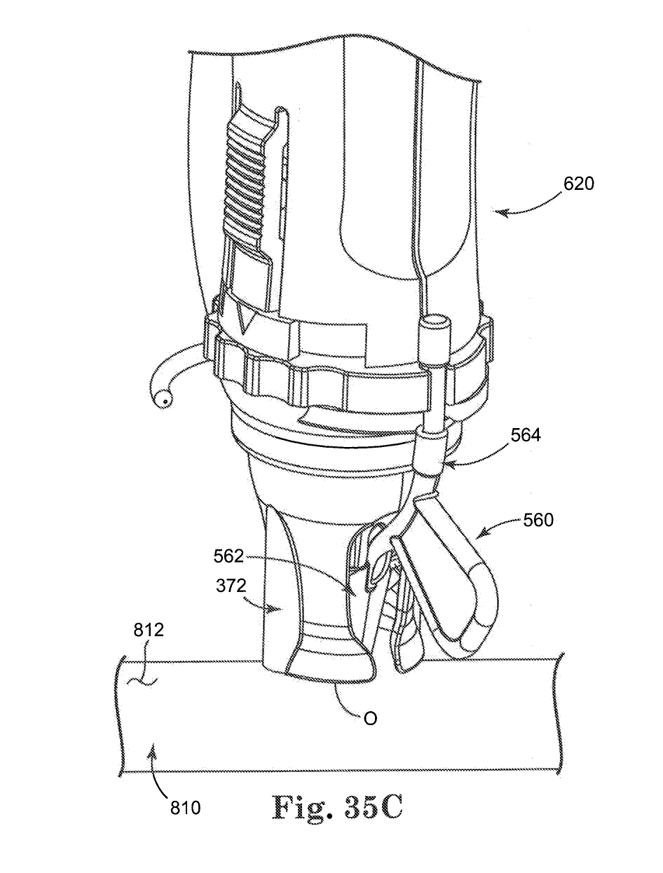

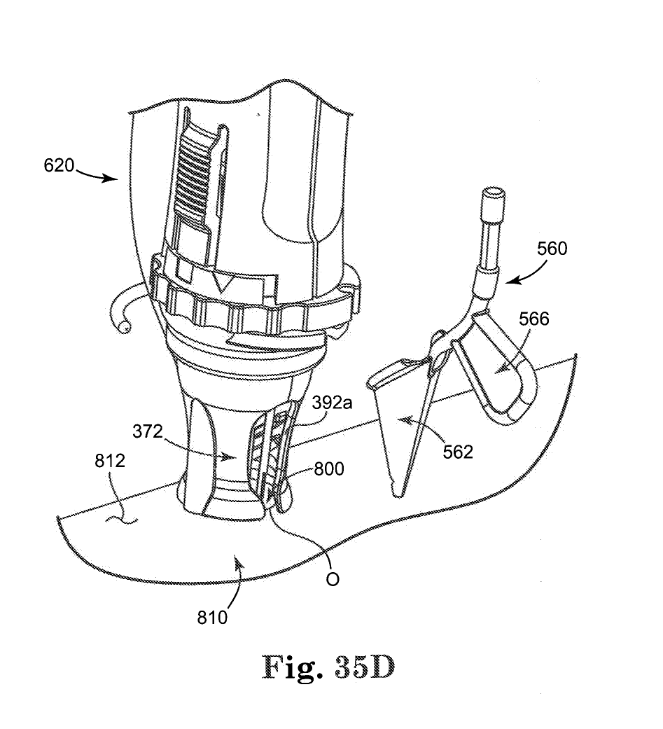

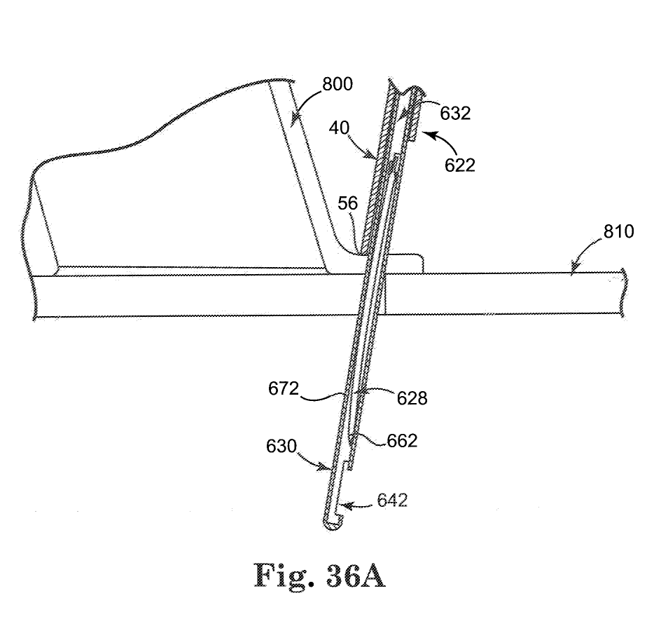

As shown in FIG. 35A, the anastomosis procedure then entails the surgeon forming an opening O in a second target structure 810 (e.g., an aorta) using, for example, a scalpel and/or an appropriate cutting device such as an aortic punch (not shown). When the opening O (e.g., aortotomy) has been completed, the surgeon removes the cutter or punch, and introduces the surgical connection apparatus 620. To this end, the surgeon's finger (not shown) can be immediately placed over the opening O (i.e., at an exterior surface 812 of the second structure 810) to impede flow of liquid (i.e., blood) from the confines of the second structure 810. The cone 562 and continued distal regions 672 (FIG. 34D) are inserted into the opening O. In instances where a diameter of the cone 562 (or, where the transfer cap 560 is not employed, an effective diameter of the delivery assembly distal regions 672) has been established to be commensurate with a diameter of the cutter/punch used to form the opening O, the cone 562/delivery assembly distal regions 672 can be moved relative to the exterior surface 812 of the second structure 810 and simply “slid” under the surgeon's finger and transferred into the opening O. To this end, the cone tip 572 (where provided) directly contacts and slides along the exterior surface 812; the smooth and deflectable characteristics of the cone tip surface 584 minimize or eliminate the possibilities of damaging the tissue of the exterior surface 812 as part of this sliding movement. Further, a diameter/size of the opening O can correspond with an effective diameter of the cone 562 such that upon insertion of the cone 562 within the opening O, hemostasis is attained, with blood flow out of the opening O being impeded/prevented by the cone 562. With this one acceptable approach, little or no liquid will escape from the second structure 810, and device insertion/hemostasis can quickly be accomplished. Placement of the cone 562 (and thus the delivery assembly distal regions 672 (hidden) and the tubular graft structure 800) within the opening O is shown in FIGS. 35B and 35C. As a point of reference, the contact surface 390 of the nose 372 bears against the exterior surface 812, thus dictating an extent of extension of the apparatus 620 within the opening O.

Once the cone 562/distal regions 672 have been inserted within the opening O, the transfer cap 560 can be removed from a remainder of the surgical connection apparatus 620 as shown in FIG. 35D. For example, the surgeon can grasp the handle 566 (either with a clamp-type tool or with the surgeon's fingers) and pull the handle 566 upwardly and transversely away from the opening O. Regardless, the transfer cap 560 is readily removed from a remainder of the apparatus 620 and the tubular graft structure 800.

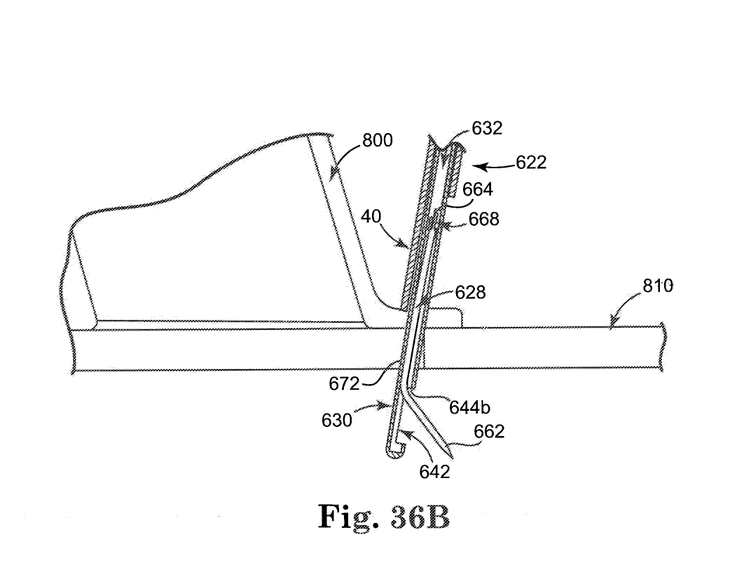

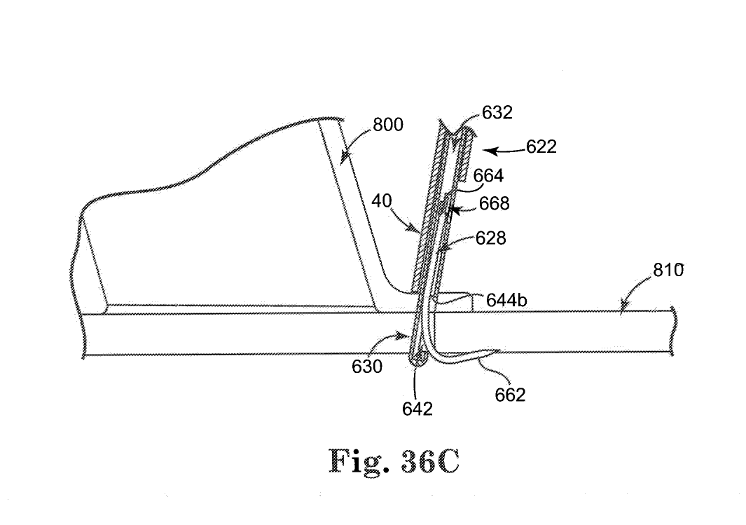

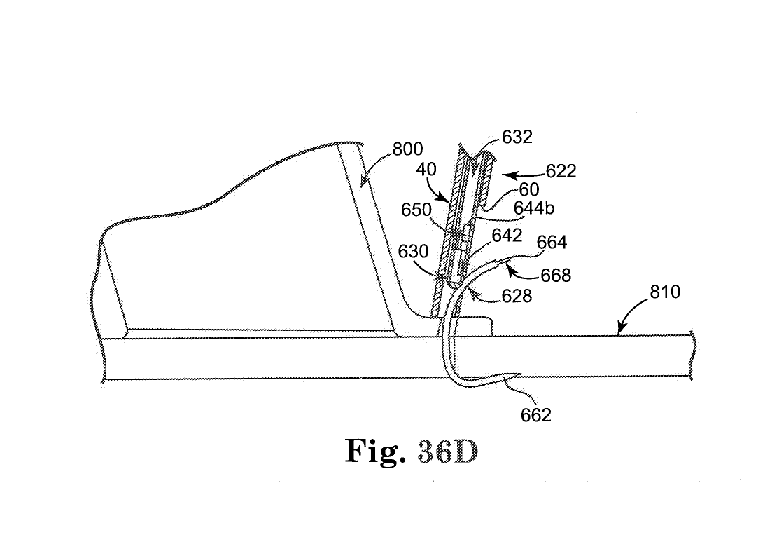

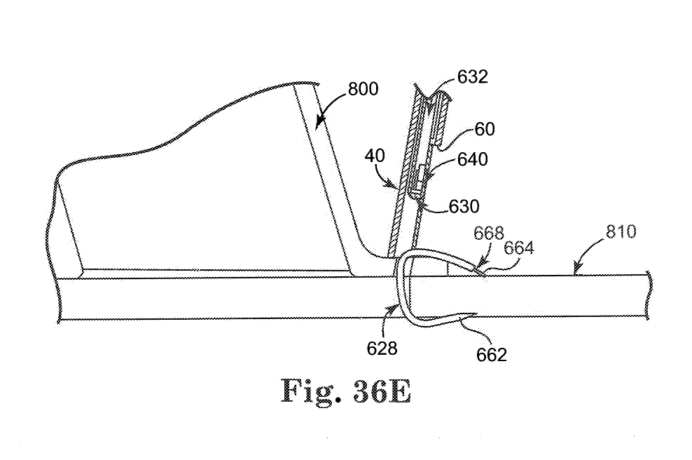

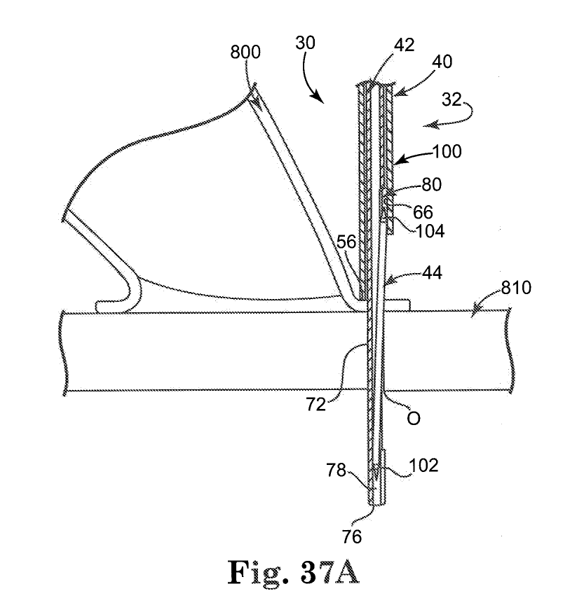

FIG. 36A illustrates an initial relationship of one of the delivery assemblies 622, including the corresponding clip 628 (otherwise “loaded” within the delivery assembly 622 in a pre-deployment state), relative to the tubular graft structure 800 and the second structure 810 upon initial placement of the distal region 672 within the opening O (and the transfer cap 560 (FIG. 35D) removed). As previously described, in the pre-deployment state of FIG. 36A, the clip 628 is captured within the distal region 672, with the first end 662 being proximal the window 642. The clip 628 does not contact the tubular graft structure 800 or the second structure 810. The tubular graft structure 800 is, however, mounted to the delivery assembly 622, for example along the inner member 630 and in approximate abutment with the distal end 56 of the outer member 40. Finally, the distal segment 636 of the inner member 630 projects into the opening O in the second structure 810.

The surgical connection apparatus 620 is then operated as described above to deploy the clip 628 into securement with the tubular graft structure 800 and the second structure 810 (e.g., with additional reference to FIG. 27B, the locking assembly 152 is manipulated to permit firing of the drive assembly 674 with depression of the actuator 254). In particular, and as shown in FIG. 36B, the inner member 630 is slid proximally relative to the outer member 40 and the retention member 632. In other words, the outer member 40 and the retention member 632 remain stationary whereas the inner member 630 moves proximally. Due to engagement between the retention member 632 and the clip 628, the second end 664/head 668 of the clip 628 remains spatially stationary with initial, proximal movement of the inner member 630. Proximal movement of the inner member 630 relative to the retention member 632/clip 628 continues until the first end 662 of the clip 628 is within the window 642 of the inner member 630 (i.e., proximal movement of the inner member 630 has progressed to a point at which the first end 662 is distal the proximal end 644b of the window 642). With the first end 662 of the clip 628 now free of the confines of the inner member 630, the shape memory attribute of the clip 628 causes the clip 628 to revert toward the undeflected or natural state such that the first end 662 (and any other portion of the clip 628 that is “exposed” at the window 642) is self-driven outwardly from the inner member 630. However, due to engagement between the head 668 and the retention member 632 (as well as capturing of the head 668 within the inner member 630), the clip 628 does not entirely disengage from the delivery assembly 622. Rather, only that portion of the clip 628 otherwise distal the window 642 self-reverts toward the undeflected or natural state.

With continued proximal movement of the inner member 630 relative to the retention member 632/clip 628, an increasing portion of the clip 628 is “exposed” and allowed to revert toward the undeflected state, with the first end 662 piercing into the second structure 810 as shown in FIG. 36C. Once again, the clip 628 remains partially captured by the delivery assembly 622 such that the state of FIG. 36C is achieved without entire release of the clip 628 from the delivery assembly 622.

With reference to FIG. 36D, proximal movement of the inner member 630 continues, with the window 642 eventually aligning with the slot 650 of the retention member 632 (and thus the head 668 of the clip 628). At this point, the head 668 is no longer circumferentially retained by the inner member 630. Thus, due to the shape memory characteristic of the clip 628, the clip 628 self-releases from the retention member 632, with the head 668 passing through the window 642. Further, due to the aligned relationship of the window 642 relative to the aperture 60 of the outer member 40, release of the clip 628 relative to the inner member 630 and the retention member 632 is not impeded by the outer member 40. Instead, the clip 628 freely passes through the aperture 60 of the outer member 40. As shown in FIG. 36E, the second end 664 pierces into the second structure 810, such that the clip 628 envelops (and/or pierces through) the distal end 808 of the tubular graft structure 800. In the deployed state of FIG. 36E, then, the clip 628 secures the tubular graft structure 800 to the second structure 810. The surgical connection apparatus 620 can then be removed from the surgical site.

As a point of reference, the surgical connection apparatus 620 is adapted such that transition of the delivery assemblies 622 from the pre-deployment state of FIG. 36A to the deployed state of FIG. 36E occurs relatively instantaneously. In other words, the drive mechanism 624 (FIG. 27A) is adapted such that once actuated, the inner member 630 is rapidly driven proximally relative to the corresponding outer member 40 and retention member 632. In this regard, the drive mechanism 624 can include the drive assembly 674/locking assembly 152 (FIG. 27A) arrangement as previously described, whereby in response to a single user actuation movement, the inner member 630 rapidly moves proximally to release the clip 628. To this end, the user actuation motion can be in a direction perpendicular to a direction of movement of the inner member 630. Regardless, the drive mechanism 624 is adapted such that the delivery assemblies 622 are simultaneously operated to effectuate substantially simultaneous deployment of the clips 628.

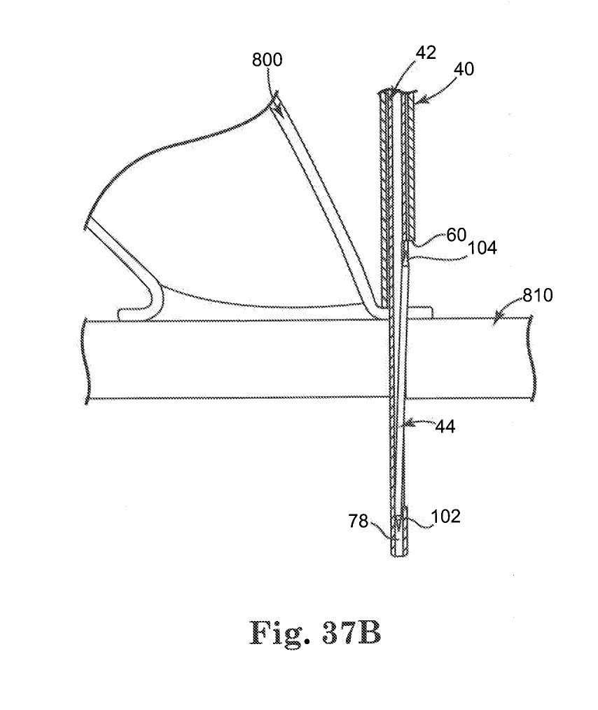

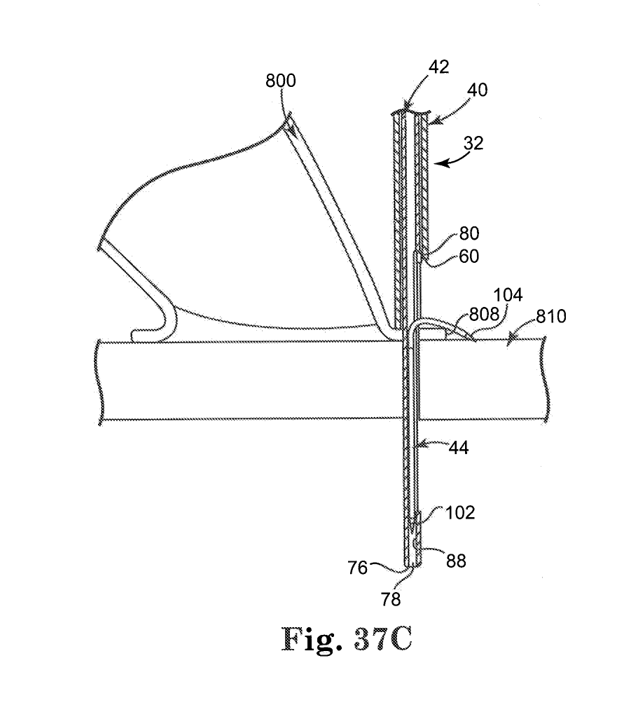

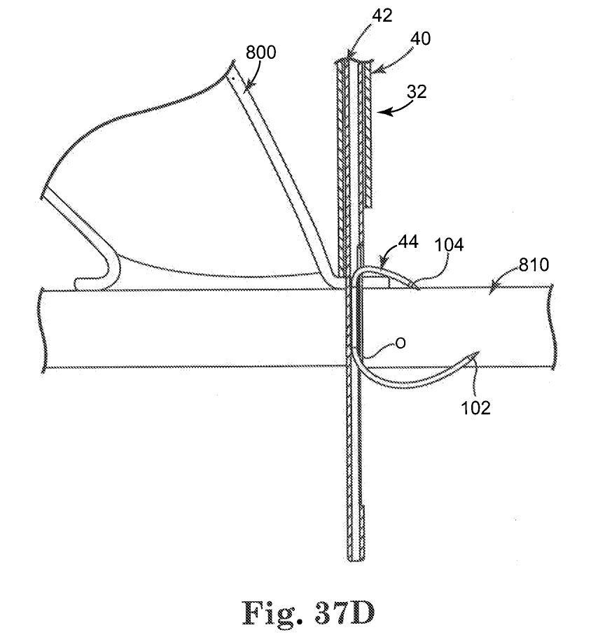

The surgical connection apparatus 30 (FIG. 1A) operates in a somewhat similar manner in deploying the clips 44 (FIG. 2). In general terms, FIGS. 37A-37D illustrate deployment of the clip 44 from one of the delivery assemblies 32. Following placement of the distal region 100 relative to the second structure 810 such that the inner member 42 is within the opening O (FIG. 37A), the inner member 42 is driven distally relative to the outer member 40. The clip 44 is carried by the inner member 42, with the second end 104 sliding along the outer member 40. Distal movement of the inner member 42/clip 44 continues to the position of FIG. 37B, in which the second end 104 is aligned with, and thus releasable through, the aperture 60 of the outer member 40. Once released, the clip 44 self-reverts toward the natural or undeflected state, such that the second end 104 pierces into the second structure 810 as shown in FIG. 37C. With further distal movement of the inner member 42, an interface between the second end 104 and the second structure 810 (as well as between the clip 44 and the graft structure 800) overcomes a capture force established between the inner member 42 and the first end 102. Thus, the clip 44 does not move distally with further distal movement of the inner member 42 from the positions of FIG. 37C. Instead, the first end 102 slides along the inner member 42 until reaching the window 80. At this point, first end 102 is released from the passage 78 of the inner member 42 (via the window 80), with the clip 44 self-reverting toward the undeflected state as reflected in FIG. 37D. The first end 102 pierces through at least the second structure 810 such that the clip 44 secures the tubular graft structure 800 to the second structure 810. The drive mechanism 34 optionally then proximally retracts the inner member 42 as previously described.

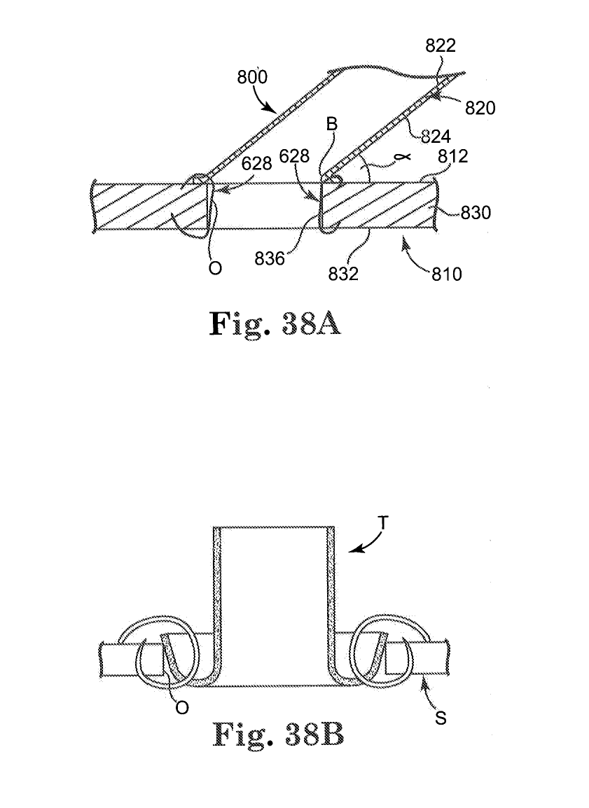

Anastomosis between the tubular graft structure 800 and the second structure 810 achieved with the surgical connection apparatuses 30 (FIG. 1A), 620 (FIG. 27A) in accordance with some aspects of the present disclosure is shown in FIG. 38A. In particular, a plurality of the clips 628 secure the tubular graft structure 800 to the second structure 810 in a region of the opening O. Due to the bevel cut described above, the tubular graft structure 800 extends at a non-right angle fashion relative to the exterior surface 812 of the second structure 810. That is to say, upon completion of the anastomosis procedure, the tubular graft structure 800 extends at an angle α relative to the top surface 812 that is less than 90° (e.g., in the range of 30°-70°). This is distinguishable from the right angle take-off that is normally effectuated by use of conventional, automated anastomosis tools (and reflected in FIG. 38B), and represents a marked improvement. For example, where the take-off angle α is properly selected by the surgeon, the tubular graft structure 800 will not be subsequently forced through various bends/angles in directing a proximal portion (not shown) thereof to a desired anatomical location. This, in turn, means that the tubular graft structure 800/second structure 810 interface will not unnecessarily be subjected to external forces that might otherwise negatively impact long-term securement.

An additional unique characteristic provided by aspects of the present disclosure relates to the surface(s) of interface between the tubular graft structure 800 and the second structure 810 resulting from use of the apparatuses and methods described. In this regard, the tubular graft structure 800 includes a wall 820 defining an inner surface 822 and an outer surface 824. Similarly, the second structure 810 includes a wall 830 defining an interior surface 832 and the exterior surface 812. Further, a perimeter of the opening O is defined by an opening surface 836 extending between the interior and exterior surfaces 832, 812. With these conventions in mind, the surgical connection apparatuses 30 (FIG. 1A) or 620 (FIG. 27A) and corresponding methods of use are adapted such that following anastomosis, the inner surface 822 of the tubular graft structure 800 abuts the exterior surface 812 of the second structure 810, with the clips 628 establishing a sealed relationship therebetween. In some embodiments, the anastomosed relationship includes the tubular graft structure 800 not extending, or minimally extending, along the opening surface 836. That is to say, a thickness of the tubular graft structure 800 does not overtly reduce an effective size of the opening O. In other embodiments, the anastomosed relationship includes the tubular graft structure 800 not extending into contact with the interior surface 832 of the second structure 810. Stated otherwise, the distal portion 802 of the tubular graft structure 800 forms only a single bend B at the corresponding point of interface with the clips 628. Thus, the anastomosed relationship provided with the surgical connection apparatuses 30, 620 and related methods of use in accordance with aspects of the present disclosure are akin to a hand-sewn technique, but can be achieved in a fraction of the time required for manual sewing and includes interrupted points of attachment. Finally, the circumferentially spaced arrangement of the clips 628 renders the tubular graft structure 800/second structure 810 interface highly compliant, able to grow and contract with normal variations in blood flow/volume through the interface.

As a point of reference, FIG. 38B illustrates anastomosis of a tubular graft structure T to a second structure S using a previous clip deployment apparatus. As shown, the tubular graft structure T extends within the opening O of the second structure S, thus reducing an effective size of the opening O. More particularly, the tubular graft structure T extends within and is folded relative to the opening O, such that a thickness of the tubular graft structure T reduces an effective size of the opening O. Further, a right angle take-off is established by the tubular graft structure T relative to the second structure S.

### Kit

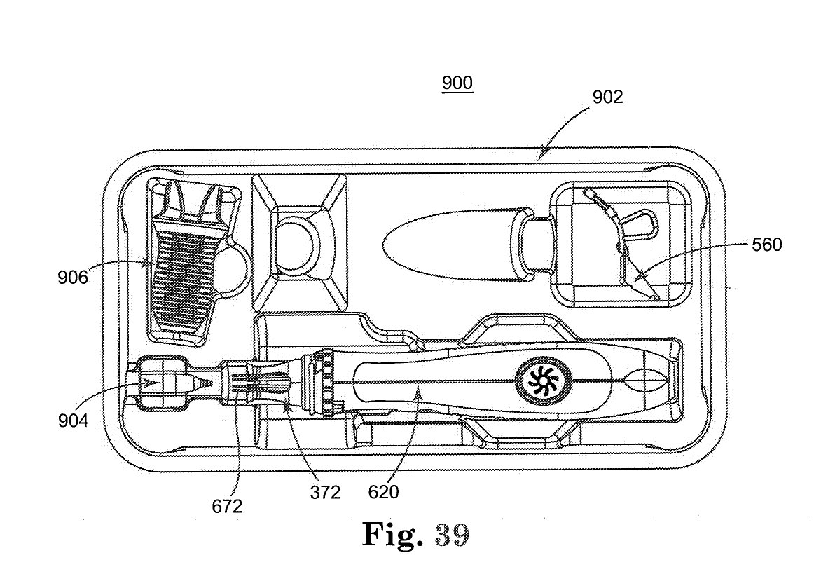

In addition to providing an improved surgical connection apparatus that operates to perform a right angle takeoff and anastomosis procedure in a simple and ergonomically conducive manner, other aspects in accordance with the principles of the present disclosure relate to a kit adapted to facilitate the procedure, and in particular mounting of the tubular graft structure 800 onto the surgical connection apparatus 30 or 620. For example, FIG. 39 illustrates one embodiment of a kit 900 including the surgical connection apparatus 620 and the transfer cap 560 as previously described, along with a tray 902, a loading pin 904, and a guide piece 906. In general terms, the tray 902 is configured to selectively receive the surgical connection apparatus 620, the loading pin 904, and the guide piece 906, as well as to promote desired interface between the components 620, 904 in loading a tubular graft structure T (FIG. 38A) to the surgical connection apparatus 620. In addition, the kit 900 can include other components, such as a sizer (not shown) and/or one or more cutting devices (not shown) such as an aortotomy punch.

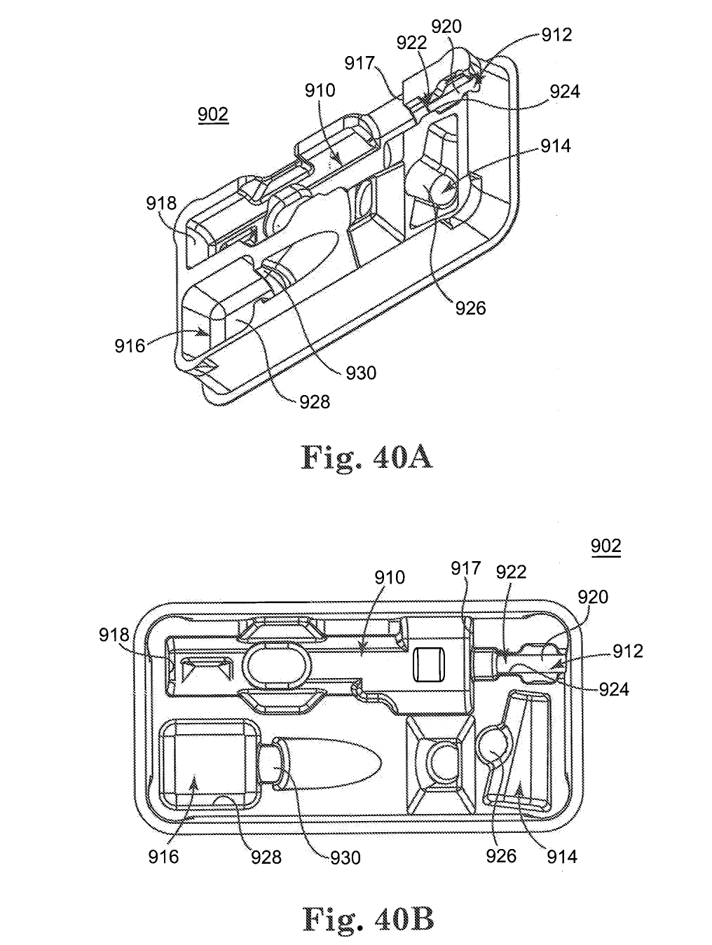

The tray 902 can assume a variety of forms and sizes. With respect to the but one acceptable configuration shown in FIGS. 40A and 40B, the tray 902 generally forms or defines a tool pocket 910, a loading station 912, a guide piece pocket 914, and a temporary storage station 916. The tool pocket 910 is sized and shaped to slidably retain the surgical connection apparatus 620 (FIG. 39) such that the surgical connection apparatus 620 can slide axially relative to the loading station 912. The tool pocket 910 is generally defined by a leading side 916 and a trailing side 918, with the leading side 916 being adjacent the loading station 912.

The loading station 912 is adapted to selectively retain the loading pin 904 (FIG. 39), for example via a well 920. Further, a channel 922 is formed between the well 920 and the leading side 916 of the tool pocket 910. The channel 922 is sized to receive a portion of the surgical connection apparatus 620 (FIG. 39) as described below, and can include a reduced width portion 924.

The guide piece pocket 914 is sized to selectively (e.g., frictionally) retain the guide piece 906 (FIG. 39). In this regard, the guide piece pocket 914 can include or form a groove 926 through which a user's finger can be inserted when removing the guide piece 906 from the pocket 914.

The temporary storage station 916 is sized for storing the transfer cap 560 (FIG. 39). In addition, the temporary storage station 916 can be utilized in temporarily holding a tubular graft structure, otherwise loaded to the surgical connection apparatus 620 (FIG. 39), prior to use. For example, the temporary storage station 916 can include or define a cup 928 and a slot 930. A depth of the cup 928 is greater than that of the slot 930. Further, a width of the slot 930 is slightly less than a corresponding dimension of the surgical connection apparatus 620 (e.g., a dimension of the nose 372). With this configuration, then, the cup 928 can be at least partially filled with an appropriate liquid (e.g., saline) and the surgical connection apparatus 620 temporarily held at the slot 930, with the distal regions 672 (and thus a tubular graft structure mounted thereto) extending into the cup 928. As a result, the surgical connection apparatus 620 is retained in this orientation via the slot 930, with the tubular graft structure being immersed within the liquid of the cup 928 prior to use.

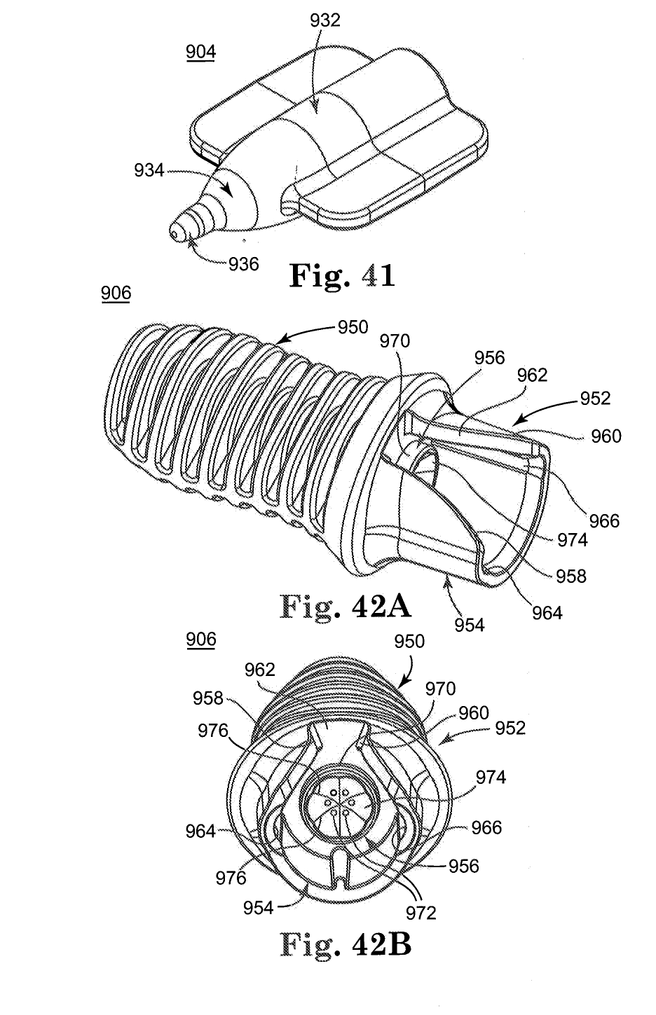

The loading pin 904 is shown in FIG. 41, and includes a base 932, a tapered segment 934, and a shaft 936. The base 932 and the well 920 (FIG. 40A) are correspondingly sized such that the base 932 can be frictionally retained within the well 920. The tapered segment 934, in turn, extends from the base 932, tapering in outer diameter. As described below, the tapered segment 934 provides a surface along which a tubular graft structure can be distended. Finally, the shaft 936 extends from the tapered segment 934, and facilitates centering of the tubular graft structure relative to the tapered segment 934 during use.

The guide piece 906 is shown in greater detail in FIGS. 42A and 42B, and generally includes a grip 950 and a guide portion 952. The grip 950 is sized and shaped for convenient grasping and handling by a user's hand. The guide portion 952 extends from the grip 950 and includes a flange 954 and a centering body 956. The flange 954 projects from the grip 950, and surrounds the centering body 956. Further, the flange 954 includes first and second edges 958, 960 that are separated from one another to define a gap 962 (referenced generally). As described below, the gap 962 is sized to permit passage of a tubular graft structure (not shown) therethrough. In addition, opposing ruts 964, 966 are formed along an interior of the flange 954, extending in a generally longitudinal fashion. As described below, the ruts 964, 966 are adapted to slidably mate with corresponding features of the surgical connection apparatus 620 (FIG. 39) in effectuating a desired orientation of the surgical connection apparatus 620 relative to the centering body 956.

The centering body 956 is generally cylindrical, defining a circular perimeter 970. With specific reference to FIG. 42B, a plurality of equidistantly spaced holes 972 are circumferentially arranged within the perimeter 970. The number of holes 972 corresponds with the number of delivery assemblies 622 (FIG. 27A) provided with the surgical connection apparatus 620 (FIG. 39). Further, an effective diameter selectively defined by the holes 972 generally corresponds with, or is slightly less than, an effective diameter collectively defined by the distal regions 672 (FIG. 27B) in the radially contracted state as described above. A surface 974 of the centering body 956 tapers inwardly (i.e., toward the grip 950) in extension from the perimeter 970 to the holes 972. In addition, a plurality of ribs 976 are formed on the surface 974, with individual ones of the ribs 976 being radially positioned between an adjacent pair of the holes 972.

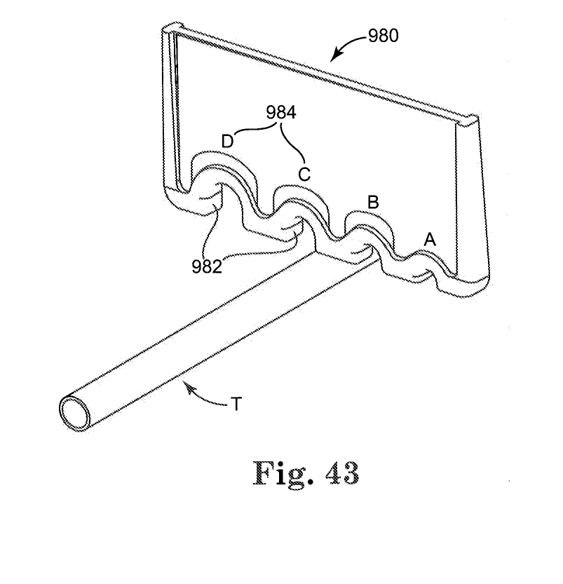

With the above components in mind and returning to FIG. 39, the kit 900 expedites loading of a tubular graft structure onto the surgical connection apparatus 620. For example, a size of the tubular graft structure to be anastomosed is initially evaluated. A sizing device 980 of FIG. 43 (that can otherwise be provided with the kit 900) can assist in this evaluation. The sizing device 980 includes, in some embodiments, a series of differently sized apertures 982 and corresponding indicia 984. The indicia 984 correlates the evaluated size of the corresponding aperture 982 with a desired surgical connection apparatus 620 (e.g., number of delivery assemblies 622). Based upon this size evaluation, an appropriately configured surgical connection apparatus 620 can be selected (e.g., depending upon a size of the tubular graft structure T, the surgical connection apparatus 620 can be selected having an appropriate number of the delivery assemblies 622). In some embodiments, a plurality of different surgical connection apparatuses 620 will be available to the surgeon, each labeled with a unique identifier corresponding with one of the sizer indicia 984. Upon determining which of the apertures 982 the tubular graft structure T best matches, the corresponding indicia 984 is compared with the apparatus labels, and the so-identified surgical connection apparatus is then selected by the surgeon.

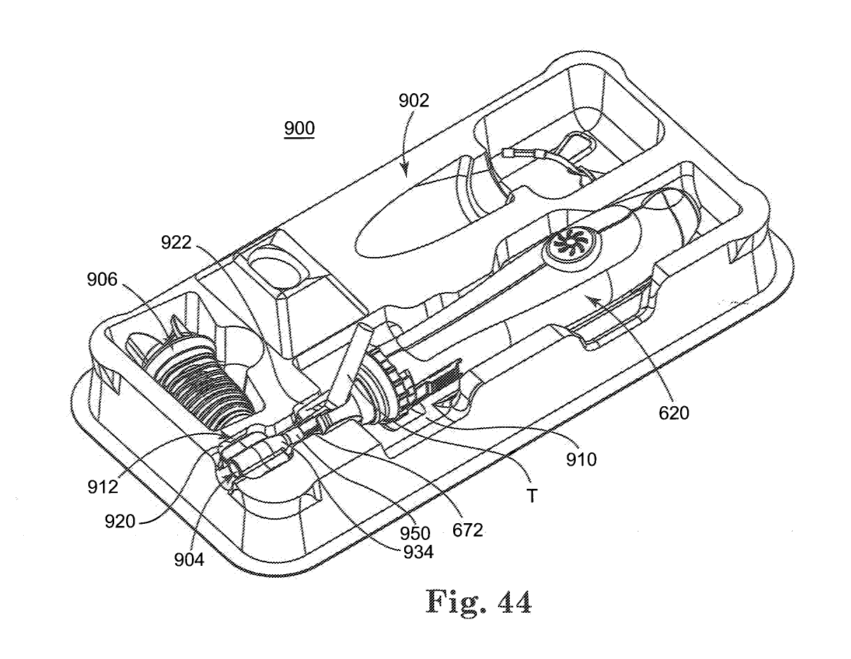

The tubular graft structure T is then placed over the loading pin 904 such that the tapered segment 934 distends a distal portion 950 thereof as shown in FIG. 44. The loading pin 904/tubular graft structure T is then placed in the loading station 912. In particular, the loading pin 904 is inserted into the well 920 such that the tubular graft structure T is within the channel 922 and “faces” the tool pocket 910. The surgical connection apparatus 620 is similarly loaded in the tool pocket 910 such that the exposed delivery assembly distal regions 672 are within the channel 922 and “face” the loading station 912.

The surgical connection apparatus 620 is then slid within the pocket 910 toward the loading pin 904/tubular graft structure T. The delivery assembly distal regions 672 contact and may partially pierce through the tubular graft structure T.

Once the delivery assemblies distal regions 672 are in contact with the tubular graft structure T, the user (not shown) can then move the tubular graft structure T proximally along the delivery assembly distal regions 672 (for example, employing a tweezers or similar tool). The apparatus 620/graft structure T can remain in the tray 902 or removed therefrom during this process, and the loading pin 904 can be in contact with, or removed from, the graft structure T. Once at least partially loaded with the tubular graft structure T, the surgical connection apparatus 620 is removed from the tray 902, along with the guide piece 906. Further, the loading pin 904 is withdrawn from the graft structure T.

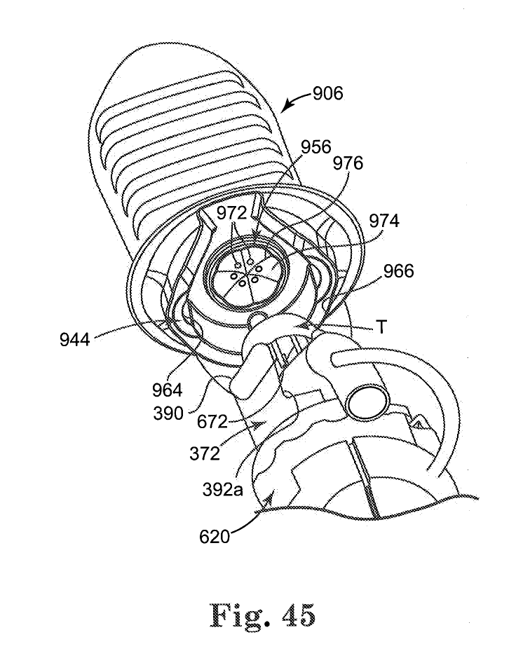

As shown in FIG. 45, the guide piece 906 can then be employed to proximally move the tubular graft structure T along the distal regions 672. In particular, the distal regions 672 are generally aligned with the centering body 956. The flange 944 serves to dictate a desired orientation of the surgical connection apparatus 620 relative to the centering body 956 via mating interface between the ruts 964, 966 and the nose 372. As the distal regions 672 are slid toward the holes 972, the gap 962 permits passage of the tubular graft structure T. Further, the tapered surface 974 of the centering body 956 along with the ribs 976 guide or direct individual ones of the distal regions 672 into corresponding ones of the holes 972.

With further movement of the surgical connection apparatus 620 relative to the guide piece 906 (and/or vice-versa), the centering body 956 abuts against the tubular graft structure T, thus forcing or sliding the tubular graft structure T proximally along the delivery assembly distal regions 672. Forced proximal movement of the tubular graft structure T continues until the tubular graft structure T contacts or abuts the contact surface 390 (referenced generally) of the nose 372. At this point of location, the tubular graft structure T is loaded to the delivery assembly distal regions 672 at a desired position. This desired location is further facilitated by the first side opening 392a of the nose 372; the tubular graft structure T can be extended through the opening 392a as shown to promote ease of handling. The surgical connection apparatus 620 can then be retracted relative to the guide piece 906 and is then prepared for performance of the anastomosis procedure as described above that may or may not include use of the transfer cap 560 (FIG. 39).

It will be understood that the kit 900 can assume a variety of forms differing from the above description. Even further, the surgical connection apparatuses of the present disclosure are highly useful apart from the other components of the kit 900.

The surgical connection apparatuses and related methods of use present marked improvements over previous designs. A plurality of self-closing clips are simultaneously delivered in performing an anastomosis procedure, with the resulting anastomosed tubular graft structure forming a non-right angle takeoff relative to the structure to which it is secured if desired. In other embodiments, the surgical connection apparatus is provided in conjunction with a kit providing additional components that facilitate expedited loading of the tubular graft structure to the surgical connection apparatus in a consistent manner.

Although the present disclosure has been described with reference to preferred embodiments, workers skilled in the art will recognize that changes can be made in form and detail without departing from the spirit and scope of the present disclosure.

What is claimed is:

1. A surgical connection apparatus comprising:
a plurality of delivery assemblies each including:
an outer member defining a proximal end, a distal end, and a lumen,
an inner member slidably disposed within the lumen and defining a distal section terminating at a distal tip, a proximal section, an internal passage, and a longitudinal window open to the internal passage, the window being formed in the distal section and terminating proximal the distal tip,
wherein the delivery assemblies each define a distal region and are adapted to releasably retain a self-closing clip within the respective internal passage adjacent the window in a retained state;
a housing assembly maintaining the plurality of delivery assemblies such that the distal regions are generally circularly arranged; and
a drive mechanism for transitioning the delivery assemblies from the retained state to effectuate release of the self-closing clips from the delivery assemblies.

2. The apparatus of claim 1, wherein the delivery assemblies are each configured to provide the retained state in which the self-closing clip is captured within the delivery assembly and a released state in which the self-closing clip can be released from the delivery assembly, and further wherein the retained state includes the inner member distal tip extending distal the distal end of the corresponding outer member.

3. The apparatus of claim 2, wherein the delivery assemblies are each configured to transition from the retained state to the released state by sliding the inner member relative to the outer member.

4. The apparatus of claim 3, wherein the inner members of each of the delivery assemblies are configured to retain an end of the self-closing clip when sliding relative to the outer member from the retained state.

5. The apparatus of claim 1, wherein each of the delivery assemblies further includes a retention member slidably received within the corresponding inner member, a distal section of the retention member configured to selectively engage the corresponding clip.

6. The apparatus of claim 5, wherein the delivery assemblies are each configured to provide the retained state in which the self-closing clip is captured within the delivery assembly and a released state in which the clip can be released from the delivery assembly, and further wherein the apparatus is configured to transition from the retained state to the released state by the drive mechanism moving the inner members proximally relative to the corresponding retention members.

7. The apparatus of claim 6, wherein the apparatus is configured such that for each of the delivery assemblies in the retained state, a second end of the clip is connected to the distal section of the retention member and a first end of the clip is distal the second end and bears against an inner surface of the inner member, proximal the window.

8. The apparatus of claim 6, wherein the inner member of each of the delivery assemblies is configured to define a retention surface for slidably capturing the second end of the corresponding self-closing clip.

9. The apparatus of claim 1, wherein relative to the circular arrangement of the delivery assembly distal regions, the window of each of the outer members faces radially outwardly.

10. The apparatus of claim 1, wherein each of the outer members is a tubular sheath.

11. The apparatus of claim 1, wherein each of the inner members is an elongated tube.

12. The apparatus of claim 1, wherein the housing assembly includes a housing and a nose extending distal the housing, and further wherein the nose exteriorly surrounds the plurality of delivery assemblies and forms at least one side opening configured for passage of a tubular graft structure.

13. The apparatus of claim 1, further comprising:
a retention clasp pivotably maintained by the housing assembly adjacent the distal regions and configured to selectively retain a tubular graft structure against a housing of the housing assembly.

14. The apparatus of claim 1, wherein the drive mechanism is configured to simultaneously slide the inner members relative to the corresponding outer members.

15. The apparatus of claim 1, wherein each of the delivery assemblies further includes a retention member slidably disposed within the corresponding inner member, and further wherein the drive mechanism includes:
a first holder body attached to each of the outer members;
a second holder body attached to each of the inner members;
a third holder body attached to each of the retention members; and
a biasing assembly for biasing the second holder body relative to the third holder body.

16. The apparatus of claim 15, wherein the housing assembly maintains the drive mechanism such that the second holder body is axially slidable relative to the third holder body.

17. The apparatus of claim 15, wherein the delivery assemblies are each configured such that the proximal section of the inner member extends proximally from the proximal end of the corresponding outer member, and further wherein the second holder body is maintained by the housing assembly proximal the first holder body.

18. The apparatus of claim 15, wherein the drive mechanism further includes a locking assembly for releasably locking the second holder body relative to the third holder body in a locked state, the locking assembly including an actuator accessible from an exterior of the housing assembly for transitioning the locking assembly from the locked state, and further wherein the apparatus is configured such that a direction of movement of the actuator is generally perpendicular to a direction of movement of the inner members relative to the outer members.

19. The apparatus of claim 1, further comprising:
an adjustment device connected to the plurality of delivery assemblies for adjustably dictating a radial spacing of the distal regions.

20. The apparatus of claim 19, wherein the adjustment device includes a ring accessible from an exterior of a housing of the housing assembly, and further wherein the adjustment device is configured to effectuate a change in the radial spacing of the distal regions in response to rotation of the ring relative to the housing.

21. The apparatus of claim 20, wherein the adjustment device further includes:
a first disc forming a plurality of drive slots each sized to slidably receive a respective one of the outer members, the first disc being assembled to the ring;
wherein upon final assembly, rotation of the first disc causes the distal ends of the outer members to deflect radially inwardly or outwardly relative to one another as the outer member slides within the corresponding drive slot.

22. The apparatus of claim 21, wherein the adjustment device further includes:
a second disc forming a plurality of guide slots each sized to receive a corresponding one of the outer members;
wherein upon final assembly, the second disc remains stationary with rotation of the first disc relative to the housing.

23. A method of performing an anastomosis comprising:
providing a surgical connection apparatus including a plurality of delivery assemblies each defining a distal region and releasably retaining a self-closing clip;
passing the distal regions through a wall of a tubular graft structure adjacent a distal end thereof,
introducing the delivery assembly distal regions into an opening formed in a second structure, the second structure defining an exterior surface opposite an interior surface; and
simultaneously deploying a plurality of clips from the delivery assemblies such that the clips pass through the tubular graft structure and the second structure to secure the tubular graft structure and the second structure together;
wherein upon securement, an interface region is defined in the tubular graft structure from a location at which the clips pass through the tubular graft structure and the distal end thereof, and further wherein at least a majority of the interface region abuts the exterior surface of the second structure.

24. The method of claim 23, wherein securement of the tubular graft structure and the second structure includes the interface region forming a single bend.

25. The method of claim 23, wherein securement of the tubular graft structure and the second structure includes the location at which the clips pass through the tubular graft structure being closer to the exterior surface of the second structure than the interior surface.

26. The method of claim 23, wherein securement of the tubular graft structure to the second structure includes the interface region not extending beyond the interior surface opposite the exterior surface.

27. The method of claim 23, wherein securement of the tubular graft structure and the second structure includes the interface region not extending into the opening.

28. The method of claim 23, wherein introducing the distal regions into the opening of the second structure includes each of the clips remaining entirely within the corresponding delivery assembly.

29. The method of claim 28, wherein each of the clips includes a first clip end and a second clip end, and further wherein prior to deployment, the first clip end is positioned distal the second clip end, and further wherein deploying the clips includes:
a) deploying the first clip end from the corresponding delivery assembly such that the first clip end initially pierces through the interior surface of the second structure; and
b) deploying the second clip end of from the corresponding delivery assembly such that the second clip end is adjacent the exterior surface of the second structure.

30. The method of claim 29, wherein step b) includes the second clip end piercing through the tubular graft structure.

31. The method of claim 23, wherein each of the delivery assemblies includes an retention member slidably retained within an inner member, the inner member slidably retained within an outer member and forming a passage for retaining a clip and a window through which the clip is releasable from the passage, and further wherein deploying the clips includes:
simultaneously sliding the inner members proximally relative to the corresponding retention members.

32. The method of claim 31, wherein each clip includes a first clip end and a second clip end and is arranged within the corresponding delivery assembly such that the first clip end is distal the second clip end, and further wherein with proximal sliding of the inner members, the first clip end is first released from the inner member followed by release of the second clip end from the inner member.

33. The method of claim 32, wherein the surgical connection apparatus includes a drive mechanism for causing the inner members to slide relative to the corresponding retention members, and further wherein simultaneously deploying the clips includes:
a user pressing a single actuator button to cause movement of the drive mechanism.

34. The method of claim 23, wherein passing the distal regions through the wall of the tubular graft structure includes:
distending the distal end of the tubular graft structure to a distended diameter;
arranging the distal regions to a generally circular arrangement having a diameter less than the distended diameter; and
projecting the distal regions through the tubular graft structure while the distal end is distended.

35. The method of claim 34, wherein the surgical connection apparatus includes a housing and an adjustment device including a ring connected to the plurality of delivery assemblies, and further wherein arranging the distal regions includes rotating the ring relative to the housing.

36. The method of claim 23, wherein the method is characterized by the absence of barbs passing through the tubular graft structure prior to deploying the clips.

37. The method of claim 23, further comprising:
cutting the tubular graft structure to form a bevel cut distal end prior to passing the distal regions of the surgical connection apparatus through the tubular graft structure;
wherein securement of the tubular graft structure and the second structure includes the tubular graft surface extending from the exterior surface of the second structure at an extension angle that is less than 90°.

38. The method of claim 37, wherein the extension angle is in the range of 30°-70°.

39. The method of claim 23, further comprising:
assembling a transfer cone about a portion of the tubular graft structure and the distal regions prior to introducing the distal regions into the opening;
wherein introducing the distal regions into the opening includes inserting the transfer cone into the opening to attain hemostasis at an interface between the cone and the opening.

40. A kit for performing anastomosis comprising:
a surgical connection apparatus comprising:
a plurality of delivery assemblies each including:
an outer member defining a proximal end, a distal end, and a lumen,
an inner member slidably disposed within the lumen and defining a distal section terminating at a distal tip, an internal passage, and a longitudinal window open to the internal passage,
wherein the delivery assemblies each define a distal region and are adapted to releasably retain a self-closing clip,
a housing assembly maintaining the plurality of delivery assemblies such that the distal regions are generally circularly arranged,
a drive mechanism for transitioning the delivery assemblies from a retained state to a released state to effectuate release of the self-closing clips from the delivery assemblies; and
a loading tray including:
a loading station for maintaining a tubular graft structure,
a tool pocket sized to slidably receive the surgical connection apparatus;
wherein the tool pocket is arranged relative to the loading station such that upon positioning of a tubular graft structure in the loading station, the surgical connection apparatus is slidable along the pocket to effectuate loading of the tubular graft structure onto the delivery assemblies.

41. The kit of claim 40, further comprising:
a loading pin defining a tapered surface and adapted to be selectively secured within the loading station.

42. The kit of claim 40, further comprising:
a guide piece forming a plurality of holes configured to slidably receive the delivery assemblies.

43. The kit of claim 40, further comprising:
a transfer cap including a cone configured for selective placement about the distal regions of the delivery assemblies.

44. The kit of claim 43, wherein the cone terminates in a tip and further wherein upon assembly to the surgical connection apparatus, the tip extends distal the distal regions.
